(19) United States
(12) Patent Application Publication
(10) Pub. No.: US 2007/0265231 A1
(43) Pub. Date: Nov. 15, 2007

(54) Hydrazide Compounds for Combating Animal Pests

(76) Inventors: Michael Hofmann, Ludwigshafen (DE); Michael Rack, Heidelberg (DE); David G. Kuhn, Apex, NC (US); Deborah L. Culbertson, Fuquay Varina, NC (US); Douglas D. Anspaugh, Apex, NC (US)

Correspondence Address: HUTCHISON LAW GROUP PLLC, PO BOX 31686, RALEIGH, NC, 27612, US

(21) Appl. No.: 11/791,919

(22) PCT Filed: Nov. 30, 2005
(86) PCT No.: PCT/EP05/12787

§ 371 Date: May 30, 2007

(51) Int. Cl. A01N 33/26; A01P 5/00; A01P 7/00; C07C 243/18; C07C 243/42

(52) U.S. Cl. 514/150; 549/475; 552/6

(57) ABSTRACT

The present invention relates to new hydrazide compounds which are useful for combating animal pests, in particular insects, arachnids and nematodes and to the salts thereof. The invention also relates to a method for combating insects, nematodes and arachnids. The hydrazide compounds of the invention are described by the general formula (I) wherein . . . is absent or a covalent bond; A is an optionally substituted cyclic radical selected from phenyl, naphthyl and a 5- or 6-membered heterocyclic radical with 1 to 4 heteroatoms which are selected, independently of one another, from O, N and S, the 5- or 6-membered heterocyclic radical may have a carbonyl group as ring member; Q is selected from the group consisting of a single bond, C₁-C₄ alkylidene, O—-C₁-C₄ alkylidene, S—C₁-C₄ alkylidene and NR⁹—C₁-C₄ alkylidene, wherein the alkylidene group in the last four mentioned radicals is unsubstituted or carries 1, 2, 3 or 4 substituents selected from OH, ═O, halogen, C₁-C₄ haloalkyl and C₁-C₄ alkoxy; or A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy, X is C═O, C═S or SO₂; Ar is an optionally substituted aromatic radical selected from phenyl, naphthyl, pyridyl, pyrimidyl, furyl and thienyl; and R¹ to R⁶ and R⁹ are as described in the claims and the specification.

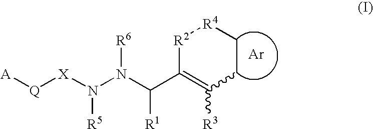

The present invention relates to new hydrazide compounds which are useful for combating animal pests, in particular insects, arachnids and nematodes. The invention also relates to a method for combating insects, nematodes and arachnids.

In spite of commercial pesticides available today, damage to crops, both growing and harvested, the damage of non-living material, in particular cellulose based materials such as wood or paper, and other nuisance, such as transmission of diseases, caused by animal pests still occur.

JP 2000169461 describes inter alia thiadiazolylcarbonylhydrazones of phenylketones having insecticidal or fungicidal activity. However, the insecticidal activity of these compounds is not satisfactory.

A. M. Islam et al., Egyptian Journal of Chemistry 1986, 29(4) S. 405-431 (CASREACT 111:173716) discloses several naphthalin-2-yl sulfonylhydrazones of aromatic aldehydes, which were screened against cotton leaf worm (Spodoptera literalis). However, the activity of these compounds against other pests is not satisfactory.

Therefore, there is continuing need to provide compounds which are useful for combating insects, nematodes and arachnids.

T. M. Temerk et al. Bull. Soc. Chim. Belg. 89(7),1980, S. 489-498 describe the polarographic reduction in accordance with the following scheme:

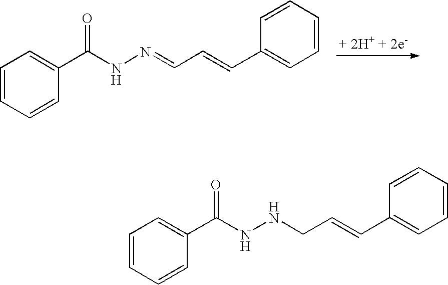

International application PCT/EP 2004/005681 discloses compounds of the general formula

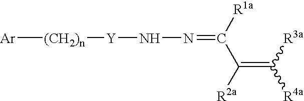

wherein Ar is an optionally substituted cyclic radical, selected from phenyl, napthyl and heterocyclic radicals, n is 0 or 1, Y is inter alia CO or SO₂, R^{1a} is H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl or optionally substituted phenyl, R^{2a} and R^{3a} are inter alia H, C₁-C₁₀-alkyl, C₁-C₁₀-haloalkyl, halogen, optionally substituted phenyl or cyano and R^{4a} is inter alia an optionally substituted aromatic radical selected from phenyl, pyridyl, pyrimidyl, furyl and thienyl. These compounds are active against insects and arachnids.

It is an object of the present invention to provide further compounds having a good activity against insects, nematodes and/or arachnids and thus are useful for combating said pests.

The inventors of the present application surprisingly found that this object is achieved by compounds of formula I as defined below and the salts thereof. These compounds have not yet been described, except for a compound of formula I, wherein A and Ar are unsubstituted phenyl, Q is a single bond, X is C═O and R¹, R², R³, R⁵ and R⁶ are hydrogen.

Therefore, the present invention relates to compounds of the general formula I

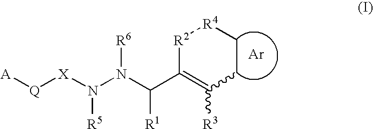

and to the salts thereof, wherein
..... is absent or a covalent bond;
A is a cyclic radical selected from phenyl and a 5- or 6-membered heterocyclic radical with 1 to 4 heteroatoms as ring members which are selected, independently of one another, from O, N and S, the 5- or 6-membered heterocyclic radical may have a carbonyl group as ring member, and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄ alkylidene, S—C₁-C₄ alkylidene and NR⁹—C₁-C₄ alkylidene, wherein the alkylidene group in the last four mentioned radicals is unsubstituted or carries 1, 2, 3 or 4 substituents selected from OH, ═O, halogen, C₁-C₄ haloalkyl and C₁-C₄ alkoxy;
   or the moiety A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above and wherein two radicals R^{c} which are bound to adjacent carbon atoms may form a O—CH₂—O moiety, where in said moiety 1 or 2 hydrogen atoms may be replaced by a radical R^{b} as defined above;
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, phenyl and a 5- or 6-membered heterocyclic radical with 1 to 4 heteroatoms which are selected, independently of one another, from O, N and S, it being possible for the aforementioned two last radicals to be unsubstituted or substituted by 1, 2, 3, 4 or 5 substituents R^{a} as defined above;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
   R⁴ together with R² is a bivalent radical Y, which is selected from O, S, CR¹⁰═N, N═N, O—CR¹¹R¹², S—CR¹¹R¹², N(R¹³)—CR¹¹R¹², O—C(O), O—C(S), S—C(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl optionally substituted by CN or NO₂, C₁-C₁₀-haloalkyl, C₃-C₁₀-cycloalkyl, C₃-C₁₀-halocycloalkyl, C₂-C₁₀alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkylcarbonyl, C₂-C₁₀-alkenylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, R⁷R⁸N—CO—, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b}; and
R¹⁰ is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};

except for a compound of formula I, wherein A and Ar are unsubstituted phenyl, Q is a single bond, X is C═O and R¹, R², R³, R⁵ and R⁶ are hydrogen.

Due to their excellent activity, the compounds of the general formula I can be used for controlling pests, selected from harmful insects, arachnids and nematodes. The compounds of the formula I are in particular useful from combating insects.

Accordingly, the invention further provides compositions for combating such pests, preferably in the form of directly sprayable solutions, emulsions, pastes, oil dispersions, powders, materials for scattering, dusts or in the form of granules, which comprises a pesticidally effective amount of at least one compound of the general formula I or at least a salt thereof and at least one carrier which may be liquid and/or solid and which is preferably agronomically acceptable, and/or at least one surfactant.

Furthermore, the invention provides a method for combating such pests, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a compound of the general formula I as defined herein or a salt thereof.

The invention provides in particular a method for protecting crops, including seeds, from attack or infestation by harmful insects, arachnids and/or nematodes, said method comprises contacting a crop with a pesticidally effective amount of at least one compound of formula I as defined herein or with a salt thereof.

The invention also provides a method for protecting non-living materials from attack or infestation by the aforementioned pests, which method comprises contacting the non-living material with a pesticidally effective amount of at least one compound of formula I as defined herein or with a salt thereof.

Suitable compounds of the general formula I encompass all possible stereoisomers (cis/trans isomers, enantiomers) which may occur and mixtures thereof. Stereoisomeric centers are e.g. the carbon atom of the C(R²)═C(R³) group and the C(R¹) moiety. The present invention provides both the pure enantiomes or diastereomers or mixtures thereof, the pure cis- and trans-isomers and the mixtures thereof. The compounds of the general formula I may also exist in the form of different tautomers if A or Ar carry amino or hydroxy groups. The invention comprises the single tautomers, if separable, as well as the tautomer mixtures.

Salts of the compounds of the formula I are preferably agriculturally acceptable salts. They can be formed in a customary method, e.g. by reacting the compound with an acid of the anion in question if the compound of formula I has a basic functionality or by reacting an acidic compound of formula I with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formula I with an acid of the corresponding anion, preferably of hydrochloric acid, hydrobromic acid, sulfuric acid, phosphoric acid or nitric acid.

The organic moieties mentioned in the above definitions of the variables are—like the term halogen—collective terms for individual listings of the individual group members. The prefix Cₙ-Cₘ indicates in each case the possible number of carbon atoms in the group.

“Halogen” or “halo” will be taken to mean fluoro, chloro, bromo and iodo.

The term C₁-C₄-alkylidene refers to a linear or branched alkanediyl group having 1 to 4 carbon atoms such as methylene (═CH₂), 1,2-ethandiyl (═CH₂CH₂), 1,1-ethandiyl (═CH(CH₃)), 1,2-propandiyl, 1,3-propandiyl, 2,2-propandiyl, 1,4-butandiyl and the like. According to the invention C₁-C₄-alkanediyl is unsubstituted or may carry 1, 2, 3 or 4 substituents selected from OH, ═O, halogen C₁-C₄ haloalkyl and C₁-C₄ alkoxy. The substituent ═O denotes a carbonyl group.

The terms “O—C₁-C₄-alkylidene” and “S—C₁-C₄-alkylidene”, respectively refer to a linear or branched alkanediyl group having 1 to 4 carbon atoms, where one valency is attached to the skeleton via an oxygen or sulfur atom, respectively.

The term “C₁-C₁₀-alkyl” as used herein (and also in C₁-C₁₀-alkylsulfinyl and C₁-C₁₀-alkylsulfonyl) refers to a branched or unbranched saturated hydrocarbon group having 1 to 10 carbon atoms, for example methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, 2-ethylhexyl, nonyl and decyl and their isomers. C₁-C₄-alkyl means for example methyl, ethyl, propyl, 1-methylethyl, butyl, 1-methylpropyl, 2-methylpropyl or 1,1-dimethylethyl.

The term “C₁-C₁₀-haloalkyl” as used herein (and also in C₁-C₁₀-haloalkylsulfinyl and C₁-C₁₀-haloalkylsulfonyl) refers to a straight-chain or branched alkyl group having 1 to 10 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like. The term C₁-C₁₀-haloalkyl in particular comprises C₁-C₂-fluoroalkyl, which is synonym with methyl or ethyl, wherein 1, 2, 3, 4 or 5 hydrogen atoms are substituted by fluorine atoms, such as fluoromethyl, difluoromethyl, trifluoromethyl, 1-fluoroethyl, 2-fluoroethyl, 2,2-difluoroethyl, 2,2,2-trifluoroethyl and pentafluoromethyl.

Similarly, “C₁-C₁₀-alkoxy” and “C₁-C₁₀-alkylthio” refer to straight-chain or branched alkyl groups having 1 to 10 carbon atoms (as mentioned above) bonded through oxygen or sulfur linkages, respectively, in any position in the alkyl group. Examples include C₁-C₄-alkoxy such as methoxy, ethoxy, propoxy, isopropoxy, butoxy, sec-butoxy, isobutoxy and tert-butoxy, further C₁-C₄-alkylthio such as methylthio, ethylthio, propylthio, isopropylthio, and n-butylthio.

Accordingly, the terms “C₁-C₁₀-haloalkoxy” and “C₁-C₁₀-haloalkylthio” refer to straight-chain or branched alkyl groups having 1 to 10 carbon atoms (as mentioned above) bonded through oxygen or sulfur linkages, respectively, in any position in the alkyl group, where some or all of the hydrogen atoms in these groups may be replaced by halogen atoms as mentioned above, for example C₁-C₂-haloalkoxy, such as chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy and pentafluoroethoxy, further C₁-C₂-haloalkylthio, such as chloromethylthio, bromomethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio and pentafluoroethylthio and the like. Similarly the terms C₁-C₂-fluoroalkoxy and C₁-C₂-fluoroalkylthio refer to C₁-C₂-fluoroalkyl which is bound to the remainder of the molecule via an oxygen atom or a sulfur atom, respectively.

The term “C₂-C₁₀-alkenyl” as used herein intends a branched or unbranched unsaturated hydrocarbon group having 2 to 10 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “C₂-C₁₀-haloalkenyl” as used herein intends a branched or unbranched unsaturated hydrocarbon group having 2 to 10 carbon atoms and a double bond in any position, where some or all of the hydrogen atoms in these groups may be replaced by halogen atoms as mentioned above.

Similarly, the term “C₂-C₁₀-alkenyloxy” as used herein intends a branched or unbranched unsaturated hydrocarbon group having 2 to 10 carbon atoms and a double bond in any position, the alkenyl group being bonded through oxygen linkage, in any position in the alkenyl group, for example ethenyloxy, propenyloxy and the like.

Accordingly, the term “C₂-C₁₀-haloalkenyloxy” as used herein intends a branched or unbranched unsaturated hydrocarbon group having 2 to 10 carbon atoms and a double bond in any position, the alkenyl group being bonded through oxygen linkage, in any position in the alkenyl group, where some or all of the hydrogen atoms in these groups may be replaced by halogen atoms as mentioned above.

The term “C₂-C₁₀-alkynyl” as used herein refers to a branched or unbranched unsaturated hydrocarbon group having 2 to 10 carbon atoms and containing at least one triple bond, such as ethynyl, propynyl, 1-butynyl, 2-butynyl, and the like.

The term “C₃-C₁₀-haloalkynyl” as used herein refers to a branched or unbranched unsaturated hydrocarbon group having 3 to 10 carbon atoms and containing at least one triple bond, where some or all of the hydrogen atoms in these groups may be replaced by halogen atoms as mentioned above, with the proviso that the halogen atom is not directly bound to the triple bond.

The term “C₂-C₁₀-alkynyloxy” as used herein refers to a branched or unbranched unsaturated hydrocarbon group having 2 to 10 carbon atoms and containing at least one triple bond, the alkynyl group being bonded through oxygen linkage in any position in the alkynyl group.

Similarly, the term “C₃-C₁₀-haloalkynyloxy” as used herein refers to a branched or unbranched unsaturated hydrocarbon group having 3 to 10 carbon atoms and containing at least one triple bond, the group being bonded through oxygen linkages in any position in the alkynyl group, where some or all of the hydrogen atoms in these group may be replaced by halogen atoms as mentioned above, with the proviso that the halogen atom is not directly bound to the triple bond.

The term “C₃-C₁₀-cycloalkyl” as used herein refers to a monocyclic 3- to 10-membered saturated carbon atom ring, e.g. cyclopropyl, cyclobutyl, cyclopentyl, cyclohexyl, cycloheptyl, cyclooctyl and cyclodecyl.

The term “C₃-C₁₀-halocycloalkyl” as used herein refers to a monocyclic 3- to 10-membered saturated carbon atom ring, e.g. cyclopropyl, cyclobutyl, cyclopentyl, cyclohexyl, cycloheptyl, cyclooctyl and cyclodecyl, where some or all of the hydrogen atoms in these groups may be replaced by halogen atoms as mentioned above, for example chloro-, dichloro- and trichlorocyclopropyl, fluoro-, difluoro- and trifluorocyclopropyl, chloro-, dichloro-, trichloro, tetrachloro-, pentachloro- and hexachlorocyclohexyl and the like.

The term “C₁-C₁₀-alkylcarbonyl” as used herein refers to C₁-C₁₀-alkyl which is bound to the remainder of the molecule via a carbonyl group. Examples include CO—CH₃, CO—C₂H₅, CO—CH₂—C₂H₅, CO—CH(CH₃)₂, n-butylcarbonyl, CO—CH(CH₃)—C₂H₅, CO—CH₂CH(CH₃)₂, CO—C(CH₃)₃, n-pentylcarbonyl, 1-methylbutylcarbonyl, 2-methylbutylcarbonyl, 3-methylbutylcarbonyl, 2,2-dimethylpropylcarbonyl, 1-ethylpropylcarbonyl, n-hexylcarbonyl, 1,1-dimethylpropylcarbonyl, 1,2-dimethylpropylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-I-methylpropylcarbonyl or 1-ethyl-2-methylpropylcarbonyl.

The term “C₁-C₁₀-alkoxycarbonyl” as used herein refers to C₁-C₁₀-alkoxy which is bound to the remainder of the molecule via a carbonyl group. Examples include CO—OCH₃, CO—OC₂H₅, CO—OCH₂—C₂H₅, CO—OCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “halo-C₁-C₁₀-alkoxycarbonyl” as used herein refers to C₁-C₁₀-haloalkoxy which is bound to the remainder of the molecule via a carbonyl group.

The terms “hydroxy-C₁-C₁₀-alkyl”, “C₁-C₁₀-alkoxy-C₁-C₁₀-alkyl”, “halo-C₁-C₁₀-alkoxy-C₁-C₁₀-alkyl”, “C₁-C₁₀-alkoxycarbonyl-C₁-C₁₀-alkyl”, “halo-C₁-C₁₀-alkoxycarbonyl-C₁-C₁₀-alkyl” as used herein, refer to C₁-C₁₀-alkyl, as defined herein, in particular to methyl, ethyl, 1-propyl or 2-propyl, which is substituted by one radical selected from hydroxy, C₁-C₁₀-alkoxy, C₁-C₁₀-haloalkoxy, C₁-C₁₀-alkoxycarbonyl or C₁-C₁₀-haloalkoxycarbonyl.

The term “5- or 6-membered heterocyclic radical with 1, 2, 3 or 4 heteroatoms which are selected, independently of one another, from O, N and S” comprises monocyclic 5- or 6-membered heteroaromatic rings and nonaromatic saturated or partially unsaturated 5- or 6-membered mono-heterocycles, which carry 1, 2, 3, or 4 heteroatoms as ring members. The heterocyclic radical may be attached to the remainder of the molecule via a carbon ring member or via a nitrogen ring member.

Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, dioxanyl, thiopyranyl, dihydrothiopyranyl, tetrahydrothiopyranyl, morpholinyl, thiazinyl and the like.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

With respect to the use according to the invention of the compounds of formula I, particular preference is given to the following meanings of the substituents, in each case on their own or in combination:

Preference is given to compounds of formula I, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be unsubstituted or substituted as described above. In particular the aformentioned radicals are unsubstituted or substituted by 1, 2 or 3 radicals R^{a} as defined above.

Preferred radicals R^{a} comprise halogen, CN, C₁-C₄-alkyl, C₁-C₄-alkoxy, C₁-C₄-haloalkoxy, C₁-C₄-alkylthio and C₁-C₄-haloalkyl, in particular F, Cl, methyl, methoxy, ethoxy, methylthio, difluoromethyl, trifluoromethyl, 2,2,2-trifluoroethyl, trifluoromethoxy and difluoromethoxy.

More preference is given to compounds of formula I, wherein A is a cyclic radical selected from phenyl, thienyl, furyl, isoxazolyl, pyrazolyl, imidazolyl, pyrazinyl, pyrimidinyl and pyridyl, in particular from pyridyl, thienyl and phenyl, where the cyclic radical may be substituted by 1, 2 or 3 substituents R^{a} which are as defined above and which are preferably selected, independently of one another, from halogen, C₁-C₄-alkyl, C₁-C₄-alkoxy, C₁-C₄-haloalkoxy, C₁-C₄-alkylthio and C,-C₄-haloalkyl, in particular from F, Cl, methyl, methoxy, ethoxy, methylthio, difluoromethyl, trifluoromethyl, 2,2,2-trifluoroethyl, trifluoromethoxy and difluoromethoxy.

Examples of preferred radicals A comprise:
2-thienyl, 3-bromothien-2-yl, 4-bromothien-2-yl, 5-bromothien-2-yl, 4,5-dibromothien-2-yl, 3-chlorothien-2-yl, 4-chlorothien-2-yl, 5-chlorothien-2-yl, 3-chloro-4-methylthien-2-yl, 3-methylthien-2-yl, 4-methylthien-2-yl, 5-methylthien-2-yl, 3-methoxythien-2-yl, 3-methoxy-5-(trifluoromethyl)thien-2-yl, 3-ethoxy-5-(trifluoromethyl)thien-2-yl, 2-bromo-4,5-dimethylthien-2-yl,
pyridin-2-yl, 6-methylthiopyridin-2-yl, 3-fluoropyridin-2-yl, 3-methoxypyridin-2-yl, 6-methylpyridin-2-yl, 5-nitropyridin-2-yl, 3-fluoropyridin-2-yl, 6-bromopyridin-2-yl, 6-chloropyridin-2-yl, 4-chloropyridin-2-yl, 3,6-dichloropyridin-2-yl, 6-fluoropyridin-2-yl, 5-(trifluoromethyl)pyridin-2-yl, 4-(trifluoromethyl)pyridin-2-yl, 6-(trifluoromethyl)pyridin-2-yl, 6-hydroxypyridin-2-yl, 6-methoxypyridin-2-yl, 3-hydroxypyridin-2-yl, 6-phenylpyridin-2-yl, pyridin-3-yl, 6-chloropyridin-3-yl, 2,6-dichloropyridin-3-yl, 5,6-dichloropyridin-3-yl, 2,6-dichloro-4-methylpyridin-3-yl, 6-fluoropyridin-3-yl, 6-bromopyridin-3-yl, 5-bromopyridin-3-yl, 2-phenoxypyridin-3-yl, 1-methyl-2-hydroxypyridin-3-yl, pyridin-4-yl, 2-chloropyridin-4-yl, 2,6-dichloropyridin-4-yl,
phenyl, 2-fluorophenyl, 3-fluorophenyl, 4-fluorophenyl, 2,4-difluorophenyl, 2,5-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 2-chloro-6-fluorophenyl, 2-chloro-4-fluorophenyl, 1,2,3,4,5-pentafluorophenyl, 2-bromophenyl, 3-bromophenyl, 4-bromophenyl, 5-bromo-2-chlorophenyl, 4-chloro-2-methylphenyl, 3-fluoro-4-methylphenyl, 2-methylphenyl, 3-methylphenyl, 4-methylphenyl, 2,6-dimethylphenyl, 2-(trifluoromethyl)phenyl, 3-(trifluoromethyl)phenyl, 4-(trifluoromethyl)phenyl, 2-fluoro-4-(trifluoromethyl)phenyl, 3,5-bis(trifluoromethyl)phenyl, 4-tert-butyl-phenyl, 2-methoxyphenyl, 3-methoxyphenyl, 4-methoxyphenyl, 3,4-dimethoxyphenyl, 3,4,5-trimethoxyphenyl, 2-ethoxyphenyl, 3-ethoxyphenyl, 4-ethoxyphenyl, 2-methoxy-3-(methylthio)phenyl, 2-nitrophenyl, 3-nitrophenyl, 4-nitrophenyl, 2-hydroxyphenyl, 3-hydroxyphenyl, 4-hydroxyphenyl, 4-hydroxy-3-methoxyphenyl, 2-fluoro-4-hydroxyphenyl, 2-aminophenyl, 3-aminophenyl, 4-aminophenyl, 6-amino-2-fluorophenyl, 4-(dimethylamino)phenyl, 4-phenylphenyl, 4-(methylsulfonyl)-2-nitrophenyl,
5-methylpyrazin-2-yl, 3-aminopyrazin-2-yl,
2-furyl, 5-bromofuran-2-yl, 2-methylfuran-3-yl
5-isoxazolyl, 3-methylisoxazolyl, 3-(1-methylethyl)isoxazolyl, 5-(chloromethyl)isoxazolyl, 5-(hydroxymethyl)isoxazolyl,
1,3-bis(1,1,1-trifluoroeth-2-yl)pyrazol-4-yl, 1-methyl-3-(trifluoromethyl)pyrazol-5-yl,
3-methylimidazol-2-yl, 4-methylimididazol-5-yl,
4-amino-2-methylpyrimidin-2-yl, 6-hydroxy-2-methylpyrimidin-4-yl,

The cyclic radical may also be napthyl, 1,2,3,4-tetrahydroisochinolin-3-yl, 2-benzothienyl, benzo[1,3]-dioxol-5-yl, indolyl or benzimidazolyl, where the cyclic radical may be unsubstituted or substituted as described above. In particular the aformentioned radicals are unsubstituted or substituted by 1, 2 or 3 radicals R^{a} as defined above. Examples of these radicals A comprise:
3-hydroxynapth-2-yl, 2-benzothienyl, 3-chloro-2-benzothienyl, 1-naphthyl, 2-naphthyl, 3-indolyl, 2-benzimidazolyl, 6,7-dimethoxy-3-methyl-1,2,3,4-tetrahydroisochinolinyl.

Another embodiment of the invention relates to compounds of the formula I wherein A is a 5- or 6-membered heterocyclic radical with 1 to 4 heteroatoms as ring members which are selected, independently of one another, from O, N and S and may also have a carbonyl group as ring member, and may have 1, 2, 3, 4 or 5 substituents R^{a} as defined above.

A very preferred embodiment of the invention relates to compounds of the formula I, wherein A is thienyl, in particular 2-thienyl which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-alkylthio, C₁-C₄-haloalkoxy and C₁-C₄-haloalkyl, in particular from F, Cl, Br, methyl, methoxy, difluoromethyl, trifluoromethyl, 2,2,2-trifluoroethyl, trifluoromethoxy and difluoromethoxy.

Another very preferred embodiment of the invention relates to compounds of the formula I, wherein A is furyl, in particular 2-furyl which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-alkylthio, C₁-C₄-haloalkoxy and C₁-C₄-haloalkyl, in particular from F, Cl, Br, methyl, methoxy, difluoromethyl, trifluoromethyl, 2,2,2-trifluoroethyl, trifluoromethoxy and difluoromethoxy.

Another very preferred embodiment of the invention relates to compounds of the formula I, wherein A is pyridyl, in particular 2- or 3-pyridyl, more preferably 2-pyridyl which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-alkylthio, C₁-C₄-haloalkoxy and C₁-C₄-haloalkyl, in particular from F, Cl, Br, methyl, methoxy, difluoromethyl, trifluoromethyl, 2,2,2-trifluoroethyl, trifluoromethoxy and difluoromethoxy.

A further very preferred embodiment of the invention relates to compounds of the formula I, wherein A is phenyl, which is unsubstituted or substituted by 1, 2 or 3 radicals R^{a} as defined above, the radicals R^{a} being preferably selected, independently of one another, from halogen, C₁-C₄-alkyl, C₁-C₄-alkoxy, C₁-C₄-haloalkoxy, C₁-C₄-alkylthio and C₁-C₄-haloalkyl, in particular from F, Cl, Br, methyl, methoxy, ethoxy, methylthio, difluoromethyl, trifluoromethyl, 2,2,2-trifluoroethyl, trifluoromethoxy and difluoromethoxy.

X is preferably C═O.

Q is preferably selected from the group consisting of a single bond, —CH₂—, —CH₂—CH₂—, —O—CH₂—, —O—CH₂—CH₂—, O—CH₂—CH₂—CH₂—, —NH—CH₂—CH₂—, —NH—C(O)—CH₂—, —S—CH₂—, —S—CH₂—CH₂—, —O—CH(CH₃)— and —S—CH(CH₃)—, in particular from a single bond, —CH₂—, —O—CH₂—, —S—CH₂—, —O—CH(CH₃)— and —S—CH(CH₃)—. The heteroatom of Q is preferably attached to A. More preferably Q is a single bond or —CH₂—.

Preference is also given to compounds of the formula I, wherein the moiety A-Q is C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy, in particular from ═O, OH and C₁-C₂-alkoxy. Preferred examples of the moiety A-Q include methyl, ethyl, n-propyl, n-butyl, n-pentyl, n-hexyl, n-heptyl, 4-hydroxybutyl, 5-hydroxypentyl, 2,2-dimethoxyethyl, 2,2-diethoxyethyl, methoxycarbonylmethyl or ethoxycarbonylmethyl, especially methyl.

In one embodiment of this invention R¹ is H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₂-C₁₀-haloalkynyl. In this embodiment R¹ is preferably hydrogen, C₁-C₁₀-alkyl, C₁-C₁₀-haloalkyl, more preferably hydrogen, C₁-C₄-alkyl or C₁-C₄-haloalkyl, and especially hydrogen. In a further embodiment of this invention R¹ is selected from the group consisting of phenyl and a 5- or 6-membered heterocyclic radical with 1 to 4 heteroatoms which are selected, independently of one another, from O, N and S, it being possible for phenyl and the 5- or 6-membered heterocyclic radical to be unsubstituted or substituted by 1, 2, 3, 4 or 5 substituents R^{a} as defined above. In this embodiment preference is given to compounds I, wherein R¹ is pyridyl, in particular 3-pyridyl, C₃-C₁₀-cycloalkyl, phenyl and more preferably C₃-C₆-cyclopropyl,

R² is preferably selected from hydrogen, halogen and C₁-C₄-alkyl, in particular from hydrogen, fluorine chlorine, bromine, methyl or ethyl.

R³ is preferably selected from hydrogen, halogen and C₁-C₄-alkyl, with hydrogen being more preferred.

R⁵ is preferably selected from hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, in particular from hydrogen and C₁-C₄-alkyl.

In a further embodiment R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, R⁷R⁸N—CO— and benzyl wherein benzyl may be substituted by 1, 2, 3, 4 or 5 substituents R^{b} as defined above. In this embodiment R⁶ is preferably hydrogen, C₁-C₄-alkyl, C₁-C₄-haloalkyl, C₁-C₄-alkylsulfonyl, C₁-C₄-alkylcarbonyl, C₁-C₄-alkylsulfonyl or C₁-C₄-haloalkylsulfonyl, in particular hydrogen.

Preference is also given to compounds of the formula I, wherein R⁶ is C₁-C₁₀-alkylthio, C₁-C₁₀-haloalkylthio, C₁-C₁₀-alkylcarbonyl, where the alkyl moiety of alkylcarbonyl is substituted by 1, 2 or 3 substituents selected from the group consisting of C₁-C₁₀-alkylthio, phenylthio, phenyl and phenoxy, C₂-C₁₀-alkenylcarbonyl, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above. In this embodiment R⁶ is preferably C₁-C₄-alkylcarbonyl, wherein the alkyl moiety of alkylcarbonyl is substituted by a radical selected from phenyl, C₁-C₄-alkylthio, phenylthio and phenoxy, such as phenoxymethylcarbonyl, 2-methylsulfanylethylcarbonyl and phenylsulfanylmethylcarbonyl or benzoyl. Preference is also given to compounds of the formula I, wherein R⁶ is a benzoyl or a radical of the formula R^{6a}—C(O)—, wherein R^{6a} is C₁-C₂-alkoxycarbonyl, phenoxy, a 5- or 6-membered aromatic heterocyclic radical with 1 or 2 heteroatoms which are selected, independently of one another, from O, N and S, it being possible for the 5- or 6-membered heterocyclic radical to be unsubstituted or substituted by 1or 2 substituents R^{a} as defined above. Example of preferred radicals R^{6a} are phenoxy, 3-isoxazolyl, 5-isoxazolyl, 2-thienyl and 2-furyl and methoxycarbonyl.

Ar is preferably phenyl, which is unsubstituted or substituted by 1, 2, 3 or 4, in particular 1, 2 or 3 radicals R^{c} as defined above. A skilled person will appreciate that in case of R⁴ being different from hydrogen, R⁴ is one of the 1 to 4 radicals R^{c} as defined above or R⁴ together with R² is the aformentioned bivalent radical.

Another embodiment of the invention relates to compounds I, wherein Ar is an aromatic radical selected from phenyl, naphthyl, pyridyl, pyrimidyl, furyl and thienyl and wherein two radicals R^{c} which are bound to adjacent carbon atoms may form a O—CH₂—O moiety, where in said moiety 1 or 2 hydrogen atoms may be replaced by a radical R^{b} as defined above.

In one embodiment the radical R^{c} is halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above. Preferably the radicals R^{c} are selected, independently of one another, from halogen, CN, C₁-C₄-alkyl, C₁-C₄-alkoxy, C₁-C₄-haloalkoxy and C₁-C₄-haloalkyl, more preferably F, Cl, CN, C₁-C₃-alkoxy, in particular methoxy, trifluoromethyl, difluoromethyl, trifluoromethoxy, difluoromethoxy and methyl. In another embodiment the radical R^{c} is C₁-C₁₀-alkylcarbonyl.

A further very preferred embodiment of the invention relates to compounds of the formula I, wherein Ar is furyl, in particular 2-furyl which is unsubstituted or substituted by 1 radical R^{c} as defined above or thienyl, in particular 2-thienyl, which may be unsubstituted or substituted by 1 radical R^{c} as defined above.

In a preferred embodiment of the invention the radical R² in formula I is a monovalent radical, i.e. R² and R⁴ together do not form a bivalent radical. In this embodiment R² is preferably selected from hydrogen, halogen, C₁-C₄-alkyl, C₁-C₄-alkoxy, C₁-C₄-haloalkoxy and C₁-C₄-haloalkyl. More preferably R² is hydrogen, fluorine, chlorine bromine, methyl or ethyl. In this embodiment R⁴ is hydrogen or a radical R^{c} as defined above, in particular hydrogen. In this embodiment Ar in formula I is preferably phenyl, which is unsubstituted or substituted by 1, 2, 3 or 4, in particular 1, 2 or 3 radicals R^{c} as defined above.

Another embodiment of the invention relates to compounds of the formula I, wherein R⁴ together with R² is a bivalent radical Y as defined above and which is preferably selected from O, S, CH═N, O—CH₂, O—C(O) or NH—C(O), where in the last 4 moieties either the carbon atom or the heteroatom is attached to the phenyl ring, in particular from O, S, O—CH₂, and O—C(O), where in the two last moieties the heteroatom is attached to the phenyl ring. More preferably R⁴ and R² together are an oxygen atom or O—C(O) in particular O. In this embodiment Ar in formula I is preferably phenyl, which is unsubstituted or substituted by 1, 2 or 3, in particular 0, 1 or 2 radicals R^{c} as defined above.

Apart from that, R^{b} is preferably selected from halogen, C₁-C₄-alkyl, C₁-C₄-alkoxy, C₁-C₄-haloalkoxy and C₁-C₄-haloalkyl.

If present, R⁷, R⁸ are preferably selected independently from one another from hydrogen and C₁-C₄-alkyl.

If present, R⁹ is preferably selected from hydrogen, C₁-C₄-alkyl and C₁-C₄-alkylcarbonyl, in particular hydrogen.

If present, R¹⁰ is preferably selected from hydrogen, halogen and C₁-C₄-alkyl, in particular hydrogen.

If present, R¹¹, R¹² are preferably selected independently from one another from hydrogen and C₁-C₄-alkyl, in particular hydrogen.

If present, R¹³ is preferably selected from hydrogen and C₁-C₄-alkyl, in particular hydrogen.

A very preferred embodiment of the invention relates to compounds of the general formula Ia:

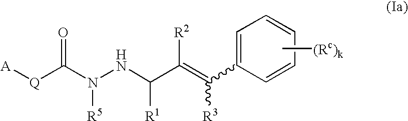

wherein k is 0, 1, 2 or 3, and wherein A, Q, R¹, R², R³, R⁵ and R^{c} are as defined above. Amongst the compounds Ia those are preferred, wherein Q, A, R¹, R², R³, R⁵ and R^{c} have the meanings given as preferred.

Examples of compounds Ia are given in the following tables 1 to 180:

Table 1:

Compounds of the formula Ia, wherein R¹ is H, R³is H, R²is H, R⁵ is H and k is 0 (i.e. (R^{c})ₖ is absent) and wherein Q and A are given in table A;

Table 2:

Compounds of the formula Ia, wherein R¹ is H, R³is H, R²is H, R⁵ is H and (R^{c})ₖ is 4-fluoro and wherein Q and A are given in table A;

Table 3:

Compounds of the formula Ia, wherein R¹ is H, R³ is H, R²is H, R⁵is H and (R^{c})ₖ is 4-chloro and wherein Q and A are given table A;

Table 4:

Compounds of the formula Ia, wherein R¹ is H, R³is H, R²is H, R⁵ is H and (R^{c})ₖ is 3-fluoro and wherein Q and A are given in table A;

Table 5:

Compounds of the formula Ia, wherein R¹ is H, R³is H, R²is H, R⁵ is H and (R^{c})ₖ is 3-difluoromethoxy and wherein Q and A are given in table A;

Table 6:

Compounds of the formula Ia, wherein R¹ is H, R³is H, R²is H, R⁶is H and (R^{c})ₖ is 3-trifluoromethyl and wherein Q and A are given in table A;

Table 7:

Compounds of the formula Ia, wherein R¹ is H, R³is H, R²is H, R⁵ is H and (R^{c})ₖ is 4-fluoro-3-trifluoromethyl and wherein Q and A are given in table A;

Table 8:

Compounds of the formula Ia, wherein R¹ is H, R³is H, R²is H, R⁵ is H and (R^{c})ₖ is 4-methoxy-3-trifluoromethyl and wherein Q and A are in given table A;

Table 9:

Compounds of the formula Ia, wherein R¹ is H, R³ is H, R² is H, R⁵ is H and (R^{c})ₖ is 4-methylthio-3-trifluoromethyl and wherein Q and A are given in table A;

Table 10:

Compounds of the formula Ia, wherein R¹ is H, R³is H, R² is H, R⁵ is H and (R^{c})ₖ is 2-F and wherein Q and A are given in table A;

Table 11:

Compounds of the formula Ia, wherein R¹ is H, R³is H, R² is H, R⁵ is H and (R^{c})ₖ is 2-CH₃ and wherein Q and A are given in table A;

Table 12:

Compounds of the formula Ia, wherein R¹ is H, R³is H, R²is H, R⁵ is H and (R^{c})ₖ is 2-OCH₃ and wherein Q and A are given in table A;

Table 13:

Compounds of the formula Ia, wherein R¹ is H, R³is H, R² is H, R⁵ is H and (R^{c})ₖ is 2-CF₃ and wherein Q and A are given in table A;

Table 14:

Compounds of the formula Ia, wherein R¹ is H, R³is H, R² is H, R⁵ is H and (R^{c})ₖ is 4-OCH₃ and wherein Q and A are given in table A;

Table 15:

Compounds of the formula Ia, wherein R¹ is H, R³is H, R² is H, R⁵ is H and (R^{c})ₖ is 2-OCHF₂ and wherein Q and A are given in table A;

Table 16:

Compounds of the formula Ia, wherein R¹ is H, R³is H, R² is F, R⁵ is H and k is 0 (i.e. (R^{c})ₖ is absent) and wherein Q and A are given in table A;

Table 17:

Compounds of the formula Ia, wherein R¹ is H, R³ is H, R² is F, R⁵ is H and (R^{c})ₖ is 4-fluoro and wherein Q and A are given in table A;

Table 18:

Compounds of the formula Ia, wherein R¹ is H, R³ is H, R² is F, R⁵ is H and (R^{c})ₖ is 4-chloro and wherein Q and A are given in table A;

Table 19:

Compounds of the formula Ia, wherein R¹ is H, R³ is H, R² is F, R⁵ is H and (R^{c})ₖ is 3-fluoro and wherein Q and A are given in table A;

Table 20:

Compounds of the formula Ia, wherein R¹ is H, R³ is H, R² is F, R⁵ is H and (R^{c})ₖ is 3-difluoromethoxy and wherein Q and A are given in table A;

Table 21:

Compounds of the formula Ia, wherein R¹ is H, R³ is H, R² is F, R⁵ is H and (R^{c})ₖ is 3-trifluoromethyl and wherein Q and A are given in table A;

Table 22:

Compounds of the formula Ia, wherein R¹ is H, R³ is H, R² is F, R⁵ is H and (R^{c})ₖ is 4-fluoro-3-trifluoromethyl and wherein Q and A are given in table A;

Table 23:

Compounds of the formula Ia, wherein R¹ is H, R³ is H, R² is F, R⁵ is H and (R^{c})ₖ is 4-methoxy-3-trifluoromethyl and wherein Q and A are given in table A;

Table 24:

Compounds of the formula Ia, wherein R¹ is H, R³ is H, R² is F, R⁵ is H and (R^{c})ₖ is 4-methylthio-3-trifluoromethyl and wherein Q and A are given in table A;

Table 25:

Compounds of the formula Ia, wherein R¹ is H, R³is H, R² is F, R⁵ is H and (R^{c})ₖ is 2-F and wherein Q and A are given in table A;

Table 26:

Compounds of the formula Ia, wherein R¹ is H, R³ is H, R² is F, R⁵ is H and (R^{c})ₖ is 2-CH₃ and wherein Q and A are given in table A;

Table 27:

Compounds of the formula Ia, wherein R¹ is H, R³ is H, R² is F, R⁵ is H and (R^{c})ₖ is 2-OCH₃ and wherein Q and A are given in table A;

Table 28:

Compounds of the formula Ia, wherein R¹ is H, R³ is H, R²is F, R⁵ is H and (R^{c})ₖ is 2-CF₃ and wherein Q and A are given in table A;

Table 29:

Compounds of the formula Ia, wherein R¹ is H, R³ is H, R² is F, R⁵ is H and (R^{c})ₖ is 4-OCH₃ and wherein Q and A are given in table A;

Table 30:

Compounds of the formula Ia, wherein R¹ is H, R³ is H, R² is F, R⁵ is H and (R^{c})ₖ is 2-OCHF₂ and wherein Q and A are given in table A;

Table 31:

Compounds of the formula Ia, wherein R¹ is H, R³ is H, R² is Cl, R⁵ is H and k is 0 (i.e. (R^{c})ₖ is absent) and wherein Q and A are given in table A;

Table 32:

Compounds of the formula Ia, wherein R¹ is H, R³ is H, R² is Cl, R⁵ is H and (R^{c})ₖ is 4-fluoro and wherein Q and A are given in table A;

Table 33:

Compounds of the formula Ia, wherein R¹ is H, and R³ is H, R² is Cl, R⁵ is H and (R^{c})ₖ is 4-chloro and wherein Q and A are given in table A;

Table 34:

Compounds of the formula Ia, wherein R¹ is H, and R³ is H, R² is Cl, R⁵ is H and (R^{c})ₖ is 3-fluoro and wherein Q and A are given in table A;

Table 35:

Compounds of the formula Ia, wherein R¹ is H, R³ is H, R² is Cl, R⁵ is H and (R^{c})ₖ is 3-difluoromethoxy and wherein Q and A are given in table A;

Table 36:

Compounds of the formula Ia, wherein R¹ is H, R³ is H, R² is Cl, R⁵ is H and (R^{c})ₖ is 3-trifluoromethyl and wherein Q and A are given in table A;

Table 37:

Compounds of the formula Ia, wherein R¹ is H, R³ is H, R² is Cl, R⁵ is H and (R^{c})ₖ is 4-fluoro-3-trifluoromethyl and wherein Q and A are given in table A;

Table 38:

Compounds of the formula Ia, wherein R¹ is H, R³ is H, R² is Cl, R⁵ is H and (R^{c})ₖ is 4-methoxy-3-trifluoromethyl and wherein Q and A are given in table A;

Table 39:

Compounds of the formula Ia, wherein R¹ is H, R³ is H, R² is Cl, R⁵ is H and (R^{c})ₖ is 4-methylthio-3-trifluoromethyl and wherein Q and A are given in table A;

Table 40:

Compounds of the formula Ia, wherein R¹ is H, R³ is H, R² is Cl, R⁵ is H and (R^{c})ₖ is 2-F and wherein Q and A are given in table A;

Table 41:

Compounds of the formula Ia, wherein R¹ is H, R³ is H, R² is Cl, R⁵ is H and (R^{c})ₖ is 2-CH₃ and wherein Q and A are given in table A;

Table 42:

Compounds of the formula Ia, wherein R¹ is H, R³ is H, R² is Cl, R⁵ is H and (R^{c})ₖ is 2-OCH₃ and wherein Q and A are given in table A;

Table 43:

Compounds of the formula Ia, wherein R¹ is H, R³ is H, R² is Cl, R⁵ is H and (R^{c})ₖ is 2-CF₃ and wherein Q and A are given in table A;

Table 44:

Compounds of the formula Ia, wherein R¹ is H, R³ is H, R² is Cl, R⁵ is H and (R^{c})ₖ is 4-OCH₃ and wherein Q and A are given in table A;

Table 45:

Compounds of the formula Ia, wherein R¹ is H, R³ is H, R² is Cl, R⁵ is H and (R^{c})ₖ is 2-OCHF₂ and wherein Q and A are given in table A;

Table 46:

Compounds of the formula Ia, wherein R¹ is H, R³ is H, R² is Br, R⁵ is H and k is 0 (i.e. (R^{c})ₖ is absent) and wherein Q and A are given in table A;

Table 47:

Compounds of the formula Ia, wherein R¹ is H, R³is H, R² is Br, R⁵ is H and (R^{c})ₖ is 4-fluoro and wherein Q and A are given in table A;

Table 48:

Compounds of the formula Ia, wherein R¹ is H, R³ is H, R² is Br, R⁵ is H and (R^{c})ₖ is 4-chloro and wherein Q and A are given in table A;

Table 49:

Compounds of the formula Ia, wherein R¹ is H, R³ is H, R² is B, R⁵ is H r and (R^{c})ₖ is 3-fluoro and wherein Q and A are given in table A;

Table 50:

Compounds of the formula Ia, wherein R¹ is H, R³ is H, R² is Br, R⁵ is H and (R^{c})ₖ is 3-difluoromethoxy and wherein Q and A are given in table A;

Table 51:

Compounds of the formula Ia, wherein R¹ is H, R³ is H, R² is Br, R⁵ is H and (R^{c})ₖ is 3-trifluoromethyl and wherein Q and A are given in table A;

Table 52:

Compounds of the formula Ia, wherein R¹ is H, R³ is H, R² is Br, R⁵ is H and (R^{c})ₖ is 4-fluoro-3-trifluoromethyl and wherein Q and A are given in table A;

Table 53:

Compounds of the formula Ia, wherein R¹ is H, R³ is H, R² is Br, R⁵ is H and (R^{c})ₖ is 4-methoxy-3-trifluoromethyl and wherein Q and A are given in table A;

Table 54:

Compounds of the formula Ia, wherein R¹ is H, R³ is H, R² is Br, R⁵ is H and (R^{c})ₖ is 4-methylthio-3-trifluoromethyl and wherein Q and A are given in table A;

Table 55:

Compounds of the formula Ia, wherein R¹ is H, R³ is H, R² is Br, R⁵ is H and (R^{c})ₖ is 2-F and wherein Q and A are given in table A;

Table 56:

Compounds of the formula Ia, wherein R¹ is H, R³ is H, R² is Br, R⁵ is H and (R^{c})ₖ is 2-CH₃ and wherein Q and A are given in table A;

Table 57:

Compounds of the formula Ia, wherein R¹ is H, R³ is H, R² is Br, R⁵ is H and (R^{c})ₖ is 2-OCH₃ and wherein Q and A are given in table A;

Table 58:

Compounds of the formula Ia, wherein R¹ is H, R³ is H, R² is Br, R⁵ is H and (R^{c})ₖ is 2-CF₃ and wherein Q and A are given in table A;

Table 59:

Compounds of the formula Ia, wherein R¹ is H, R³ is H, R² is Br, R⁵ is H and (R^{c})ₖ is 4-OCH₃ and wherein Q and A are given in table A;

Table 60:

Compounds of the formula Ia, wherein R¹ is H, R³ is H, R²is Br, R⁵ is H and (R^{c})ₖ is 2-OCHF₂ and wherein Q and A are given in table A;

Table 61:

Compounds of the formula Ia, wherein R¹ is H, R³ is H, R² is CH₃, R⁵ is H and k is 0 (i.e. (R^{c})ₖ is absent) and wherein Q and A are given in table A;

Table 62:

Compounds of the formula Ia, wherein R¹ is H, R³ is H, R² is CH₃, R⁵ is H and (R^{c})ₖ is 4-fluoro and wherein Q and A are given in table A;

Table 63:

Compounds of the formula Ia, wherein R¹ is H, R³ is H, R² is CH₃, R⁵ is H and (R^{c})ₖ is 4-chloro and wherein Q and A are given in table A;

Table 64:

Compounds of the formula Ia, wherein R¹ is H, R³ is H, R² is CH₃, R⁵ is H and (R^{c})ₖ is 3-fluoro and wherein Q and A are given in table A;

Table 65:

Compounds of the formula Ia, wherein R¹ is H, R³ is H, R² is CH₃, R⁵ is H and (R^{c})ₖ is 3-difluoromethoxy and wherein Q and A are given in table A;

Table 66:

Compounds of the formula Ia, wherein R¹ is H, R³ is H, R² is CH₃, R⁵ is H and (R^{c})ₖ is 3-trifluoromethyl and wherein Q and A are given in table A;

Table 67:

Compounds of the formula Ia, wherein R¹ is H, R³ is H, R² is CH₃, R⁵ is H and (R^{c})ₖ is 4-fluoro-3-trifluoromethyl and wherein Q and A are given in table A;

Table 68:

Compounds of the formula Ia, wherein R¹ is H, R³ is H, R² is CH₃, R⁵ is H and (R^{c})ₖ is 4-methoxy-3-trifluoromethyl and wherein Q and A are given in table A; and

Table 69:

Compounds of the formula Ia, wherein R¹ is H, R³ is H, R² is CH₃, R⁵ is H and (R^{c})ₖ is 4-methylthio-3-trifluoromethyl and wherein Q and A are in given table A.

Table 70:

Compounds of the formula Ia, wherein R¹ is H, R³ is H, R² is CH₃, R⁵ is H and (R^{c})ₖ is 2-F and wherein Q and A are given in table A;

Table 71:

Compounds of the formula Ia, wherein R¹ is H, R³ is H, R² is CH₃, R⁵ is H and (R^{c})ₖ is 2-CH₃ and wherein Q and A are given in table A;

Table 72:

Compounds of the formula Ia, wherein R¹ is H, R³ is H, R² is CH₃, R⁵ is H and (R^{c})ₖ is 2-OCH₃ and wherein Q and A are given in table A;

Table 73:

Compounds of the formula Ia, wherein R¹ is H, R³ is H, R² is CH₃, R⁵ is H and (R^{c})ₖ is 2-CF₃ and wherein Q and A are given in table A;

Table 74:

Compounds of the formula Ia, wherein R¹ is H, R³ is H, R² is CH₃, R⁵ is H and (R^{c})ₖ is 4-OCH₃ and wherein Q and A are given in table A;

Table 75:

Compounds of the formula Ia, wherein R¹ is H, R³ is H, R² is CH₃, R⁵ is H and (R^{c})ₖ is 2-OCHF₂ and wherein Q and A are given in table A;

Table 76:

Compounds of the formula Ia, wherein R¹ is H, R³ is H, R² is CH₂CH₃, R⁵ is H and k is 0 (i.e. (R^{c})ₖ is absent) and wherein Q and A are given in table A;

Table 77:

Compounds of the formula Ia, wherein R¹ is H, R³ is H, R² is CH₂CH₃, R⁵ is H and (R^{c})ₖ is 4-fluoro and wherein Q and A are given in table A;

Table 78:

Compounds of the formula Ia, wherein R¹ is H, R³ is H, R² is CH₂CH₃, R⁵ is H and (R^{c})ₖ is 4-chloro and wherein Q and A are given in table A;

Table 79:

Compounds of the formula Ia, wherein R¹ is H, R³ is H, R² is CH₂CH₃, R⁵ is H and (R^{c})ₖ is 3-fluoro and wherein Q and A are given in table A;

Table 80:

Compounds of the formula Ia, wherein R¹ is H, R³ is H, R² is CH₂CH₃, R⁵ is H and (R^{c})ₖ is 3-difluoromethoxy and wherein Q and A are given in table A;

Table 81:

Compounds of the formula Ia, wherein R¹ is H, R³ is H, R² is CH₂CH₃, R⁵ is H and (R^{c})ₖ is 3-trifluoromethyl and wherein Q and A are given in table A;

Table 82:

Compounds of the formula Ia, wherein R¹ is H, R³ is H, R² is CH₂CH₃, R⁵ is H and (R^{c})ₖ is 4-fluoro-3-trifluoromethyl and wherein Q and A are given in table A;

Table 83:

Compounds of the formula Ia, wherein R¹ is H, R³ is H, R² is CH₂CH₃, R⁵ is H and (R^{c})ₖ is 4-methoxy-3-trifluoromethyl and wherein Q and A are given in table A;

Table 84:

Compounds of the formula Ia, wherein R¹ is H, R³ is H, R² is CH₂CH₃, R⁵ is H and (R^{c})ₖ is 4-methylthio-3-trifluoromethyl and wherein Q and A are given in table A;

Table 85:

Compounds of the formula Ia, wherein R¹ is H, R³ is H, R² is CH₂CH₃, R⁵ is H and (R^{c})ₖ is 2-F and wherein Q and A are given in table A;

Table 86:

Compounds of the formula Ia, wherein R¹ is H, R³ is H, R² is CH₂CH₃, R⁵ is H and (R^{c})ₖ is 2-CH₃ and wherein Q and A are given in table A;

Table 87:

Compounds of the formula Ia, wherein R¹ is H, R³ is H, R² is CH₂CH₃, R⁵ is H and (R^{c})ₖ is 2-OCH₃ and wherein Q and A are given in table A;

Table 88:

Compounds of the formula Ia, wherein R¹ is H, R³ is H, R² is CH₂CH₃, R⁵ is H and (R^{c})ₖ is2-CF₃ and wherein Q and A are given in table A;

Table 89:

Compounds of the formula Ia, wherein R¹ is H, R³ is H, R² is CH₂CH₃, R⁵ is H and (R^{c})ₖ is 4-OCH₃ and wherein Q and A are given in table A;.

Table 90:

Compounds of the formula Ia, wherein R¹ is H, R³ is H, R² is CH₂CH₃, R⁵ is H and (R^{c})ₖ is 2-OCHF₂ and wherein Q and A are given in table A;

Table 91 to 180:

Compounds of the formula Ia, wherein R¹, R², R³ and (R^{c})ₖ are as defined in one of the tables 1 to 90, Q and A are given in table A and R⁵ is methyl instead of hydrogen.

TABLE A

| | A | Q |
|---|---|---|
| 1 | 2-thienyl | — |
| 2 | 3-bromothien-2-yl | — |
| 3 | 4-bromothien-2-yl | — |
| 4 | 5-bromothien-2-yl | — |
| 5 | 4,5-dibromthien-2-yl | — |
| 6 | 3-chlorothien-2-yl | — |
| 7 | 4-chlorothien-2-yl | — |
| 8 | 5-chlorothien-2-yl | — |
| 9 | 3-chloro-4-methylthien-2-yl | — |
| 10 | 3-methylthien-2-yl | — |
| 11 | 4-methylthien-2-yl | — |
| 12 | 5-methylthien-2-yl | — |
| 13 | 3-methoxythien-2-yl | — |
| 14 | 3-methoxy-5-(trifluoromethyl)thien-2-yl | — |
| 15 | 3-ethoxy-5-(trifluoromethyl)thien-2-yl | — |
| 16 | 2-bromo-4,5-dimethylthien-2-yl | — |
| 17 | pyridin-2-yl | — |
| 18 | 6-methylthiopyridin-2-yl | — |
| 19 | 3-fluoropyridin-2-yl | — |
| 20 | 3-methoxypyridin-2-yl | — |
| 21 | 6-methylpyridin-2-yl | — |
| 22 | 5-nitropyridin-2-yl | — |
| 23 | 3-fluoropyridin-2-yl | — |
| 24 | 6-bromopyridin-2-yl | — |
| 25 | 6-chloropyridin-2-yl | — |
| 26 | 4-chloropyridin-2-yl | — |
| 27 | 3,6-dichloropyridin-2-yl | — |
| 28 | 6-fluoropyridin-2-yl | — |
| 29 | 5-(trifluoromethyl)pyridin-2-yl | — |
| 30 | 4-(trifluoromethyl)pyridin-2-yl | — |
| 31 | 6-(trifluoromethyl)pyridin-2-yl | — |
| 32 | 6-hydroxypyridin-2-yl | — |
| 33 | 6-methoxypyridin-2-yl | — |
| 34 | 3-hydroxypyridin-2-yl | — |
| 35 | 6-phenylpyridin-2-yl | — |
| 36 | pyridin-3-yl | — |
| 37 | 6-chloropyridin-3-yl | — |
| 38 | 2,6-dichloropyridin-3-yl | — |
| 39 | 5,6-dichloropyridin-3-yl | — |
| 40 | 2,6-dichloro-4-methylpyridin-3-yl | — |
| 41 | 6-fluoropyridin-3-yl | — |
| 42 | 6-bromopyridin-3-yl | — |
| 43 | 5-bromopyridin-3-yl | — |
| 44 | 2-phenoxypyridin-3-yl | — |
| 45 | 1-methyl-2-hydroxypyridin-3-yl | — |
| 46 | pyridin-4-yl | — |
| 47 | 2-chloropyridin-4-yl | — |
| 48 | 2,6-dichloropyridin-4-yl | — |
| 49 | phenyl | — |
| 50 | 2-fluorophenyl | — |
| 51 | 3-fluorophenyl | — |
| 52 | 4-fluorophenyl | — |
| 53 | 2,4-difluorophenyl | — |
| 54 | 2,5-difluorophenyl | — |
| 55 | 2-chlorophenyl | — |
| 56 | 3-chlorophenyl | — |
| 57 | 4-chlorophenyl | — |
| 58 | 2,3-dichlorophenyl | — |
| 59 | 2,4-dichlorophenyl | — |
| 60 | 2,5-dichlorophenyl | — |
| 61 | 2,6-dichlorophenyl | — |
| 62 | 2-chloro-6-fluorophenyl | — |
| 63 | 2-chloro-4-fluorophenyl | — |
| 64 | 1,2,3,4,5-pentafluorophenyl | — |
| 65 | 2-bromophenyl | — |
| 66 | 3-bromophenyl | — |
| 67 | 4-bromophenyl | — |
| 68 | 5-bromo-2-chlorophenyl | — |
| 69 | 4-chloro-2-methylphenyl | — |
| 70 | 3-fluoro-4-methylphenyl | — |
| 71 | 2-methylphenyl | — |
| 72 | 3-methylphenyl | — |
| 73 | 4-methylphenyl | — |
| 74 | 2,6-dimethylphenyl | — |
| 75 | 2-(trifluoromethyl)phenyl | — |
| 76 | 3-(trifluoromethyl)phenyl | — |
| 77 | 4-(trifluoromethyl)phenyl | — |
| 78 | 2-fluoro-4-(trifluoromethyl)phenyl | — |
| 79 | 3,5-bis(trifluoromethyl)phenyl | — |
| 80 | 4-tert.-butyl-phenyl | — |
| 81 | 2-methoxyphenyl | — |
| 82 | 3-methoxyphenyl | — |
| 83 | 4-methoxyphenyl | — |
| 84 | 3,4-dimethoxyphenyl | — |
| 85 | 3,4,5-trimethoxyphenyl | — |
| 86 | 2-ethoxyphenyl | — |
| 87 | 3-ethoxyphenyl | — |
| 88 | 4-ethoxyphenyl | — |
| 89 | 2-methoxy-3-(methylthio)phenyl | — |
| 90 | 2-nitrophenyl | — |
| 91 | 3-nitrophenyl | — |
| 92 | 4-nitrophenyl | — |
| 93 | 2-hydroxyphenyl | — |
| 94 | 3-hydroxyphenyl | — |
| 95 | 4-hydroxyphenyl | — |
| 96 | 4-hydroxy-3-methoxyphenyl | — |
| 97 | 2-fluoro-4-hydroxyphenyl | — |
| 98 | 2-aminophenyl | — |
| 99 | 3-aminophenyl | — |
| 100 | 4-aminophenyl | — |
| 101 | 6-amino-2-fluorophenyl | — |
| 102 | 4-(dimethylamino)phenyl | — |
| 103 | 4-phenylphenyl | — |
| 104 | 4-(methylsulfonyl)-2-nitrophenyl | — |
| 105 | 5-methylpyrazin-2-yl | — |
| 106 | 3-aminopyrazin-2-yl | — |
| 107 | 2-furyl | — |
| 108 | 5-bromofuran-2-yl | — |
| 109 | 2-methylfuran-3-yl | — |
| 110 | 5-isoxazolyl | — |
| 111 | 3-methylisoxazolyl | — |
| 112 | 3-(1-methylethyl)isoxazolyl | — |
| 113 | 5-(chloromethyl)isoxazolyl | — |
| 114 | 5-(hydroxymethyl)isoxazolyl | — |
| 115 | 1,3-bis(1,1,1-trifluoroeth-2-yl)pyrazol-4-yl | — |
| 116 | 1-methyl-3-(trifluoromethyl)pyrazol-5-yl | — |
| 117 | 3-methylimidazol-2-yl | — |
| 118 | 4-methylimididazol-5-yl | — |
| 119 | 4-amino-2-methylpyrimidin-2-yl | — |
| 120 | 6-hydroxy-2-methylpyrimidin-4-yl | — |
| 121 | 3-hydroxynapth-2-yl | — |
| 122 | 2-benzothienyl | — |
| 123 | 3-chloro-2-benzothienyl | — |
| 124 | 1-naphthyl | — |
| 125 | 2-naphthyl | — |
| 126 | 3-indolyl | — |
| 127 | 2-benzimidazolyl | — |
| 128 | 6,7-dimethoxy-3-methyl-1,2,3,4- | — |
| | tetrahydroisochinolin-3-yl | |
| 129 | 2-thienyl | CH₂ |
| 130 | 3-bromothien-2-yl | CH₂ |
| 131 | 4-bromothien-2-yl | CH₂ |
| 132 | 5-bromothien-2-yl | CH₂ |
| 133 | 4,5-dibromthien-2-yl | CH₂ |
| 134 | 3-chlorothien-2-yl | CH₂ |
| 135 | 4-chlorothien-2-yl | CH₂ |
| 136 | 5-chlorothien-2-yl | CH₂ |
| 137 | 3-chloro-4-methylthien-2-yl | CH₂ |
| 138 | 3-methylthien-2-yl | CH₂ |
| 139 | 4-methylthien-2-yl | CH₂ |
| 140 | 5-methylthien-2-yl | CH₂ |
| 141 | 3-methoxythien-2-yl | CH₂ |
| 142 | 3-methoxy-5-(trifluoromethyl)thien-2-yl | CH₂ |
| 143 | 3-ethoxy-5-(trifluoromethyl)thien-2-yl | CH₂ |
| 144 | 2-bromo-4,5-dimethylthien-2-yl | CH₂ |
| 145 | pyridin-2-yl | CH₂ |
| 146 | 6-methylthiopyridin-2-yl | CH₂ |
| 147 | 3-fluoropyridin-2-yl | CH₂ |
| 148 | 3-methoxypyridin-2-yl | CH₂ |
| 149 | 6-methylpyridin-2-yl | CH₂ |
| 150 | 5-nitropyridin-2-yl | CH₂ |
| 151 | 3-fluoropyridin-2-yl | CH₂ |
| 152 | 6-bromopyridin-2-yl | CH₂ |
| 153 | 6-chloropyridin-2-yl | CH₂ |
| 154 | 4-chloropyridin-2-yl | CH₂ |
| 155 | 3,6-dichloropyridin-2-yl | CH₂ |
| 156 | 6-fluoropyridin-2-yl | CH₂ |
| 157 | 5-(trifluoromethyl)pyridin-2-yl | CH₂ |
| 158 | 4-(trifluoromethyl)pyridin-2-yl | CH₂ |
| 159 | 6-(trifluoromethyl)pyridin-2-yl | CH₂ |
| 160 | 6-hydroxypyridin-2-yl | CH₂ |
| 161 | 6-methoxypyridin-2-yl | CH₂ |
| 162 | 3-hydroxypyridin-2-yl | CH₂ |
| 163 | 6-phenylpyridin-2-yl | CH₂ |
| 164 | pyridin-3-yl | CH₂ |
| 165 | 6-chloropyridin-3-yl | CH₂ |
| 166 | 2,6-dichloropyridin-3-yl | CH₂ |
| 167 | 5,6-dichloropyridin-3-yl | CH₂ |
| 168 | 2,6-dichloro-4-methylpyridin-3-yl | CH₂ |
| 169 | 6-fluoropyridin-3-yl | CH₂ |
| 170 | 6-bromopyridin-3-yl | CH₂ |
| 171 | 5-bromopyridin-3-yl | CH₂ |
| 172 | 2-phenoxypyridin-3-yl | CH₂ |
| 173 | 1-methyl-2-hydroxypyridin-3-yl | CH₂ |
| 174 | pyridin-4-yl | CH₂ |
| 175 | 2-chloropyridin-4-yl | CH₂ |
| 176 | 2,6-dichloropyridin-4-yl | CH₂ |
| 177 | phenyl | CH₂ |
| 178 | 2-fluorophenyl | CH₂ |
| 179 | 3-fluorophenyl | CH₂ |
| 180 | 4-fluorophenyl | CH₂ |
| 181 | 2,4-difluorophenyl | CH₂ |
| 182 | 2,5-difluorophenyl | CH₂ |
| 183 | 2-chlorophenyl | CH₂ |
| 184 | 3-chlorophenyl | CH₂ |
| 185 | 4-chlorophenyl | CH₂ |
| 186 | 2,3-dichlorophenyl | CH₂ |
| 187 | 2,4-dichlorophenyl | CH₂ |
| 188 | 2,5-dichlorophenyl | CH₂ |
| 189 | 2,6-dichlorophenyl | CH₂ |
| 190 | 2-chloro-6-fluorophenyl | CH₂ |
| 191 | 2-chloro-4-fluorophenyl | CH₂ |
| 192 | 1,2,3,4,5-pentafluorophenyl | CH₂ |
| 193 | 2-bromophenyl | CH₂ |
| 194 | 3-bromophenyl | CH₂ |
| 195 | 4-bromophenyl | CH₂ |
| 196 | 5-bromo-2-chlorophenyl | CH₂ |
| 197 | 4-chloro-2-methylphenyl | CH₂ |
| 198 | 3-fluoro-4-methylphenyl | CH₂ |
| 199 | 2-methylphenyl | CH₂ |
| 200 | 3-methylphenyl | CH₂ |
| 201 | 4-methylphenyl | CH₂ |
| 202 | 2,6-dimethylphenyl | CH₂ |
| 203 | 2-(trifluoromethyl)phenyl | CH₂ |
| 204 | 3-(trifluoromethyl)phenyl | CH₂ |
| 205 | 4-(trifluoromethyl)phenyl | CH₂ |
| 206 | 2-fluoro-4-(trifluoromethyl)phenyl | CH₂ |
| 207 | 3,5-bis(trifluoromethyl)phenyl | CH₂ |
| 208 | 4-tert.-butyl-phenyl | CH₂ |
| 209 | 2-methoxyphenyl | CH₂ |
| 210 | 3-methoxyphenyl | CH₂ |
| 211 | 4-methoxyphenyl | CH₂ |
| 212 | 3,4-dimethoxyphenyl | CH₂ |
| 213 | 3,4,5-trimethoxyphenyl | CH₂ |
| 214 | 2-ethoxyphenyl | CH₂ |
| 215 | 3-ethoxyphenyl | CH₂ |
| 216 | 4-ethoxyphenyl | CH₂ |
| 217 | 2-methoxy-3-(methylthio)phenyl | CH₂ |
| 218 | 2-nitrophenyl | CH₂ |
| 219 | 3-nitrophenyl | CH₂ |
| 220 | 4-nitrophenyl | CH₂ |
| 221 | 2-hydroxyphenyl | CH₂ |
| 222 | 3-hydroxyphenyl | CH₂ |
| 223 | 4-hydroxyphenyl | CH₂ |
| 224 | 4-hydroxy-3-methoxyphenyl | CH₂ |
| 225 | 2-fluoro-4-hydroxyphenyl | CH₂ |
| 226 | 2-aminophenyl | CH₂ |
| 227 | 3-aminophenyl | CH₂ |
| 228 | 4-aminophenyl | CH₂ |
| 229 | 6-amino-2-fluorophenyl | CH₂ |
| 230 | 4-(dimethylamino)phenyl | CH₂ |
| 231 | 4-phenylphenyl | CH₂ |
| 232 | 4-(methylsulfonyl)-2-nitrophenyl | CH₂ |
| 233 | 5-methylpyrazin-2-yl | CH₂ |
| 234 | 3-aminopyrazin-2-yl | CH₂ |
| 235 | 2-furyl | CH₂ |
| 236 | 5-bromofuran-2-yl | CH₂ |
| 237 | 2-methylfuran-3-yl | CH₂ |
| 238 | 5-isoxazolyl | CH₂ |
| 239 | 3-methylisoxazolyl | CH₂ |
| 240 | 3-(1-methylethyl)isoxazolyl | CH₂ |
| 241 | 5-(chloromethyl)isoxazolyl | CH₂ |
| 242 | 5-(hydroxymethyl)isoxazolyl | CH₂ |
| 243 | 1,3-bis(1,1,1-trifluoroeth-2-yl)pyrazol-4-yl | CH₂ |
| 244 | 1-methyl-3-(trifluoromethyl)pyrazol-5-yl | CH₂ |
| 245 | 3-methylimidazol-2-yl | CH₂ |
| 246 | 4-methylimididazol-5-yl | CH₂ |
| 247 | 4-amino-2-methylpyrimidin-2-yl | CH₂ |
| 248 | 6-hydroxy-2-methylpyrimidin-4-yl | CH₂ |
| 249 | 3-hydroxynapth-2-yl | CH₂ |
| 250 | 2-benzothienyl | CH₂ |
| 251 | 3-chloro-2-benzothienyl | CH₂ |
| 252 | 1-naphthyl | CH₂ |
| 253 | 2-naphthyl | CH₂ |
| 254 | 3-indolyl | CH₂ |
| 255 | 2-benzimidazolyl | CH₂ |
| 256 | 6,7-dimethoxy-3-methyl-1,2,3,4- | CH₂ |
| | tetrahydroisochinolin-3-yl | |
| 257 | 2-thienyl | CH₂CH₂ |
| 258 | 3-bromothien-2-yl | CH₂CH₂ |
| 259 | 4-bromothien-2-yl | CH₂CH₂ |
| 260 | 5-bromothien-2-yl | CH₂CH₂ |
| 261 | 4,5-dibromthien-2-yl | CH₂CH₂ |
| 262 | 3-chlorothien-2-yl | CH₂CH₂ |
| 263 | 4-chlorothien-2-yl | CH₂CH₂ |
| 264 | 5-chlorothien-2-yl | CH₂CH₂ |
| 265 | 3-chloro-4-methylthien-2-yl | CH₂CH₂ |
| 266 | 3-methylthien-2-yl | CH₂CH₂ |
| 267 | 4-methylthien-2-yl | CH₂CH₂ |
| 268 | 5-methylthien-2-yl | CH₂CH₂ |
| 269 | 3-methoxythien-2-yl | CH₂CH₂ |
| 270 | 3-methoxy-5-(trifluoromethyl)thien-2-yl | CH₂CH₂ |
| 271 | 3-ethoxy-5-(trifluoromethyl)thien-2-yl | CH₂CH₂ |
| 272 | 2-bromo-4,5-dimethylthien-2-yl | CH₂CH₂ |
| 273 | pyridin-2-yl | CH₂CH₂ |
| 274 | 6-methylthiopyridin-2-yl | CH₂CH₂ |
| 275 | 3-fluoropyridin-2-yl | CH₂CH₂ |
| 276 | 3-methoxypyridin-2-yl | CH₂CH₂ |
| 277 | 6-methylpyridin-2-yl | CH₂CH₂ |
| 278 | 5-nitropyridin-2-yl | CH₂CH₂ |
| 279 | 3-fluoropyridin-2-yl | CH₂CH₂ |
| 280 | 6-bromopyridin-2-yl | CH₂CH₂ |
| 281 | 6-chloropyridin-2-yl | CH₂CH₂ |
| 282 | 4-chloropyridin-2-yl | CH₂CH₂ |
| 283 | 3,6-dichloropyridin-2-yl | CH₂CH₂ |
| 284 | 6-fluoropyridin-2-yl | CH₂CH₂ |
| 285 | 5-(trifluoromethyl)pyridin-2-yl | CH₂CH₂ |
| 286 | 4-(trifluoromethyl)pyridin-2-yl | CH₂CH₂ |
| 287 | 6-(trifluoromethyl)pyridin-2-yl | CH₂CH₂ |
| 288 | 6-hydroxypyridin-2-yl | CH₂CH₂ |
| 289 | 6-methoxypyridin-2-yl | CH₂CH₂ |
| 290 | 3-hydroxypyridin-2-yl | CH₂CH₂ |
| 291 | 6-phenylpyridin-2-yl | CH₂CH₂ |
| 292 | pyridin-3-yl | CH₂CH₂ |
| 293 | 6-chloropyridin-3-yl | CH₂CH₂ |
| 294 | 2,6-dichloropyridin-3-yl | CH₂CH₂ |
| 295 | 5,6-dichloropyridin-3-yl | CH₂CH₂ |
| 296 | 2,6-dichloro-4-methylpyridin-3-yl | CH₂CH₂ |
| 297 | 6-fluoropyridin-3-yl | CH₂CH₂ |
| 298 | 6-bromopyridin-3-yl | CH₂CH₂ |
| 299 | 5-bromopyridin-3-yl | CH₂CH₂ |
| 300 | 2-phenoxypyridin-3-yl | CH₂CH₂ |
| 301 | 1-methyl-2-hydroxypyridin-3-yl | CH₂CH₂ |
| 302 | pyridin-4-yl | CH₂CH₂ |
| 303 | 2-chloropyridin-4-yl | CH₂CH₂ |
| 304 | 2,6-dichloropyridin-4-yl | CH₂CH₂ |
| 305 | phenyl | CH₂CH₂ |
| 306 | 2-fluorophenyl | CH₂CH₂ |
| 307 | 3-fluorophenyl | CH₂CH₂ |
| 308 | 4-fluorophenyl | CH₂CH₂ |
| 309 | 2,4-difluorophenyl | CH₂CH₂ |
| 310 | 2,5-difluorophenyl | CH₂CH₂ |
| 311 | 2-chlorophenyl | CH₂CH₂ |
| 312 | 3-chlorophenyl | CH₂CH₂ |
| 313 | 4-chlorophenyl | CH₂CH₂ |
| 314 | 2,3-dichlorophenyl | CH₂CH₂ |
| 315 | 2,4-dichlorophenyl | CH₂CH₂ |
| 316 | 2,5-dichlorophenyl | CH₂CH₂ |
| 317 | 2,6-dichlorophenyl | CH₂CH₂ |
| 318 | 2-chloro-6-fluorophenyl | CH₂CH₂ |
| 319 | 2-chloro-4-fluorophenyl | CH₂CH₂ |
| 320 | 1,2,3,4,5-pentafluorophenyl | CH₂CH₂ |
| 321 | 2-bromophenyl | CH₂CH₂ |
| 322 | 3-bromophenyl | CH₂CH₂ |
| 323 | 4-bromophenyl | CH₂CH₂ |
| 324 | 5-bromo-2-chlorophenyl | CH₂CH₂ |
| 325 | 4-chloro-2-methylphenyl | CH₂CH₂ |
| 326 | 3-fluoro-4-methylphenyl | CH₂CH₂ |
| 327 | 2-methylphenyl | CH₂CH₂ |
| 328 | 3-methylphenyl | CH₂CH₂ |
| 329 | 4-methylphenyl | CH₂CH₂ |
| 330 | 2,6-dimethylphenyl | CH₂CH₂ |
| 331 | 2-(trifluoromethyl)phenyl | CH₂CH₂ |
| 332 | 3-(trifluoromethyl)phenyl | CH₂CH₂ |
| 333 | 4-(trifluoromethyl)phenyl | CH₂CH₂ |
| 334 | 2-fluoro-4-(trifluoromethyl)phenyl | CH₂CH₂ |
| 335 | 3,5-bis(trifluoromethyl)phenyl | CH₂CH₂ |
| 336 | 4-tert.-butyl-phenyl | CH₂CH₂ |
| 337 | 2-methoxyphenyl | CH₂CH₂ |
| 338 | 3-methoxyphenyl | CH₂CH₂ |
| 339 | 4-methoxyphenyl | CH₂CH₂ |
| 340 | 3,4-dimethoxyphenyl | CH₂CH₂ |
| 341 | 3,4,5-trimethoxyphenyl | CH₂CH₂ |
| 342 | 2-ethoxyphenyl | CH₂CH₂ |
| 343 | 3-ethoxyphenyl | CH₂CH₂ |
| 344 | 4-ethoxyphenyl | CH₂CH₂ |
| 345 | 2-methoxy-3-(methylthio)phenyl | CH₂CH₂ |
| 346 | 2-nitrophenyl | CH₂CH₂ |
| 347 | 3-nitrophenyl | CH₂CH₂ |
| 348 | 4-nitrophenyl | CH₂CH₂ |
| 349 | 2-hydroxyphenyl | CH₂CH₂ |
| 350 | 3-hydroxyphenyl | CH₂CH₂ |
| 351 | 4-hydroxyphenyl | CH₂CH₂ |
| 352 | 4-hydroxy-3-methoxyphenyl | CH₂CH₂ |
| 353 | 2-fluoro-4-hydroxyphenyl | CH₂CH₂ |
| 354 | 2-aminophenyl | CH₂CH₂ |
| 355 | 3-aminophenyl | CH₂CH₂ |
| 356 | 4-aminophenyl | CH₂CH₂ |
| 357 | 6-amino-2-fluorophenyl | CH₂CH₂ |
| 358 | 4-(dimethylamino)phenyl | CH₂CH₂ |
| 359 | 4-phenylphenyl | CH₂CH₂ |
| 360 | 4-(methylsulfonyl)-2-nitrophenyl | CH₂CH₂ |
| 361 | 5-methylpyrazin-2-yl | CH₂CH₂ |
| 362 | 3-aminopyrazin-2-yl | CH₂CH₂ |
| 363 | 2-furyl | CH₂CH₂ |
| 364 | 5-bromofuran-2-yl | CH₂CH₂ |
| 365 | 2-methylfuran-3-yl | CH₂CH₂ |
| 366 | 5-isoxazolyl | CH₂CH₂ |
| 367 | 3-methylisoxazolyl | CH₂CH₂ |
| 368 | 3-(1-methylethyl)isoxazolyl | CH₂CH₂ |
| 369 | 5-(chloromethyl)isoxazolyl | CH₂CH₂ |
| 370 | 5-(hydroxymethyl)isoxazolyl | CH₂CH₂ |
| 371 | 1,3-bis(1,1,1-trifluoroeth-2-yl)pyrazol-4-yl | CH₂CH₂ |
| 372 | 1-methyl-3-(trifluoromethyl)pyrazol-5-yl | CH₂CH₂ |
| 373 | 3-methylimidazol-2-yl | CH₂CH₂ |
| 374 | 4-methylimididazol-5-yl | CH₂CH₂ |
| 375 | 4-amino-2-methylpyrimidin-2-yl | CH₂CH₂ |
| 376 | 6-hydroxy-2-methylpyrimidin-4-yl | CH₂CH₂ |
| 377 | 3-hydroxynapth-2-yl | CH₂CH₂ |
| 378 | 2-benzothienyl | CH₂CH₂ |
| 379 | 3-chloro-2-benzothienyl | CH₂CH₂ |
| 380 | 1-naphthyl | CH₂CH₂ |
| 381 | 2-naphthyl | CH₂CH₂ |
| 382 | 3-indolyl | CH₂CH₂ |
| 383 | 2-benzimidazolyl | CH₂CH₂ |
| 384 | 6,7-dimethoxy-3-methyl-1,2,3,4- | CH₂CH₂ |
| | tetrahydroisochinolin-3-yl | |
| 385 | 2-thienyl | OCH₂ |
| 386 | 3-bromothien-2-yl | OCH₂ |
| 387 | 4-bromothien-2-yl | OCH₂ |
| 388 | 5-bromothien-2-yl | OCH₂ |
| 389 | 4,5-dibromthien-2-yl | OCH₂ |
| 390 | 3-chlorothien-2-yl | OCH₂ |
| 391 | 4-chlorothien-2-yl | OCH₂ |
| 392 | 5-chlorothien-2-yl | OCH₂ |
| 393 | 3-chloro-4-methylthien-2-yl | OCH₂ |
| 394 | 3-methylthien-2-yl | OCH₂ |
| 395 | 4-methylthien-2-yl | OCH₂ |
| 396 | 5-methylthien-2-yl | OCH₂ |
| 397 | 3-methoxythien-2-yl | OCH₂ |
| 398 | 3-methoxy-5-(trifluoromethyl)thien-2-yl | OCH₂ |
| 399 | 3-ethoxy-5-(trifluoromethyl)thien-2-yl | OCH₂ |
| 400 | 2-bromo-4,5-dimethylthien-2-yl | OCH₂ |
| 401 | pyridin-2-yl | OCH₂ |
| 402 | 6-methylthiopyridin-2-yl | OCH₂ |
| 403 | 3-fluoropyridin-2-yl | OCH₂ |
| 404 | 3-methoxypyridin-2-yl | OCH₂ |
| 405 | 6-methylpyridin-2-yl | OCH₂ |
| 406 | 5-nitropyridin-2-yl | OCH₂ |
| 407 | 3-fluoropyridin-2-yl | OCH₂ |
| 408 | 6-bromopyridin-2-yl | OCH₂ |
| 409 | 6-chloropyridin-2-yl | OCH₂ |
| 410 | 4-chloropyridin-2-yl | OCH₂ |
| 411 | 3,6-dichloropyridin-2-yl | OCH₂ |
| 412 | 6-fluoropyridin-2-yl | OCH₂ |
| 413 | 5-(trifluoromethyl)pyridin-2-yl | OCH₂ |
| 414 | 4-(trifluoromethyl)pyridin-2-yl | OCH₂ |
| 415 | 6-(trifluoromethyl)pyridin-2-yl | OCH₂ |
| 416 | 6-hydroxypyridin-2-yl | OCH₂ |
| 417 | 6-methoxypyridin-2-yl | OCH₂ |
| 418 | 3-hydroxypyridin-2-yl | OCH₂ |
| 419 | 6-phenylpyridin-2-yl | OCH₂ |
| 420 | pyridin-3-yl | OCH₂ |
| 421 | 6-chloropyridin-3-yl | OCH₂ |
| 422 | 2,6-dichloropyridin-3-yl | OCH₂ |
| 423 | 5,6-dichloropyridin-3-yl | OCH₂ |
| 424 | 2,6-dichloro-4-methylpyridin-3-yl | OCH₂ |
| 425 | 6-fluoropyridin-3-yl | OCH₂ |
| 426 | 6-bromopyridin-3-yl | OCH₂ |
| 427 | 5-bromopyridin-3-yl | OCH₂ |
| 428 | 2-phenoxypyridin-3-yl | OCH₂ |
| 429 | 1-methyl-2-hydroxypyridin-3-yl | OCH₂ |
| 430 | pyridin-4-yl | OCH₂ |
| 431 | 2-chloropyridin-4-yl | OCH₂ |
| 432 | 2,6-dichloropyridin-4-yl | OCH₂ |
| 433 | phenyl | OCH₂ |
| 434 | 2-fluorophenyl | OCH₂ |
| 435 | 3-fluorophenyl | OCH₂ |
| 436 | 4-fluorophenyl | OCH₂ |
| 437 | 2,4-difluorophenyl | OCH₂ |
| 438 | 2,5-difluorophenyl | OCH₂ |
| 439 | 2-chlorophenyl | OCH₂ |
| 440 | 3-chlorophenyl | OCH₂ |
| 441 | 4-chlorophenyl | OCH₂ |
| 442 | 2,3-dichlorophenyl | OCH₂ |
| 443 | 2,4-dichlorophenyl | OCH₂ |
| 444 | 2,5-dichlorophenyl | OCH₂ |
| 445 | 2,6-dichlorophenyl | OCH₂ |
| 446 | 2-chloro-6-fluorophenyl | OCH₂ |
| 447 | 2-chloro-4-fluorophenyl | OCH₂ |
| 448 | 1,2,3,4,5-pentafluorophenyl | OCH₂ |
| 449 | 2-bromophenyl | OCH₂ |
| 450 | 3-bromophenyl | OCH₂ |
| 451 | 4-bromophenyl | OCH₂ |
| 452 | 5-bromo-2-chlorophenyl | OCH₂ |
| 453 | 4-chloro-2-methylphenyl | OCH₂ |
| 454 | 3-fluoro-4-methylphenyl | OCH₂ |
| 455 | 2-methylphenyl | OCH₂ |
| 456 | 3-methylphenyl | OCH₂ |
| 457 | 4-methylphenyl | OCH₂ |
| 458 | 2,6-dimethylphenyl | OCH₂ |
| 459 | 2-(trifluoromethyl)phenyl | OCH₂ |
| 460 | 3-(trifluoromethyl)phenyl | OCH₂ |
| 461 | 4-(trifluoromethyl)phenyl | OCH₂ |
| 462 | 2-fluoro-4-(trifluoromethyl)phenyl | OCH₂ |
| 463 | 3,5-bis(trifluoromethyl)phenyl | OCH₂ |
| 464 | 4-tert.-butyl-phenyl | OCH₂ |
| 465 | 2-methoxyphenyl | OCH₂ |
| 466 | 3-methoxyphenyl | OCH₂ |
| 467 | 4-methoxyphenyl | OCH₂ |
| 468 | 3,4-dimethoxyphenyl | OCH₂ |
| 469 | 3,4,5-trimethoxyphenyl | OCH₂ |
| 470 | 2-ethoxyphenyl | OCH₂ |
| 471 | 3-ethoxyphenyl | OCH₂ |
| 472 | 4-ethoxyphenyl | OCH₂ |
| 473 | 2-methoxy-3-(methylthio)phenyl | OCH₂ |
| 474 | 2-nitrophenyl | OCH₂ |
| 475 | 3-nitrophenyl | OCH₂ |
| 476 | 4-nitrophenyl | OCH₂ |
| 477 | 2-hydroxyphenyl | OCH₂ |
| 478 | 3-hydroxyphenyl | OCH₂ |
| 479 | 4-hydroxyphenyl | OCH₂ |
| 480 | 4-hydroxy-3-methoxyphenyl | OCH₂ |
| 481 | 2-fluoro-4-hydroxyphenyl | OCH₂ |
| 482 | 2-aminophenyl | OCH₂ |
| 483 | 3-aminophenyl | OCH₂ |
| 484 | 4-aminophenyl | OCH₂ |
| 485 | 6-amino-2-fluorophenyl | OCH₂ |
| 486 | 4-(dimethylamino)phenyl | OCH₂ |
| 487 | 4-phenylphenyl | OCH₂ |
| 488 | 4-(methylsulfonyl)-2-nitrophenyl | OCH₂ |
| 489 | 5-methylpyrazin-2-yl | OCH₂ |
| 490 | 3-aminopyrazin-2-yl | OCH₂ |
| 491 | 2-furyl | OCH₂ |
| 492 | 5-bromofuran-2-yl | OCH₂ |
| 493 | 2-methylfuran-3-yl | OCH₂ |
| 494 | 5-isoxazolyl | OCH₂ |
| 495 | 3-methylisoxazolyl | OCH₂ |
| 496 | 3-(1-methylethyl)isoxazolyl | OCH₂ |
| 497 | 5-(chloromethyl)isoxazolyl | OCH₂ |
| 498 | 5-(hydroxymethyl)isoxazolyl | OCH₂ |
| 499 | 1,3-bis(1,1,1-trifluoroeth-2-yl)pyrazol-4-yl | OCH₂ |
| 500 | 1-methyl-3-(trifluoromethyl)pyrazol-5-yl | OCH₂ |
| 501 | 3-methylimidazol-2-yl | OCH₂ |
| 502 | 4-methylimididazol-5-yl | OCH₂ |
| 503 | 4-amino-2-methylpyrimidin-2-yl | OCH₂ |
| 504 | 6-hydroxy-2-methylpyrimidin-4-yl | OCH₂ |
| 505 | 3-hydroxynapth-2-yl | OCH₂ |
| 506 | 2-benzothienyl | OCH₂ |
| 507 | 3-chloro-2-benzothienyl | OCH₂ |
| 508 | 1-naphthyl | OCH₂ |
| 509 | 2-naphthyl | OCH₂ |
| 510 | 3-indolyl | OCH₂ |
| 511 | 2-benzimidazolyl | OCH₂ |
| 512 | 6,7-dimethoxy-3-methyl-1,2,3,4- | OCH₂ |
| | tetrahydroisochinolin-3-yl | |
| 513 | 2-thienyl | OCH(CH₃) |
| 514 | 3-bromothien-2-yl | OCH(CH₃) |
| 515 | 4-bromothien-2-yl | OCH(CH₃) |
| 516 | 5-bromothien-2-yl | OCH(CH₃) |
| 517 | 4,5-dibromthien-2-yl | OCH(CH₃) |
| 518 | 3-chlorothien-2-yl | OCH(CH₃) |
| 519 | 4-chlorothien-2-yl | OCH(CH₃) |
| 520 | 5-chlorothien-2-yl | OCH(CH₃) |
| 521 | 3-chloro-4-methylthien-2-yl | OCH(CH₃) |
| 522 | 3-methylthien-2-yl | OCH(CH₃) |
| 523 | 4-methylthien-2-yl | OCH(CH₃) |
| 524 | 5-methylthien-2-yl | OCH(CH₃) |
| 525 | 3-methoxythien-2-yl | OCH(CH₃) |
| 526 | 3-methoxy-5-(trifluoromethyl)thien-2-yl | OCH(CH₃) |
| 527 | 3-ethoxy-5-(trifluoromethyl)thien-2-yl | OCH(CH₃) |
| 528 | 2-bromo-4,5-dimethylthien-2-yl | OCH(CH₃) |
| 529 | pyridin-2-yl | OCH(CH₃) |
| 530 | 6-methylthiopyridin-2-yl | OCH(CH₃) |
| 531 | 3-fluoropyridin-2-yl | OCH(CH₃) |
| 532 | 3-methoxypyridin-2-yl | OCH(CH₃) |
| 533 | 6-methylpyridin-2-yl | OCH(CH₃) |
| 534 | 5-nitropyridin-2-yl | OCH(CH₃) |
| 535 | 3-fluoropyridin-2-yl | OCH(CH₃) |
| 536 | 6-bromopyridin-2-yl | OCH(CH₃) |
| 537 | 6-chloropyridin-2-yl | OCH(CH₃) |
| 538 | 4-chloropyridin-2-yl | OCH(CH₃) |
| 539 | 3,6-dichloropyridin-2-yl | OCH(CH₃) |
| 540 | 6-fluoropyridin-2-yl | OCH(CH₃) |
| 541 | 5-(trifluoromethyl)pyridin-2-yl | OCH(CH₃) |
| 542 | 4-(trifluoromethyl)pyridin-2-yl | OCH(CH₃) |
| 543 | 6-(trifluoromethyl)pyridin-2-yl | OCH(CH₃) |
| 544 | 6-hydroxypyridin-2-yl | OCH(CH₃) |
| 545 | 6-methoxypyridin-2-yl | OCH(CH₃) |
| 546 | 3-hydroxypyridin-2-yl | OCH(CH₃) |
| 547 | 6-phenylpyridin-2-yl | OCH(CH₃) |
| 548 | pyridin-3-yl | OCH(CH₃) |
| 549 | 6-chloropyridin-3-yl | OCH(CH₃) |
| 550 | 2,6-dichloropyridin-3-yl | OCH(CH₃) |
| 551 | 5,6-dichloropyridin-3-yl | OCH(CH₃) |
| 552 | 2,6-dichloro-4-methylpyridin-3-yl | OCH(CH₃) |
| 553 | 6-fluoropyridin-3-yl | OCH(CH₃) |
| 554 | 6-bromopyridin-3-yl | OCH(CH₃) |
| 555 | 5-bromopyridin-3-yl | OCH(CH₃) |
| 556 | 2-phenoxypyridin-3-yl | OCH(CH₃) |
| 557 | 1-methyl-2-hydroxypyridin-3-yl | OCH(CH₃) |
| 558 | pyridin-4-yl | OCH(CH₃) |
| 559 | 2-chloropyridin-4-yl | OCH(CH₃) |
| 560 | 2,6-dichloropyridin-4-yl | OCH(CH₃) |
| 561 | phenyl | OCH(CH₃) |
| 562 | 2-fluorophenyl | OCH(CH₃) |
| 563 | 3-fluorophenyl | OCH(CH₃) |
| 564 | 4-fluorophenyl | OCH(CH₃) |
| 565 | 2,4-difluorophenyl | OCH(CH₃) |
| 566 | 2,5-difluorophenyl | OCH(CH₃) |
| 567 | 2-chlorophenyl | OCH(CH₃) |
| 568 | 3-chlorophenyl | OCH(CH₃) |
| 569 | 4-chlorophenyl | OCH(CH₃) |
| 570 | 2,3-dichlorophenyl | OCH(CH₃) |
| 571 | 2,4-dichlorophenyl | OCH(CH₃) |
| 572 | 2,5-dichlorophenyl | OCH(CH₃) |
| 573 | 2,6-dichlorophenyl | OCH(CH₃) |
| 574 | 2-chloro-6-fluorophenyl | OCH(CH₃) |
| 575 | 2-chloro-4-fluorophenyl | OCH(CH₃) |
| 576 | 1,2,3,4,5-pentafluorophenyl | OCH(CH₃) |
| 577 | 2-bromophenyl | OCH(CH₃) |
| 578 | 3-bromophenyl | OCH(CH₃) |
| 579 | 4-bromophenyl | OCH(CH₃) |
| 580 | 5-bromo-2-chlorophenyl | OCH(CH₃) |
| 581 | 4-chloro-2-methylphenyl | OCH(CH₃) |
| 582 | 3-fluoro-4-methylphenyl | OCH(CH₃) |
| 583 | 2-methylphenyl | OCH(CH₃) |
| 584 | 3-methylphenyl | OCH(CH₃) |
| 585 | 4-methylphenyl | OCH(CH₃) |
| 586 | 2,6-dimethylphenyl | OCH(CH₃) |
| 587 | 2-(trifluoromethyl)phenyl | OCH(CH₃) |
| 588 | 3-(trifluoromethyl)phenyl | OCH(CH₃) |
| 589 | 4-(trifluoromethyl)phenyl | OCH(CH₃) |
| 590 | 2-fluoro-4-(trifluoromethyl)phenyl | OCH(CH₃) |
| 591 | 3,5-bis(trifluoromethyl)phenyl | OCH(CH₃) |
| 592 | 4-tert.-butyl-phenyl | OCH(CH₃) |
| 593 | 2-methoxyphenyl | OCH(CH₃) |
| 594 | 3-methoxyphenyl | OCH(CH₃) |
| 595 | 4-methoxyphenyl | OCH(CH₃) |
| 596 | 3,4-dimethoxyphenyl | OCH(CH₃) |
| 597 | 3,4,5-trimethoxyphenyl | OCH(CH₃) |
| 598 | 2-ethoxyphenyl | OCH(CH₃) |
| 599 | 3-ethoxyphenyl | OCH(CH₃) |
| 600 | 4-ethoxyphenyl | OCH(CH₃) |
| 601 | 2-methoxy-3-(methylthio)phenyl | OCH(CH₃) |
| 602 | 2-nitrophenyl | OCH(CH₃) |
| 603 | 3-nitrophenyl | OCH(CH₃) |
| 604 | 4-nitrophenyl | OCH(CH₃) |
| 605 | 2-hydroxyphenyl | OCH(CH₃) |
| 606 | 3-hydroxyphenyl | OCH(CH₃) |
| 607 | 4-hydroxyphenyl | OCH(CH₃) |
| 608 | 4-hydroxy-3-methoxyphenyl | OCH(CH₃) |
| 609 | 2-fluoro-4-hydroxyphenyl | OCH(CH₃) |
| 610 | 2-aminophenyl | OCH(CH₃) |
| 611 | 3-aminophenyl | OCH(CH₃) |
| 612 | 4-aminophenyl | OCH(CH₃) |
| 613 | 6-amino-2-fluorophenyl | OCH(CH₃) |
| 614 | 4-(dimethylamino)phenyl | OCH(CH₃) |
| 615 | 4-phenylphenyl | OCH(CH₃) |
| 616 | 4-(methylsulfonyl)-2-nitrophenyl | OCH(CH₃) |
| 617 | 5-methylpyrazin-2-yl | OCH(CH₃) |
| 618 | 3-aminopyrazin-2-yl | OCH(CH₃) |
| 619 | 2-furyl | OCH(CH₃) |
| 620 | 5-bromofuran-2-yl | OCH(CH₃) |
| 621 | 2-methylfuran-3-yl | OCH(CH₃) |
| 622 | 5-isoxazolyl | OCH(CH₃) |
| 623 | 3-methylisoxazolyl | OCH(CH₃) |
| 624 | 3-(1-methylethyl)isoxazolyl | OCH(CH₃) |
| 625 | 5-(chloromethyl)isoxazolyl | OCH(CH₃) |
| 626 | 5-(hydroxymethyl)isoxazolyl | OCH(CH₃) |
| 627 | 1,3-bis(1,1,1-trifluoroeth-2-yl)pyrazol-4-yl | OCH(CH₃) |
| 628 | 1-methyl-3-(trifluoromethyl)pyrazol-5-yl | OCH(CH₃) |
| 629 | 3-methylimidazol-2-yl | OCH(CH₃) |
| 630 | 4-methylimididazol-5-yl | OCH(CH₃) |
| 631 | 4-amino-2-methylpyrimidin-2-yl | OCH(CH₃) |
| 632 | 6-hydroxy-2-methylpyrimidin-4-yl | OCH(CH₃) |
| 633 | 3-hydroxynapth-2-yl | OCH(CH₃) |
| 634 | 2-benzothienyl | OCH(CH₃) |
| 635 | 3-chloro-2-benzothienyl | OCH(CH₃) |
| 636 | 1-naphthyl | OCH(CH₃) |
| 637 | 2-naphthyl | OCH(CH₃) |
| 638 | 3-indolyl | OCH(CH₃) |
| 639 | 2-benzimidazolyl | OCH(CH₃) |
| 640 | 6,7-dimethoxy-3-methyl-1,2,3,4- | OCH(CH₃) |
| | tetrahydroisochinolin-3-yl | |
| 641 | 2-thienyl | SCH₂ |
| 642 | 3-bromothien-2-yl | SCH₂ |
| 643 | 4-bromothien-2-yl | SCH₂ |
| 644 | 5-bromothien-2-yl | SCH₂ |
| 645 | 4,5-dibromthien-2-yl | SCH₂ |
| 646 | 3-chlorothien-2-yl | SCH₂ |
| 647 | 4-chlorothien-2-yl | SCH₂ |
| 648 | 5-chlorothien-2-yl | SCH₂ |
| 649 | 3-chloro-4-methylthien-2-yl | SCH₂ |
| 650 | 3-methylthien-2-yl | SCH₂ |
| 651 | 4-methylthien-2-yl | SCH₂ |
| 652 | 5-methylthien-2-yl | SCH₂ |
| 653 | 3-methoxythien-2-yl | SCH₂ |
| 654 | 3-methoxy-5-(trifluoromethyl)thien-2-yl | SCH₂ |
| 655 | 3-ethoxy-5-(trifluoromethyl)thien-2-yl | SCH₂ |
| 656 | 2-bromo-4,5-dimethylthien-2-yl | SCH₂ |
| 657 | pyridin-2-yl | SCH₂ |
| 658 | 6-methylthiopyridin-2-yl | SCH₂ |
| 659 | 3-fluoropyridin-2-yl | SCH₂ |
| 660 | 3-methoxypyridin-2-yl | SCH₂ |
| 661 | 6-methylpyridin-2-yl | SCH₂ |
| 662 | 5-nitropyridin-2-yl | SCH₂ |
| 663 | 3-fluoropyridin-2-yl | SCH₂ |
| 664 | 6-bromopyridin-2-yl | SCH₂ |
| 665 | 6-chloropyridin-2-yl | SCH₂ |
| 666 | 4-chloropyridin-2-yl | SCH₂ |
| 667 | 3,6-dichloropyridin-2-yl | SCH₂ |
| 668 | 6-fluoropyridin-2-yl | SCH₂ |
| 669 | 5-(trifluoromethyl)pyridin-2-yl | SCH₂ |
| 670 | 4-(trifluoromethyl)pyridin-2-yl | SCH₂ |
| 671 | 6-(trifluoromethyl)pyridin-2-yl | SCH₂ |
| 672 | 6-hydroxypyridin-2-yl | SCH₂ |
| 673 | 6-methoxypyridin-2-yl | SCH₂ |
| 674 | 3-hydroxypyridin-2-yl | SCH₂ |
| 675 | 6-phenylpyridin-2-yl | SCH₂ |
| 676 | pyridin-3-yl | SCH₂ |
| 677 | 6-chloropyridin-3-yl | SCH₂ |
| 678 | 2,6-dichloropyridin-3-yl | SCH₂ |
| 679 | 5,6-dichloropyridin-3-yl | SCH₂ |
| 680 | 2,6-dichloro-4-methylpyridin-3-yl | SCH₂ |
| 681 | 6-fluoropyridin-3-yl | SCH₂ |
| 682 | 6-bromopyridin-3-yl | SCH₂ |
| 683 | 5-bromopyridin-3-yl | SCH₂ |
| 684 | 2-phenoxypyridin-3-yl | SCH₂ |
| 685 | 1-methyl-2-hydroxypyridin-3-yl | SCH₂ |
| 686 | pyridin-4-yl | SCH₂ |
| 687 | 2-chloropyridin-4-yl | SCH₂ |
| 688 | 2,6-dichloropyridin-4-yl | SCH₂ |
| 689 | phenyl | SCH₂ |
| 690 | 2-fluorophenyl | SCH₂ |
| 691 | 3-fluorophenyl | SCH₂ |
| 692 | 4-fluorophenyl | SCH₂ |
| 693 | 2,4-difluorophenyl | SCH₂ |
| 694 | 2,5-difluorophenyl | SCH₂ |
| 695 | 2-chlorophenyl | SCH₂ |
| 696 | 3-chlorophenyl | SCH₂ |
| 697 | 4-chlorophenyl | SCH₂ |
| 698 | 2,3-dichlorophenyl | SCH₂ |
| 699 | 2,4-dichlorophenyl | SCH₂ |
| 700 | 2,5-dichlorophenyl | SCH₂ |
| 701 | 2,6-dichlorophenyl | SCH₂ |
| 702 | 2-chloro-6-fluorophenyl | SCH₂ |
| 703 | 2-chloro-4-fluorophenyl | SCH₂ |
| 704 | 1,2,3,4,5-pentafluorophenyl | SCH₂ |
| 705 | 2-bromophenyl | SCH₂ |
| 706 | 3-bromophenyl | SCH₂ |
| 707 | 4-bromophenyl | SCH₂ |
| 708 | 5-bromo-2-chlorophenyl | SCH₂ |
| 709 | 4-chloro-2-methylphenyl | SCH₂ |
| 710 | 3-fluoro-4-methylphenyl | SCH₂ |
| 711 | 2-methylphenyl | SCH₂ |
| 712 | 3-methylphenyl | SCH₂ |
| 713 | 4-methylphenyl | SCH₂ |
| 714 | 2,6-dimethylphenyl | SCH₂ |
| 715 | 2-(trifluoromethyl)phenyl | SCH₂ |
| 716 | 3-(trifluoromethyl)phenyl | SCH₂ |
| 717 | 4-(trifluoromethyl)phenyl | SCH₂ |
| 718 | 2-fluoro-4-(trifluoromethyl)phenyl | SCH₂ |
| 719 | 3,5-bis(trifluoromethyl)phenyl | SCH₂ |
| 720 | 4-tert.-butyl-phenyl | SCH₂ |
| 721 | 2-methoxyphenyl | SCH₂ |
| 722 | 3-methoxyphenyl | SCH₂ |
| 723 | 4-methoxyphenyl | SCH₂ |
| 724 | 3,4-dimethoxyphenyl | SCH₂ |
| 725 | 3,4,5-trimethoxyphenyl | SCH₂ |
| 726 | 2-ethoxyphenyl | SCH₂ |
| 727 | 3-ethoxyphenyl | SCH₂ |
| 728 | 4-ethoxyphenyl | SCH₂ |
| 729 | 2-methoxy-3-(methylthio)phenyl | SCH₂ |
| 730 | 2-nitrophenyl | SCH₂ |
| 731 | 3-nitrophenyl | SCH₂ |
| 732 | 4-nitrophenyl | SCH₂ |
| 733 | 2-hydroxyphenyl | SCH₂ |
| 734 | 3-hydroxyphenyl | SCH₂ |
| 735 | 4-hydroxyphenyl | SCH₂ |
| 736 | 4-hydroxy-3-methoxyphenyl | SCH₂ |
| 737 | 2-fluoro-4-hydroxyphenyl | SCH₂ |
| 738 | 2-aminophenyl | SCH₂ |
| 739 | 3-aminophenyl | SCH₂ |
| 740 | 4-aminophenyl | SCH₂ |
| 741 | 6-amino-2-fluorophenyl | SCH₂ |
| 742 | 4-(dimethylamino)phenyl | SCH₂ |
| 743 | 4-phenylphenyl | SCH₂ |
| 744 | 4-(methylsulfonyl)-2-nitrophenyl | SCH₂ |
| 745 | 5-methylpyrazin-2-yl | SCH₂ |
| 746 | 3-aminopyrazin-2-yl | SCH₂ |
| 747 | 2-furyl | SCH₂ |
| 748 | 5-bromofuran-2-yl | SCH₂ |
| 749 | 2-methylfuran-3-yl | SCH₂ |
| 750 | 5-isoxazolyl | SCH₂ |
| 751 | 3-methylisoxazolyl | SCH₂ |
| 752 | 3-(1-methylethyl)isoxazolyl | SCH₂ |
| 753 | 5-(chloromethyl)isoxazolyl | SCH₂ |
| 754 | 5-(hydroxymethyl)isoxazolyl | SCH₂ |
| 755 | 1,3-bis(1,1,1-trifluoroeth-2-yl)pyrazol-4-yl | SCH₂ |
| 756 | 1-methyl-3-(trifluoromethyl)pyrazol-5-yl | SCH₂ |
| 757 | 3-methylimidazol-2-yl | SCH₂ |
| 758 | 4-methylimididazol-5-yl | SCH₂ |
| 759 | 4-amino-2-methylpyrimidin-2-yl | SCH₂ |
| 760 | 6-hydroxy-2-methylpyrimidin-4-yl | SCH₂ |
| 761 | 3-hydroxynapth-2-yl | SCH₂ |
| 762 | 2-benzothienyl | SCH₂ |
| 763 | 3-chloro-2-benzothienyl | SCH₂ |
| 764 | 1-naphthyl | SCH₂ |
| 765 | 2-naphthyl | SCH₂ |
| 766 | 3-indolyl | SCH₂ |
| 767 | 2-benzimidazolyl | SCH₂ |
| 768 | 6,7-dimethoxy-3-methyl-1,2,3,4- | SCH₂ |
| | tetrahydroisochinolin-3-yl | |
| 769 | 2-thienyl | OCH₂CH₂CH₂ |
| 770 | 3-bromothien-2-yl | OCH₂CH₂CH₂ |
| 771 | 4-bromothien-2-yl | OCH₂CH₂CH₂ |
| 772 | 5-bromothien-2-yl | OCH₂CH₂CH₂ |
| 773 | 4,5-dibromthien-2-yl | OCH₂CH₂CH₂ |
| 774 | 3-chlorothien-2-yl | OCH₂CH₂CH₂ |
| 775 | 4-chlorothien-2-yl | OCH₂CH₂CH₂ |
| 776 | 5-chlorothien-2-yl | OCH₂CH₂CH₂ |
| 777 | 3-chloro-4-methylthien-2-yl | OCH₂CH₂CH₂ |
| 778 | 3-methylthien-2-yl | OCH₂CH₂CH₂ |
| 779 | 4-methylthien-2-yl | OCH₂CH₂CH₂ |
| 780 | 5-methylthien-2-yl | OCH₂CH₂CH₂ |
| 781 | 3-methoxythien-2-yl | OCH₂CH₂CH₂ |
| 782 | 3-methoxy-5-(trifluoromethyl)thien-2-yl | OCH₂CH₂CH₂ |
| 783 | 3-ethoxy-5-(trifluoromethyl)thien-2-yl | OCH₂CH₂CH₂ |
| 784 | 2-bromo-4,5-dimethylthien-2-yl | OCH₂CH₂CH₂ |
| 785 | pyridin-2-yl | OCH₂CH₂CH₂ |
| 786 | 6-methylthiopyridin-2-yl | OCH₂CH₂CH₂ |
| 787 | 3-fluoropyridin-2-yl | OCH₂CH₂CH₂ |
| 788 | 3-methoxypyridin-2-yl | OCH₂CH₂CH₂ |
| 789 | 6-methylpyridin-2-yl | OCH₂CH₂CH₂ |
| 790 | 5-nitropyridin-2-yl | OCH₂CH₂CH₂ |
| 791 | 3-fluoropyridin-2-yl | OCH₂CH₂CH₂ |
| 792 | 6-bromopyridin-2-yl | OCH₂CH₂CH₂ |
| 793 | 6-chloropyridin-2-yl | OCH₂CH₂CH₂ |
| 794 | 4-chloropyridin-2-yl | OCH₂CH₂CH₂ |
| 795 | 3,6-dichloropyridin-2-yl | OCH₂CH₂CH₂ |
| 796 | 6-fluoropyridin-2-yl | OCH₂CH₂CH₂ |
| 797 | 5-(trifluoromethyl)pyridin-2-yl | OCH₂CH₂CH₂ |
| 798 | 4-(trifluoromethyl)pyridin-2-yl | OCH₂CH₂CH₂ |
| 799 | 6-(trifluoromethyl)pyridin-2-yl | OCH₂CH₂CH₂ |
| 800 | 6-hydroxypyridin-2-yl | OCH₂CH₂CH₂ |
| 801 | 6-methoxypyridin-2-yl | OCH₂CH₂CH₂ |
| 802 | 3-hydroxypyridin-2-yl | OCH₂CH₂CH₂ |
| 803 | 6-phenylpyridin-2-yl | OCH₂CH₂CH₂ |
| 804 | pyridin-3-yl | OCH₂CH₂CH₂ |
| 805 | 6-chloropyridin-3-yl | OCH₂CH₂CH₂ |
| 806 | 2,6-dichloropyridin-3-yl | OCH₂CH₂CH₂ |
| 807 | 5,6-dichloropyridin-3-yl | OCH₂CH₂CH₂ |
| 808 | 2,6-dichloro-4-methylpyridin-3-yl | OCH₂CH₂CH₂ |
| 809 | 6-fluoropyridin-3-yl | OCH₂CH₂CH₂ |
| 810 | 6-bromopyridin-3-yl | OCH₂CH₂CH₂ |
| 811 | 5-bromopyridin-3-yl | OCH₂CH₂CH₂ |
| 812 | 2-phenoxypyridin-3-yl | OCH₂CH₂CH₂ |
| 813 | 1-methyl-2-hydroxypyridin-3-yl | OCH₂CH₂CH₂ |
| 814 | pyridin-4-yl | OCH₂CH₂CH₂ |
| 815 | 2-chloropyridin-4-yl | OCH₂CH₂CH₂ |
| 816 | 2,6-dichloropyridin-4-yl | OCH₂CH₂CH₂ |
| 817 | phenyl | OCH₂CH₂CH₂ |
| 818 | 2-fluorophenyl | OCH₂CH₂CH₂ |
| 819 | 3-fluorophenyl | OCH₂CH₂CH₂ |
| 820 | 4-fluorophenyl | OCH₂CH₂CH₂ |
| 821 | 2,4-difluorophenyl | OCH₂CH₂CH₂ |
| 822 | 2,5-difluorophenyl | OCH₂CH₂CH₂ |
| 823 | 2-chlorophenyl | OCH₂CH₂CH₂ |
| 824 | 3-chlorophenyl | OCH₂CH₂CH₂ |
| 825 | 4-chlorophenyl | OCH₂CH₂CH₂ |
| 826 | 2,3-dichlorophenyl | OCH₂CH₂CH₂ |
| 827 | 2,4-dichlorophenyl | OCH₂CH₂CH₂ |
| 828 | 2,5-dichlorophenyl | OCH₂CH₂CH₂ |
| 829 | 2,6-dichlorophenyl | OCH₂CH₂CH₂ |
| 830 | 2-chloro-6-fluorophenyl | OCH₂CH₂CH₂ |
| 831 | 2-chloro-4-fluorophenyl | OCH₂CH₂CH₂ |
| 832 | 1,2,3,4,5-pentafluorophenyl | OCH₂CH₂CH₂ |
| 833 | 2-bromophenyl | OCH₂CH₂CH₂ |
| 834 | 3-bromophenyl | OCH₂CH₂CH₂ |
| 835 | 4-bromophenyl | OCH₂CH₂CH₂ |
| 836 | 5-bromo-2-chlorophenyl | OCH₂CH₂CH₂ |
| 837 | 4-chloro-2-methylphenyl | OCH₂CH₂CH₂ |
| 838 | 3-fluoro-4-methylphenyl | OCH₂CH₂CH₂ |
| 839 | 2-methylphenyl | OCH₂CH₂CH₂ |
| 840 | 3-methylphenyl | OCH₂CH₂CH₂ |
| 841 | 4-methylphenyl | OCH₂CH₂CH₂ |
| 842 | 2,6-dimethylphenyl | OCH₂CH₂CH₂ |
| 843 | 2-(trifluoromethyl)phenyl | OCH₂CH₂CH₂ |
| 844 | 3-(trifluoromethyl)phenyl | OCH₂CH₂CH₂ |
| 845 | 4-(trifluoromethyl)phenyl | OCH₂CH₂CH₂ |
| 846 | 2-fluoro-4-(trifluoromethyl)phenyl | OCH₂CH₂CH₂ |
| 847 | 3,5-bis(trifluoromethyl)phenyl | OCH₂CH₂CH₂ |
| 848 | 4-tert.-butyl-phenyl | OCH₂CH₂CH₂ |
| 849 | 2-methoxyphenyl | OCH₂CH₂CH₂ |
| 850 | 3-methoxyphenyl | OCH₂CH₂CH₂ |
| 851 | 4-methoxyphenyl | OCH₂CH₂CH₂ |
| 852 | 3,4-dimethoxyphenyl | OCH₂CH₂CH₂ |
| 853 | 3,4,5-trimethoxyphenyl | OCH₂CH₂CH₂ |
| 854 | 2-ethoxyphenyl | OCH₂CH₂CH₂ |
| 855 | 3-ethoxyphenyl | OCH₂CH₂CH₂ |
| 856 | 4-ethoxyphenyl | OCH₂CH₂CH₂ |
| 857 | 2-methoxy-3-(methylthio)phenyl | OCH₂CH₂CH₂ |
| 858 | 2-nitrophenyl | OCH₂CH₂CH₂ |
| 859 | 3-nitrophenyl | OCH₂CH₂CH₂ |
| 860 | 4-nitrophenyl | OCH₂CH₂CH₂ |
| 861 | 2-hydroxyphenyl | OCH₂CH₂CH₂ |
| 862 | 3-hydroxyphenyl | OCH₂CH₂CH₂ |
| 863 | 4-hydroxyphenyl | OCH₂CH₂CH₂ |
| 864 | 4-hydroxy-3-methoxyphenyl | OCH₂CH₂CH₂ |
| 865 | 2-fluoro-4-hydroxyphenyl | OCH₂CH₂CH₂ |
| 866 | 2-aminophenyl | OCH₂CH₂CH₂ |
| 867 | 3-aminophenyl | OCH₂CH₂CH₂ |
| 868 | 4-aminophenyl | OCH₂CH₂CH₂ |
| 869 | 6-amino-2-fluorophenyl | OCH₂CH₂CH₂ |
| 870 | 4-(dimethylamino)phenyl | OCH₂CH₂CH₂ |
| 871 | 4-phenylphenyl | OCH₂CH₂CH₂ |
| 872 | 4-(methylsulfonyl)-2-nitrophenyl | OCH₂CH₂CH₂ |
| 873 | 5-methylpyrazin-2-yl | OCH₂CH₂CH₂ |
| 874 | 3-aminopyrazin-2-yl | OCH₂CH₂CH₂ |
| 875 | 2-furyl | OCH₂CH₂CH₂ |
| 876 | 5-bromofuran-2-yl | OCH₂CH₂CH₂ |
| 877 | 2-methylfuran-3-yl | OCH₂CH₂CH₂ |
| 878 | 5-isoxazolyl | OCH₂CH₂CH₂ |
| 879 | 3-methylisoxazolyl | OCH₂CH₂CH₂ |
| 880 | 3-(1-methylethyl)isoxazolyl | OCH₂CH₂CH₂ |
| 881 | 5-(chloromethyl)isoxazolyl | OCH₂CH₂CH₂ |
| 882 | 5-(hydroxymethyl)isoxazolyl | OCH₂CH₂CH₂ |
| 883 | 1,3-bis(1,1,1-trifluoroeth-2-yl)pyrazol-4-yl | OCH₂CH₂CH₂ |
| 884 | 1-methyl-3-(trifluoromethyl)pyrazol-5-yl | OCH₂CH₂CH₂ |
| 885 | 3-methylimidazol-2-yl | OCH₂CH₂CH₂ |
| 886 | 4-methylimididazol-5-yl | OCH₂CH₂CH₂ |
| 887 | 4-amino-2-methylpyrimidin-2-yl | OCH₂CH₂CH₂ |
| 888 | 6-hydroxy-2-methylpyrimidin-4-yl | OCH₂CH₂CH₂ |
| 889 | 3-hydroxynapth-2-yl | OCH₂CH₂CH₂ |
| 890 | 2-benzothienyl | OCH₂CH₂CH₂ |
| 891 | 3-chloro-2-benzothienyl | OCH₂CH₂CH₂ |
| 892 | 1-naphthyl | OCH₂CH₂CH₂ |
| 893 | 2-naphthyl | OCH₂CH₂CH₂ |
| 894 | 3-indolyl | OCH₂CH₂CH₂ |
| 895 | 2-benzimidazolyl | OCH₂CH₂CH₂ |
| 896 | 6,7-dimethoxy-3-methyl-1,2,3,4- | OCH₂CH₂CH₂ |
| | tetrahydroisochinolin-3-yl | |
| 897 | 2-thienyl | NHCH₂CH₂ |
| 898 | 3-bromothien-2-yl | NHCH₂CH₂ |
| 899 | 4-bromothien-2-yl | NHCH₂CH₂ |
| 900 | 5-bromothien-2-yl | NHCH₂CH₂ |
| 901 | 4,5-dibromthien-2-yl | NHCH₂CH₂ |
| 902 | 3-chlorothien-2-yl | NHCH₂CH₂ |
| 903 | 4-chlorothien-2-yl | NHCH₂CH₂ |
| 904 | 5-chlorothien-2-yl | NHCH₂CH₂ |
| 905 | 3-chloro-4-methylthien-2-yl | NHCH₂CH₂ |
| 906 | 3-methylthien-2-yl | NHCH₂CH₂ |
| 907 | 4-methylthien-2-yl | NHCH₂CH₂ |
| 908 | 5-methylthien-2-yl | NHCH₂CH₂ |
| 909 | 3-methoxythien-2-yl | NHCH₂CH₂ |
| 910 | 3-methoxy-5-(trifluoromethyl)thien-2-yl | NHCH₂CH₂ |
| 911 | 3-ethoxy-5-(trifluoromethyl)thien-2-yl | NHCH₂CH₂ |
| 912 | 2-bromo-4,5-dimethylthien-2-yl | NHCH₂CH₂ |
| 913 | pyridin-2-yl | NHCH₂CH₂ |
| 914 | 6-methylthiopyridin-2-yl | NHCH₂CH₂ |
| 915 | 3-fluoropyridin-2-yl | NHCH₂CH₂ |
| 916 | 3-methoxypyridin-2-yl | NHCH₂CH₂ |
| 917 | 6-methylpyridin-2-yl | NHCH₂CH₂ |
| 918 | 5-nitropyridin-2-yl | NHCH₂CH₂ |
| 919 | 3-fluoropyridin-2-yl | NHCH₂CH₂ |
| 920 | 6-bromopyridin-2-yl | NHCH₂CH₂ |
| 921 | 6-chloropyridin-2-yl | NHCH₂CH₂ |
| 922 | 4-chloropyridin-2-yl | NHCH₂CH₂ |
| 923 | 3,6-dichloropyridin-2-yl | NHCH₂CH₂ |
| 924 | 6-fluoropyridin-2-yl | NHCH₂CH₂ |
| 925 | 5-(trifluoromethyl)pyridin-2-yl | NHCH₂CH₂ |
| 926 | 4-(trifluoromethyl)pyridin-2-yl | NHCH₂CH₂ |
| 927 | 6-(trifluoromethyl)pyridin-2-yl | NHCH₂CH₂ |
| 928 | 6-hydroxypyridin-2-yl | NHCH₂CH₂ |
| 929 | 6-methoxypyridin-2-yl | NHCH₂CH₂ |
| 930 | 3-hydroxypyridin-2-yl | NHCH₂CH₂ |
| 931 | 6-phenylpyridin-2-yl | NHCH₂CH₂ |
| 932 | pyridin-3-yl | NHCH₂CH₂ |
| 933 | 6-chloropyridin-3-yl | NHCH₂CH₂ |
| 934 | 2,6-dichloropyridin-3-yl | NHCH₂CH₂ |
| 935 | 5,6-dichloropyridin-3-yl | NHCH₂CH₂ |
| 936 | 2,6-dichloro-4-methylpyridin-3-yl | NHCH₂CH₂ |
| 937 | 6-fluoropyridin-3-yl | NHCH₂CH₂ |
| 938 | 6-bromopyridin-3-yl | NHCH₂CH₂ |
| 939 | 5-bromopyridin-3-yl | NHCH₂CH₂ |
| 940 | 2-phenoxypyridin-3-yl | NHCH₂CH₂ |
| 941 | 1-methyl-2-hydroxypyridin-3-yl | NHCH₂CH₂ |
| 942 | pyridin-4-yl | NHCH₂CH₂ |
| 943 | 2-chloropyridin-4-yl | NHCH₂CH₂ |
| 944 | 2,6-dichloropyridin-4-yl | NHCH₂CH₂ |
| 945 | phenyl | NHCH₂CH₂ |
| 946 | 2-fluorophenyl | NHCH₂CH₂ |
| 947 | 3-fluorophenyl | NHCH₂CH₂ |
| 948 | 4-fluorophenyl | NHCH₂CH₂ |
| 949 | 2,4-difluorophenyl | NHCH₂CH₂ |
| 950 | 2,5-difluorophenyl | NHCH₂CH₂ |
| 951 | 2-chlorophenyl | NHCH₂CH₂ |
| 952 | 3-chlorophenyl | NHCH₂CH₂ |
| 953 | 4-chlorophenyl | NHCH₂CH₂ |
| 954 | 2,3-dichlorophenyl | NHCH₂CH₂ |
| 955 | 2,4-dichlorophenyl | NHCH₂CH₂ |
| 956 | 2,5-dichlorophenyl | NHCH₂CH₂ |
| 957 | 2,6-dichlorophenyl | NHCH₂CH₂ |
| 958 | 2-chloro-6-fluorophenyl | NHCH₂CH₂ |
| 959 | 2-chloro-4-fluorophenyl | NHCH₂CH₂ |
| 960 | 1,2,3,4,5-pentafluorophenyl | NHCH₂CH₂ |
| 961 | 2-bromophenyl | NHCH₂CH₂ |
| 962 | 3-bromophenyl | NHCH₂CH₂ |
| 963 | 4-bromophenyl | NHCH₂CH₂ |
| 964 | 5-bromo-2-chlorophenyl | NHCH₂CH₂ |
| 965 | 4-chloro-2-methylphenyl | NHCH₂CH₂ |
| 966 | 3-fluoro-4-methylphenyl | NHCH₂CH₂ |
| 967 | 2-methylphenyl | NHCH₂CH₂ |
| 968 | 3-methylphenyl | NHCH₂CH₂ |
| 969 | 4-methylphenyl | NHCH₂CH₂ |
| 970 | 2,6-dimethylphenyl | NHCH₂CH₂ |
| 971 | 2-(trifluoromethyl)phenyl | NHCH₂CH₂ |
| 972 | 3-(trifluoromethyl)phenyl | NHCH₂CH₂ |
| 973 | 4-(trifluoromethyl)phenyl | NHCH₂CH₂ |
| 974 | 2-fluoro-4-(trifluoromethyl)phenyl | NHCH₂CH₂ |
| 975 | 3,5-bis(trifluoromethyl)phenyl | NHCH₂CH₂ |
| 976 | 4-tert.-butyl-phenyl | NHCH₂CH₂ |
| 977 | 2-methoxyphenyl | NHCH₂CH₂ |
| 978 | 3-methoxyphenyl | NHCH₂CH₂ |
| 979 | 4-methoxyphenyl | NHCH₂CH₂ |
| 980 | 3,4-dimethoxyphenyl | NHCH₂CH₂ |
| 981 | 3,4,5-trimethoxyphenyl | NHCH₂CH₂ |
| 982 | 2-ethoxyphenyl | NHCH₂CH₂ |
| 983 | 3-ethoxyphenyl | NHCH₂CH₂ |
| 984 | 4-ethoxyphenyl | NHCH₂CH₂ |
| 985 | 2-methoxy-3-(methylthio)phenyl | NHCH₂CH₂ |
| 986 | 2-nitrophenyl | NHCH₂CH₂ |
| 987 | 3-nitrophenyl | NHCH₂CH₂ |
| 988 | 4-nitrophenyl | NHCH₂CH₂ |
| 989 | 2-hydroxyphenyl | NHCH₂CH₂ |
| 990 | 3-hydroxyphenyl | NHCH₂CH₂ |
| 991 | 4-hydroxyphenyl | NHCH₂CH₂ |
| 992 | 4-hydroxy-3-methoxyphenyl | NHCH₂CH₂ |
| 993 | 2-fluoro-4-hydroxyphenyl | NHCH₂CH₂ |
| 994 | 2-aminophenyl | NHCH₂CH₂ |
| 995 | 3-aminophenyl | NHCH₂CH₂ |
| 996 | 4-aminophenyl | NHCH₂CH₂ |
| 997 | 6-amino-2-fluorophenyl | NHCH₂CH₂ |
| 998 | 4-(dimethylamino)phenyl | NHCH₂CH₂ |
| 999 | 4-phenylphenyl | NHCH₂CH₂ |
| 1000 | 4-(methylsulfonyl)-2-nitrophenyl | NHCH₂CH₂ |
| 1001 | 5-methylpyrazin-2-yl | NHCH₂CH₂ |
| 1002 | 3-aminopyrazin-2-yl | NHCH₂CH₂ |
| 1003 | 2-furyl | NHCH₂CH₂ |
| 1004 | 5-bromofuran-2-yl | NHCH₂CH₂ |
| 1005 | 2-methylfuran-3-yl | NHCH₂CH₂ |
| 1006 | 5-isoxazolyl | NHCH₂CH₂ |
| 1007 | 3-methylisoxazolyl | NHCH₂CH₂ |
| 1008 | 3-(1-methylethyl)isoxazolyl | NHCH₂CH₂ |
| 1009 | 5-(chloromethyl)isoxazolyl | NHCH₂CH₂ |
| 1010 | 5-(hydroxymethyl)isoxazolyl | NHCH₂CH₂ |
| 1011 | 1,3-bis(1,1,1-trifluoroeth-2-yl)pyrazol-4-yl | NHCH₂CH₂ |
| 1012 | 1-methyl-3-(trifluoromethy)pyrazol-5-yl | NHCH₂CH₂ |
| 1013 | 3-methylimidazol-2-yl | NHCH₂CH₂ |
| 1014 | 4-methylimididazol-5-yl | NHCH₂CH₂ |
| 1015 | 4-amino-2-methylpyrimidin-2-yl | NHCH₂CH₂ |
| 1016 | 6-hydroxy-2-methylpyrimidin-4-yl | NHCH₂CH₂ |
| 1017 | 3-hydroxynapth-2-yl | NHCH₂CH₂ |
| 1018 | 2-benzothienyl | NHCH₂CH₂ |
| 1019 | 3-chloro-2-benzothienyl | NHCH₂CH₂ |
| 1020 | 1-naphthyl | NHCH₂CH₂ |
| 1021 | 2-naphthyl | NHCH₂CH₂ |
| 1022 | 3-indolyl | NHCH₂CH₂ |
| 1023 | 2-benzimidazolyl | NHCH₂CH₂ |
| 1024 | 6,7-dimethoxy-3-methyl-1,2,3,4- | NHCH₂CH₂ |
| | tetrahydroisochinolin-3-yl | |
| 1025 | 2-thienyl | NHC(O)CH₂ |
| 1026 | 3-bromothien-2-yl | NHC(O)CH₂ |
| 1027 | 4-bromothien-2-yl | NHC(O)CH₂ |
| 1028 | 5-bromothien-2-yl | NHC(O)CH₂ |
| 1029 | 4,5-dibromthien-2-yl | NHC(O)CH₂ |
| 1030 | 3-chlorothien-2-yl | NHC(O)CH₂ |
| 1031 | 4-chlorothien-2-yl | NHC(O)CH₂ |
| 1032 | 5-chlorothien-2-yl | NHC(O)CH₂ |
| 1033 | 3-chloro-4-methylthien-2-yl | NHC(O)CH₂ |
| 1034 | 3-methylthien-2-yl | NHC(O)CH₂ |
| 1035 | 4-methylthien-2-yl | NHC(O)CH₂ |
| 1036 | 5-methylthien-2-yl | NHC(O)CH₂ |
| 1037 | 3-methoxythien-2-yl | NHC(O)CH₂ |
| 1038 | 3-methoxy-5-(trifluoromethyl)thien-2-yl | NHC(O)CH₂ |
| 1039 | 3-ethoxy-5-(trifluoromethyl)thien-2-yl | NHC(O)CH₂ |
| 1040 | 2-bromo-4,5-dimethylthien-2-yl | NHC(O)CH₂ |
| 1041 | pyridin-2-yl | NHC(O)CH₂ |
| 1042 | 6-methylthiopyridin-2-yl | NHC(O)CH₂ |
| 1043 | 3-fluoropyridin-2-yl | NHC(O)CH₂ |
| 1044 | 3-methoxypyridin-2-yl | NHC(O)CH₂ |
| 1045 | 6-methylpyridin-2-yl | NHC(O)CH₂ |
| 1046 | 5-nitropyridin-2-yl | NHC(O)CH₂ |
| 1047 | 3-fluoropyridin-2-yl | NHC(O)CH₂ |
| 1048 | 6-bromopyridin-2-yl | NHC(O)CH₂ |
| 1049 | 6-chloropyridin-2-yl | NHC(O)CH₂ |
| 1050 | 4-chloropyridin-2-yl | NHC(O)CH₂ |
| 1051 | 3,6-dichloropyridin-2-yl | NHC(O)CH₂ |
| 1052 | 6-fluoropyridin-2-yl | NHC(O)CH₂ |
| 1053 | 5-(trifluoromethyl)pyridin-2-yl | NHC(O)CH₂ |
| 1054 | 4-(trifluoromethyl)pyridin-2-yl | NHC(O)CH₂ |
| 1055 | 6-(trifluoromethyl)pyridin-2-yl | NHC(O)CH₂ |
| 1056 | 6-hydroxypyridin-2-yl | NHC(O)CH₂ |
| 1057 | 6-methoxypyridin-2-yl | NHC(O)CH₂ |
| 1058 | 3-hydroxypyridin-2-yl | NHC(O)CH₂ |
| 1059 | 6-phenylpyridin-2-yl | NHC(O)CH₂ |
| 1060 | pyridin-3-yl | NHC(O)CH₂ |
| 1061 | 6-chloropyridin-3-yl | NHC(O)CH₂ |
| 1062 | 2,6-dichloropyridin-3-yl | NHC(O)CH₂ |
| 1063 | 5,6-dichloropyridin-3-yl | NHC(O)CH₂ |
| 1064 | 2,6-dichloro-4-methylpyridin-3-yl | NHC(O)CH₂ |
| 1065 | 6-fluoropyridin-3-yl | NHC(O)CH₂ |
| 1066 | 6-bromopyridin-3-yl | NHC(O)CH₂ |
| 1067 | 5-bromopyridin-3-yl | NHC(O)CH₂ |
| 1068 | 2-phenoxypyridin-3-yl | NHC(O)CH₂ |
| 1069 | 1-methyl-2-hydroxypyridin-3-yl | NHC(O)CH₂ |
| 1070 | pyridin-4-yl | NHC(O)CH₂ |
| 1071 | 2-chloropyridin-4-yl | NHC(O)CH₂ |
| 1072 | 2,6-dichloropyridin-4-yl | NHC(O)CH₂ |
| 1073 | phenyl | NHC(O)CH₂ |
| 1074 | 2-fluorophenyl | NHC(O)CH₂ |
| 1075 | 3-fluorophenyl | NHC(O)CH₂ |
| 1076 | 4-fluorophenyl | NHC(O)CH₂ |
| 1077 | 2,4-difluorophenyl | NHC(O)CH₂ |
| 1078 | 2,5-difluorophenyl | NHC(O)CH₂ |
| 1079 | 2-chlorophenyl | NHC(O)CH₂ |
| 1080 | 3-chlorophenyl | NHC(O)CH₂ |
| 1081 | 4-chlorophenyl | NHC(O)CH₂ |
| 1082 | 2,3-dichlorophenyl | NHC(O)CH₂ |
| 1083 | 2,4-dichlorophenyl | NHC(O)CH₂ |
| 1084 | 2,5-dichlorophenyl | NHC(O)CH₂ |
| 1085 | 2,6-dichlorophenyl | NHC(O)CH₂ |
| 1086 | 2-chloro-6-fluorophenyl | NHC(O)CH₂ |
| 1087 | 2-chloro-4-fluorophenyl | NHC(O)CH₂ |
| 1088 | 1,2,3,4,5-pentafluorophenyl | NHC(O)CH₂ |
| 1089 | 2-bromophenyl | NHC(O)CH₂ |
| 1090 | 3-bromophenyl | NHC(O)CH₂ |
| 1091 | 4-bromophenyl | NHC(O)CH₂ |
| 1092 | 5-bromo-2-chlorophenyl | NHC(O)CH₂ |
| 1093 | 4-chloro-2-methylphenyl | NHC(O)CH₂ |
| 1094 | 3-fluoro-4-methylphenyl | NHC(O)CH₂ |
| 1095 | 2-methylphenyl | NHC(O)CH₂ |
| 1096 | 3-methylphenyl | NHC(O)CH₂ |
| 1097 | 4-methylphenyl | NHC(O)CH₂ |
| 1098 | 2,6-dimethylphenyl | NHC(O)CH₂ |
| 1099 | 2-(trifluoromethyl)phenyl | NHC(O)CH₂ |
| 1100 | 3-(trifluoromethyl)phenyl | NHC(O)CH₂ |
| 1101 | 4-(trifluoromethyl)phenyl | NHC(O)CH₂ |
| 1102 | 2-fluoro-4-(trifluoromethyl)phenyl | NHC(O)CH₂ |
| 1103 | 3,5-bis(trifluoromethyl)phenyl | NHC(O)CH₂ |
| 1104 | 4-tert.-butyl-phenyl | NHC(O)CH₂ |
| 1105 | 2-methoxyphenyl | NHC(O)CH₂ |
| 1106 | 3-methoxyphenyl | NHC(O)CH₂ |
| 1107 | 4-methoxyphenyl | NHC(O)CH₂ |
| 1108 | 3,4-dimethoxyphenyl | NHC(O)CH₂ |
| 1109 | 3,4,5-trimethoxyphenyl | NHC(O)CH₂ |
| 1110 | 2-ethoxyphenyl | NHC(O)CH₂ |
| 1111 | 3-ethoxyphenyl | NHC(O)CH₂ |
| 1112 | 4-ethoxyphenyl | NHC(O)CH₂ |
| 1113 | 2-methoxy-3-(methylthio)phenyl | NHC(O)CH₂ |
| 1114 | 2-nitrophenyl | NHC(O)CH₂ |
| 1115 | 3-nitrophenyl | NHC(O)CH₂ |
| 1116 | 4-nitrophenyl | NHC(O)CH₂ |
| 1117 | 2-hydroxyphenyl | NHC(O)CH₂ |
| 1118 | 3-hydroxyphenyl | NHC(O)CH₂ |
| 1119 | 4-hydroxyphenyl | NHC(O)CH₂ |
| 1120 | 4-hydroxy-3-methoxyphenyl | NHC(O)CH₂ |
| 1121 | 2-fluoro-4-hydroxyphenyl | NHC(O)CH₂ |
| 1122 | 2-aminophenyl | NHC(O)CH₂ |
| 1123 | 3-aminophenyl | NHC(O)CH₂ |
| 1124 | 4-aminophenyl | NHC(O)CH₂ |
| 1125 | 6-amino-2-fluorophenyl | NHC(O)CH₂ |
| 1126 | 4-(dimethylamino)phenyl | NHC(O)CH₂ |
| 1127 | 4-phenylphenyl | NHC(O)CH₂ |
| 1128 | 4-(methylsulfonyl)-2-nitrophenyl | NHC(O)CH₂ |
| 1129 | 5-methylpyrazin-2-yl | NHC(O)CH₂ |
| 1130 | 3-aminopyrazin-2-yl | NHC(O)CH₂ |
| 1131 | 2-furyl | NHC(O)CH₂ |
| 1132 | 5-bromofuran-2-yl | NHC(O)CH₂ |
| 1133 | 2-methylfuran-3-yl | NHC(O)CH₂ |
| 1134 | 5-isoxazolyl | NHC(O)CH₂ |
| 1135 | 3-methylisoxazolyl | NHC(O)CH₂ |
| 1136 | 3-(1-methylethyl)isoxazolyl | NHC(O)CH₂ |
| 1137 | 5-(chloromethyl)isoxazolyl | NHC(O)CH₂ |
| 1138 | 5-(hydroxymethyl)isoxazolyl | NHC(O)CH₂ |
| 1139 | 1,3-bis(1,1,1-trifluoroeth-2-yl)pyrazol-4-yl | NHC(O)CH₂ |
| 1140 | 1-methyl-3-(trifluoromethyl)pyrazol-5-yl | NHC(O)CH₂ |
| 1141 | 3-methylimidazol-2-yl | NHC(O)CH₂ |
| 1142 | 4-methylimididazol-5-yl | NHC(O)CH₂ |
| 1143 | 4-amino-2-methylpyrimidin-2-yl | NHC(O)CH₂ |
| 1144 | 6-hydroxy-2-methylpyrimidin-4-yl | NHC(O)CH₂ |
| 1145 | 3-hydroxynapth-2-yl | NHC(O)CH₂ |
| 1146 | 2-benzothienyl | NHC(O)CH₂ |
| 1147 | 3-chloro-2-benzothienyl | NHC(O)CH₂ |
| 1148 | 1-naphthyl | NHC(O)CH₂ |
| 1149 | 2-naphthyl | NHC(O)CH₂ |
| 1150 | 3-indolyl | NHC(O)CH₂ |
| 1151 | 2-benzimidazolyl | NHC(O)CH₂ |
| 1152 | 6,7-dimethoxy-3-methyl-1,2,3,4- | NHC(O)CH₂ |
| | tetrahydroisochinolin-3-yl | |
| 1153 | 2-thienyl | SCH(CH₃) |
| 1154 | 3-bromothien-2-yl | SCH(CH₃) |
| 1155 | 4-bromothien-2-yl | SCH(CH₃) |
| 1156 | 5-bromothien-2-yl | SCH(CH₃) |
| 1157 | 4,5-dibromthien-2-yl | SCH(CH₃) |
| 1158 | 3-chlorothien-2-yl | SCH(CH₃) |
| 1159 | 4-chlorothien-2-yl | SCH(CH₃) |
| 1160 | 5-chlorothien-2-yl | SCH(CH₃) |
| 1161 | 3-chloro-4-methylthien-2-yl | SCH(CH₃) |
| 1162 | 3-methylthien-2-yl | SCH(CH₃) |
| 1163 | 4-methylthien-2-yl | SCH(CH₃) |
| 1164 | 5-methylthien-2-yl | SCH(CH₃) |
| 1165 | 3-methoxythien-2-yl | SCH(CH₃) |
| 1166 | 3-methoxy-5-(trifluoromethyl)thien-2-yl | SCH(CH₃) |
| 1167 | 3-ethoxy-5-(trifluoromethyl)thien-2-yl | SCH(CH₃) |
| 1168 | 2-bromo-4,5-dimethylthien-2-yl | SCH(CH₃) |
| 1169 | pyridin-2-yl | SCH(CH₃) |
| 1170 | 6-methylthiopyridin-2-yl | SCH(CH₃) |
| 1171 | 3-fluoropyridin-2-yl | SCH(CH₃) |
| 1172 | 3-methoxypyridin-2-yl | SCH(CH₃) |
| 1173 | 6-methylpyridin-2-yl | SCH(CH₃) |
| 1174 | 5-nitropyridin-2-yl | SCH(CH₃) |
| 1175 | 3-fluoropyridin-2-yl | SCH(CH₃) |
| 1176 | 6-bromopyridin-2-yl | SCH(CH₃) |
| 1177 | 6-chloropyridin-2-yl | SCH(CH₃) |
| 1178 | 4-chloropyridin-2-yl | SCH(CH₃) |
| 1179 | 3,6-dichloropyridin-2-yl | SCH(CH₃) |
| 1180 | 6-fluoropyridin-2-yl | SCH(CH₃) |
| 1181 | 5-(trifluoromethyl)pyridin-2-yl | SCH(CH₃) |
| 1182 | 4-(trifluoromethyl)pyridin-2-yl | SCH(CH₃) |
| 1183 | 6-(trifluoromethyl)pyridin-2-yl | SCH(CH₃) |
| 1184 | 6-hydroxypyridin-2-yl | SCH(CH₃) |
| 1185 | 6-methoxypyridin-2-yl | SCH(CH₃) |
| 1186 | 3-hydroxypyridin-2-yl | SCH(CH₃) |
| 1187 | 6-phenylpyridin-2-yl | SCH(CH₃) |
| 1188 | pyridin-3-yl | SCH(CH₃) |
| 1189 | 6-chloropyridin-3-yl | SCH(CH₃) |
| 1190 | 2,6-dichloropyridin-3-yl | SCH(CH₃) |
| 1191 | 5,6-dichloropyridin-3-yl | SCH(CH₃) |
| 1192 | 2,6-dichloro-4-methylpyridin-3-yl | SCH(CH₃) |
| 1193 | 6-fluoropyridin-3-yl | SCH(CH₃) |
| 1194 | 6-bromopyridin-3-yl | SCH(CH₃) |
| 1195 | 5-bromopyridin-3-yl | SCH(CH₃) |
| 1196 | 2-phenoxypyridin-3-yl | SCH(CH₃) |
| 1197 | 1-methyl-2-hydroxypyridin-3-yl | SCH(CH₃) |
| 1198 | pyridin-4-yl | SCH(CH₃) |
| 1199 | 2-chloropyridin-4-yl | SCH(CH₃) |
| 1200 | 2,6-dichloropyridin-4-yl | SCH(CH₃) |
| 1201 | phenyl | SCH(CH₃) |
| 1202 | 2-fluorophenyl | SCH(CH₃) |
| 1203 | 3-fluorophenyl | SCH(CH₃) |
| 1204 | 4-fluorophenyl | SCH(CH₃) |
| 1205 | 2,4-difluorophenyl | SCH(CH₃) |
| 1206 | 2,5-difluorophenyl | SCH(CH₃) |
| 1207 | 2-chlorophenyl | SCH(CH₃) |
| 1208 | 3-chlorophenyl | SCH(CH₃) |
| 1209 | 4-chlorophenyl | SCH(CH₃) |
| 1210 | 2,3-dichlorophenyl | SCH(CH₃) |
| 1211 | 2,4-dichlorophenyl | SCH(CH₃) |
| 1212 | 2,5-dichlorophenyl | SCH(CH₃) |
| 1213 | 2,6-dichlorophenyl | SCH(CH₃) |
| 1214 | 2-chloro-6-fluorophenyl | SCH(CH₃) |
| 1215 | 2-chloro-4-fluorophenyl | SCH(CH₃) |
| 1216 | 1,2,3,4,5-pentafluorophenyl | SCH(CH₃) |
| 1217 | 2-bromophenyl | SCH(CH₃) |
| 1218 | 3-bromophenyl | SCH(CH₃) |
| 1219 | 4-bromophenyl | SCH(CH₃) |
| 1220 | 5-bromo-2-chlorophenyl | SCH(CH₃) |
| 1221 | 4-chloro-2-methylphenyl | SCH(CH₃) |
| 1222 | 3-fluoro-4-methylphenyl | SCH(CH₃) |
| 1223 | 2-methylphenyl | SCH(CH₃) |
| 1224 | 3-methylphenyl | SCH(CH₃) |
| 1225 | 4-methylphenyl | SCH(CH₃) |
| 1226 | 2,6-dimethylphenyl | SCH(CH₃) |
| 1227 | 2-(trifluoromethyl)phenyl | SCH(CH₃) |
| 1228 | 3-(trifluoromethyl)phenyl | SCH(CH₃) |
| 1229 | 4-(trifluoromethyl)phenyl | SCH(CH₃) |
| 1230 | 2-fluoro-4-(trifluoromethyl)phenyl | SCH(CH₃) |
| 1231 | 3,5-bis(trifluoromethyl)phenyl | SCH(CH₃) |
| 1232 | 4-tert.-butyl-phenyl | SCH(CH₃) |
| 1233 | 2-methoxyphenyl | SCH(CH₃) |
| 1234 | 3-methoxyphenyl | SCH(CH₃) |
| 1235 | 4-methoxyphenyl | SCH(CH₃) |
| 1236 | 3,4-dimethoxyphenyl | SCH(CH₃) |
| 1237 | 3,4,5-trimethoxyphenyl | SCH(CH₃) |
| 1238 | 2-ethoxyphenyl | SCH(CH₃) |
| 1239 | 3-ethoxyphenyl | SCH(CH₃) |
| 1240 | 4-ethoxyphenyl | SCH(CH₃) |
| 1241 | 2-methoxy-3-(methylthio)phenyl | SCH(CH₃) |
| 1242 | 2-nitrophenyl | SCH(CH₃) |
| 1243 | 3-nitrophenyl | SCH(CH₃) |
| 1244 | 4-nitrophenyl | SCH(CH₃) |
| 1245 | 2-hydroxyphenyl | SCH(CH₃) |
| 1246 | 3-hydroxyphenyl | SCH(CH₃) |
| 1247 | 4-hydroxyphenyl | SCH(CH₃) |
| 1248 | 4-hydroxy-3-methoxyphenyl | SCH(CH₃) |
| 1249 | 2-fluoro-4-hydroxyphenyl | SCH(CH₃) |
| 1250 | 2-aminophenyl | SCH(CH₃) |
| 1251 | 3-aminophenyl | SCH(CH₃) |
| 1252 | 4-aminophenyl | SCH(CH₃) |
| 1253 | 6-amino-2-fluorophenyl | SCH(CH₃) |
| 1254 | 4-(dimethylamino)phenyl | SCH(CH₃) |
| 1255 | 4-phenylphenyl | SCH(CH₃) |
| 1256 | 4-(methylsulfonyl)-2-nitrophenyl | SCH(CH₃) |
| 1257 | 5-methylpyrazin-2-yl | SCH(CH₃) |
| 1258 | 3-aminopyrazin-2-yl | SCH(CH₃) |
| 1259 | 2-furyl | SCH(CH₃) |
| 1260 | 5-bromofuran-2-yl | SCH(CH₃) |
| 1261 | 2-methylfuran-3-yl | SCH(CH₃) |
| 1262 | 5-isoxazolyl | SCH(CH₃) |
| 1263 | 3-methylisoxazolyl | SCH(CH₃) |
| 1264 | 3-(1-methylethyl)isoxazolyl | SCH(CH₃) |
| 1265 | 5-(chloromethyl)isoxazolyl | SCH(CH₃) |
| 1266 | 5-(hydroxymethyl)isoxazolyl | SCH(CH₃) |
| 1267 | 1,3-bis(1,1,1-trifluoroeth-2-yl)pyrazol-4-yl | SCH(CH₃) |
| 1268 | 1-methyl-3-(trifluoromethyl)pyrazol-5-yl | SCH(CH₃) |
| 1269 | 3-methylimidazol-2-yl | SCH(CH₃) |
| 1270 | 4-methylimididazol-5-yl | SCH(CH₃) |
| 1271 | 4-amino-2-methylpyrimidin-2-yl | SCH(CH₃) |
| 1272 | 6-hydroxy-2-methylpyrimidin-4-yl | SCH(CH₃) |
| 1273 | 3-hydroxynapth-2-yl | SCH(CH₃) |
| 1274 | 2-benzothienyl | SCH(CH₃) |
| 1275 | 3-chloro-2-benzothienyl | SCH(CH₃) |
| 1276 | 1-naphthyl | SCH(CH₃) |
| 1277 | 2-naphthyl | SCH(CH₃) |
| 1278 | 3-indolyl | SCH(CH₃) |
| 1279 | 2-benzimidazolyl | SCH(CH₃) |
| 1280 | 6,7-dimethoxy-3-methyl-1,2,3,4- | SCH(CH₃) |
| | tetrahydroisochinolin-3-yl | |

| | |
|---|---|
| 1281 | CH₃ |
| 1282 | CH₂CH₃ |
| 1283 | CH₂CH₂CH₃ |
| 1284 | CH₂CH₂CH₂CH₃ |
| 1285 | CH₂—(CH₂)₃—CH₃ |
| 1286 | CH₂—(CH₂)₄—CH₃ |
| 1287 | CH₂—(CH₂)₅—CH₃ |
| 1288 | CH₂—(CH₂)₆—CH₃ |
| 1289 | CH₂CO₂CH₃ |
| 1290 | CH₂CO₂C₂H₅ |
| 1291 | CH₂(CH₂)₃OH |
| 1292 | CH₂(CH₂)₄OH |
| 1293 | CH₂CH(OCH₃)₂ |
| 1294 | CH₂CH(OC₂H₅)₂ |

In table A the heteroatom of the moiety Q is attached to the radical A.

Another very preferred embodiment of the invention relates to compounds of the general formula Ia′:

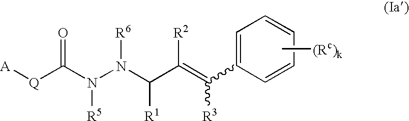

wherein k is 0, 1, 2 or 3, and wherein A, Q, R¹, R², R³, R⁵, R⁶ and R^{c} are as defined above. Amongst the compounds Ia′ those are preferred, wherein Q, A, R¹, R², R³, R⁵, R⁶ and R^{c} have the meanings given as preferred.

Examples of compounds Ia′ are given in the following tables 181 to 360:

Tables 181 to 270:

Compounds of the formula Ia′, wherein R¹ is H, R³ is H, R²is H, R⁵ is H, R⁶ is CH₃ and (R^{c})ₖ are as defined in one of the tables 1 to 90 and Q and A are given in table A.

Tables 271 to 360:

Compounds of the formula Ia′, wherein R¹ is H, R³is H, R²is H, R⁵ is CH₃, R⁶ is CH₃ and (R^{c})ₖ are as defined in one of the tables 1 to 90 and Q and A are given in table A.

Another very preferred embodiment of the invention relates to compounds of the general formula Ib:

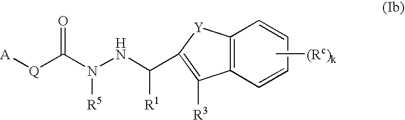

wherein k is 0, 1, 2 or 3, and wherein A, Q, R¹, R³, R⁵ and R^{c} are as defined above and Y is as defined above. Amongst the compounds Ia those are preferred, wherein Y is O, OCH₂ or S and wherein Q, A, R¹, R³, R⁵ and R^{c} have the meanings given as preferred.

Examples of compounds Ib are given in the following tables 361 to 378:

Table 361:

Compounds of the formula Ia, wherein Y is O, R¹ is H, R³ is H, R⁵ is H and k is 0 (i.e. (R^{c})ₖ is absent) and wherein Q and A are given in table A;

Table 362:

Compounds of the formula Ia, wherein Y is O, R¹ is H, R³ is H, R⁵ is CH₃ and k is 0 (i.e. (R^{c})ₖ is absent) and wherein Q and A are given in table A;

Table 363:

Compounds of the formula Ia, wherein Y is S, R¹ is H, R³ is H, R⁵ is H and k is 0 (i.e. (R^{c})ₖ is absent) and wherein Q and A are given in table A;

Table 364:

Compounds of the formula Ia, wherein Y is S, R¹ is H, R³ is H, R⁵ is CH₃ and k is 0 (i.e. (R^{c})ₖ is absent) and wherein Q and A are given in table A;

Table 365:

Compounds of the formula Ia, wherein Y is O, R¹ is H, R³ is H, R⁵ is H and (R^{c})ₖ is 4-Cl and wherein Q and A are given in table A;

Table 366:

Compounds of the formula Ia, wherein Y is S, R¹ is H, R³ is H, R⁵ is H and (R^{c})ₖ is 4-Cl and wherein Q and A are given in table A;

Table 367:

Compounds of the formula Ia, wherein Y is O, R¹ is H, R³ is H, R⁵ is CH₃ and (R^{c})ₖ is 4-Cl and wherein Q and A are given in table A;

Table 368:

Compounds of the formula Ia, wherein Y is S, R¹ is H, R³ is H, R⁵ is CH₃ and (R^{c})ₖ is 4-Cl and wherein Q and A are given in table A;

Table 369:

Compounds of the formula Ia, wherein Y is O, R¹ is H, R³ is H, R⁵ is H and (R^{c})ₖ is 4-Cl, 5-NO₂ and wherein Q and A are given in table A;

Table 370:

Compounds of the formula Ia, wherein Y is S, R¹ is H, R³ is H, R⁵ is H and (R^{c})ₖ is 4-Cl, 5-NO₂ and wherein Q and A are given in table A;

Table 371:

Compounds of the formula Ia, wherein Y is O, R¹ is H, R³ is H, R⁵ is CH₃ and (R^{c})ₖ is 4-Cl, 5-NO₂ and wherein Q and A are given in table A;

Table 372:

Compounds of the formula Ia, wherein Y is S, R¹ is H, R³ is H, R⁵ is CH₃ and (R^{c})ₖ is 4-Cl, 5-NO₂ and wherein Q and A are given in table A;

Table 373:

Compounds of the formula Ia, wherein Y is OCH₂ (the oxygen atom is attached to the phenyl ring), R¹ is H, R³ is H, R⁵ is H and k is 0 (i.e. (R^{c})ₖ is absent) and wherein Q and A are given in table A;

Table 374:

Compounds of the formula Ia, wherein Y is OCH₂ (the oxygen atom is attached to the phenyl ring), R¹ is H, R³ is H, R⁵ is CH₃ and k is 0 (i.e. (R^{c})ₖ is absent) and wherein Q and A are given in table A;

Table 375:

Compounds of the formula Ia, wherein Y is OCH₂ (the oxygen atom is attached to the phenyl ring), R¹ is H, R³ is H, R⁵ is H and (R^{c})ₖ is 4-Cl and wherein Q and A are given in table A;

Table 376:

Compounds of the formula Ia, wherein Y is OCH₂ (the oxygen atom is attached to the phenyl ring), R¹ is H, R³ is H, R⁶ is CH₃ and (R^{c})ₖ is 4-Cl and wherein Q and A are given in table A;

Table 377:

Compounds of the formula Ia, wherein Y is OCH₂ (the oxygen atom is attached to the phenyl ring), R¹ is H, R³ is H, R⁵ is H and (R^{c})ₖ is 4-Cl, 5-NO₂ and wherein Q and A are given in table A;

Table 378:

Compounds of the formula Ia, wherein Y is OCH₂ (the oxygen atom is attached to the phenyl ring), R¹ is H, R³ is H, R⁵ is CH₃ and (R_{c})ₖ is 4-Cl, 5-NO₂ and wherein Q and A are given in table A.

The compounds of the formula I may be readily synthesized using techniques generally known by synthetic organic chemists.

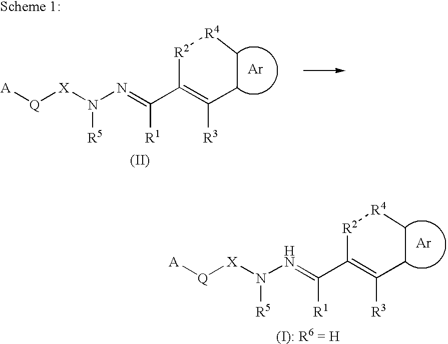

Compounds of formula I, wherein R⁶ is H are obtainable from compounds II by hydrogenation of the C═N-double bond in II according to standard methods of organic chemistry well known in the art and described e.g. in US 2002022730, Tetrahedron 1994, 50, 4399-4428, Tetrahedron 2002, 58, 7925-7932 or Trends in Heterocyclic Chemistry 1997, Vol 5, 17-36.

Hydrazides I carrying substituents R⁶ different from hydrogen are obtainable by functionalyzing hydrazides I with R⁶═H by well known methods such as described in Letters in Peptide Science 2003, 10, 1-9, J. Am. Chem. Soc. 2004, 126, 5366-5367 or Synthetic Communications 2002, 32, 3603-3610 (see scheme 2).

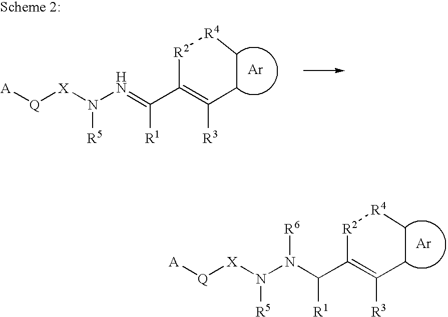

If individual compounds I are not obtainable by the route described above, they can be prepared by derivatization of other compounds I or by customary modifications of the synthesis routes described. The preparation of the compounds of formula I may lead to them being obtained as isomer mixtures (stereoisomers, enantiomers). If desired, these can be resolved by the methods customary for this purpose, such as crystallization or chromatography, also on optically active adsorbate, to give the pure isomers.

Acyl hydrazones of the formula 11 are known in the art, e.g. from PCT/EP 2004/005681, or they can be obtained applying synthesis methods described for example in WO 87/06133 by analogy. For instance, suitable hydrazides III can be reacted with aldehydes, esters or ketones IV according to scheme 3 to form acyl hydrazones of the formula II.

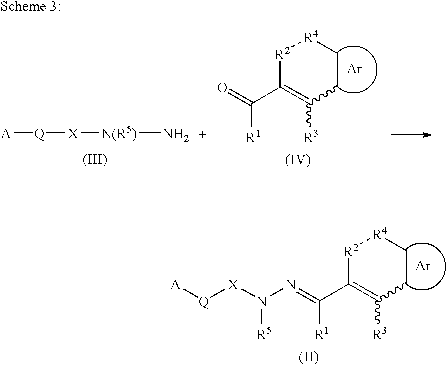

The compounds of formula I are effective through contact (via soil, glass, wall, bed net, carpet, plant parts or animal parts), and/or ingestion (bait, or plant part).

The compounds of the formula I are in particular suitable for efficiently controlling nematodes and insects. In particular, they are suitable for controlling the following pests:

Insects:

from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Cheimatobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis,

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespeftinus, Crioceris asparagi, Diabrotica longicomis, Diabrotica 12-punctata, Diabrotica virgifera, Epilachna varivestis, Epitrix hiffipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria,

dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa,

thrips (Thysanoptera), e.g. Dichromothrips spp., Frankliniella fusca, Frankliniella occidentalis, Frankliniella tritici, Scirtothrips citri, Thrips oryzae, Thrips palmi and Thrips tabaci,

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta,

heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor,

homopterans (Homoptera), e.g. Acyfthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisa tabaci, Bemisa argentifolii, Brachycaudus cardui, Brachycaudus helichrysi, Brachycaudus persicae, Brachycaudus prunicola, Brevicoryne brassicae, Capitophorus horni, Cerosipha gossypil, Chaetosiphon fragaefolli, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribisnigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum inseffum, Sappaphis mala, Sappaphis mali, Schizaphis graminum, Schizoneura lanuginosa, Sitobion avenae, Trialeurodes vaporariorum, Toxoptera aurantliand, and Viteus vitifolii,

termites (Isoptera), e.g. Calotermes flavicollis, Leucotermes flavipes, Reticulitermes lucifugus und Termes natalensis, and

orthopterans (Orthoptera), e.g. Acheta domestica, Blatta orientalis, Blattella germanica, Fofficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Nematodes:

plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifoli, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the formula I and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Omithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis.

For use in a method according to the present invention, the compounds I can be converted into the customary formulations, e.g. solutions, emulsions, suspensions, dusts, powders, pastes and granules. The use form depends on the particular purpose; it is intended to ensure in each case a fine and uniform distribution of the compound according to the invention.

The formulations are prepared in a known manner, e.g. by extending the active ingredient with solvents and/or carriers, if desired using emulsifiers and dispersants. Solvents/auxiliaries, which are suitable, are essentially:
water, aromatic solvents (for example Solvesso products, xylene), paraffins (for example mineral fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (e.g. kaolins, clays, talc, chalk) and ground synthetic minerals (e.g. highly disperse silica, silicates); emulsifiers such as nonionic and anionic emulsifiers (e.g. polyoxyethylene fatty alcohol ethers, alkylsulfonates and arylsulfonates) and dispersants such as lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

The Following are Examples of Formulations: 1. Products for Dilution with Water

A Soluble Concentrates (SL)

10 parts by weight of a compound according to the invention are dissolved in water or in a water-soluble solvent. As an alternative, wetters or other auxiliaries are added. The active ingredient dissolves upon dilution with water.

B Dispersible Concentrates (DC)

20 parts by weight of a compound according to the invention are dissolved in cyclohexanone with addition of a dispersant, for example polyvinylpyrrolidone. Dilution with water gives a dispersion.

C Emulsifiable Concentrates (EC)

15 parts by weight of a compound according to the invention are dissolved in xylene with addition of calcium dodecylbenzenesulfonate and castor oil ethoxylate (in each case 5% strength). Dilution with water gives an emulsion.

D Emulsions (EW, EO)

40 parts by weight of a compound according to the invention are dissolved in xylene with addition of calcium dodecylbenzenesulfonate and castor oil ethoxylate (in each case 5% strength). This mixture is introduced into water by means of an emulsifier (Ultraturrax) and made into a homogeneous emulsion. Dilution with water gives an emulsion.

E Suspensions (SC, OD)

In an agitated ball mill, 20 parts by weight of a compound according to the invention are milled with addition of dispersant, wetters and water or an organic solvent to give a fine active ingredient suspension. Dilution with water gives a stable suspension of the active ingredient.

F Water-Dispersible Granules and Water-Soluble Granules (WG, SG)

50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active ingredient.

G Water-Dispersible Powders and Water-Soluble Powders (WP, SP)

75 parts by weight of a compound according to the invention are ground in a rotor-stator mill with addition of dispersant, wetters and silica gel. Dilution with water gives a stable dispersion or solution with the active ingredient.

2. Products to be Applied Undiluted

H Dustable Powders (DP)

5 parts by weight of a compound according to the invention are ground finely and mixed intimately with 95% of finely divided kaolin. This gives a dustable product.

I Granules (GR, FG, GG, MG)

0.5 parts by weight of a compound according to the invention is ground finely and associated with 95.5% carriers. Current methods are extrusion, spray drying or the fluidized bed. This gives granules to be applied undiluted.

J ULV Solutions (UL)

10 parts by weight of a compound according to the invention are dissolved in an organic solvent, for example xylene. This gives a product to be applied undiluted.

The active ingredients can be used as such, in the form of their formulations or the use forms prepared therefrom, eg. in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; it is intended to ensure in each case the finest possible distribution of the active ingredients according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

Compositions of this invention may also contain other active ingredients, for example other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients. These agents usually are admixed with the agents according to the invention in a weight ratio of 1:100 to 100:1.

The following list of pesticides together with which the compounds according to the invention can be used, is intended to illustrate the possible combinations, but not to impose any limitation:

Organophosphates: Acephate, Azinphos-methyl, Chlorpyrifos, Chlorfenvinphos, Diazinon, Dichlorvos, Dicrotophos, Dimethoate, Disulfoton, Ethion, Fenitrothion, Fenthion, Isoxathion, Malathion, Methamidophos, Methidathion, Methyl-Parathion, Mevinphos, Monocrotophos, Oxydemeton-methyl, Paraoxon, Parathion, Phenthoate, Phosalone, Phosmet, Phosphamidon, Phorate, Phoxim, Pirimiphos-methyl, Profenofos, Prothiofos, Sulprophos, Tetrachlorvinphos, Terbufos, Triazophos, Trichlorfon;

Carbamates: Alanycarb, Bendiocarb, Benfuracarb, Carbaryl, Carbofuran, Carbosulfan, Fenoxycarb, Furathiocarb, Indoxacarb, Methiocarb, Methomyl, Oxamyl, Pirimicarb, Propoxur, Thiodicarb, Triazamate;

Pyrethroids: Bifenthrin, Cyfluthrin, Cypermethrin, alpha-Cypermethrin, Deltamethrin, Esfenvalerate, Ethofenprox, Fenpropathrin, Fenvalerate, Cyhalothrin, Lambda-Cyhalothrin, Permethrin, Silafluofen, Tau-Fluvalinate, Tefluthrin, Tralomethrin, Zeta-Cypermethrin;

Arthropod growth regulators: a) chitin synthesis inhibitors: benzoylureas: Chlorfluazuron, Diflubenzuron, Flucycloxuron, Flufenoxuron, Hexaflumuron, Lufenuron, Novaluron, Teflubenzuron, Triflumuron; Buprofezin, Diofenolan, Hexythiazox, Etoxazole, Clofentazine; b) ecdysone antagonists: Halofenozide, Methoxyfenozide, Tebufenozide; c) juvenoids: Pyriproxyfen, Methoprene, Fenoxycarb; d) lipid biosynthesis inhibitors: Spirodiclofen;

Various: Abamectin, Acequinocyl, Acetamiprid, Amitraz, Azadirachtin, Bifenazate, Cartap, Chlorfenapyr, Chlordimeform, Cyromazine, Diafenthiuron, Dinetofuran, Diofenolan, Emamectin, Endosulfan, Ethiprole, Fenazaquin, Fipronil, Formetanate, Formetanate hydrochloride, Hydramethylnon, Imidacloprid, lndoxacarb, Metaflumizon (=4-{(2Z)-2-({[4-(trifluoro-methoxy)anilino]carbonyl}hydrazono)-2-[3-(trifluoromethyl)-phenyl]ethyl}benzo-nitrile), Nitenpyram, Pyridaben, Pymetrozine, Spinosad, Sulfur, Tebufenpyrad, Thiamethoxam, Thiacloprid, Thiocyclam, Spiromesifen, Spirodiclofen, Pyridalyl and the pesticide of the following formula as described in WO 98/05638:

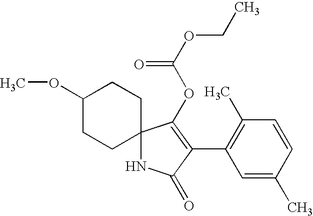

Fungicides are those selected from the group consisting of
acylalanines such as benalaxyl, metalaxyl, ofurace, oxadixyl,
amine derivatives such as aldimorph, dodine, dodemorph, fenpropimorph, fenpropidin, guazatine, iminoctadine, spiroxamin, tridemorph
anilinopyrimidines such as pyrimethanil, mepanipyrim or cyrodinyl,
antibiotics such as cycloheximid, griseofulvin, kasugamycin, natamycin, polyoxin or streptomycin,
azoles such as bitertanol, bromoconazole, cyproconazole, difenoconazole, dinitroconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol
dicarboximides such as iprodion, myclozolin, procymidon, vinclozolin,
dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,
copper fungicides such as Bordeaux mixture, copper acetate, copper oxychloride, basic copper sulfate,
nitrophenyl derivatives such as binapacryl, dinocap, dinobuton, nitrophthalisopropyl
phenylpyrroles such as fenpiclonil or fludioxonil,
sulfur
other fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid
strobilurins such as azoxystrobin, dimoxystrobin, fluoxastrobin, kresoxim-methyl, metominostrobin, orysastrobin, picoxystrobin or trifloxystrobin,
sulfenic acid derivatives such as captafol, captan, dichlofluanid, folpet, tolylfluanid
cinnemamides and analogs such as dimethomorph, flumetover or flumorph.

The aforementioned compositions are particularly useful for protecting plants against infestation of said pests or to combat these pests in infested plants. However, the compounds of formula I are also suitable for the treatment of seeds.

Compositions for seed treatments include for example flowable concentrates FS, solutions LS, powders for dry treatment DS, water dispersible powders for slurry treatment WS, water soluble powders SS and emulsion ES. Application to the seeds is carried out before sowing, either directly on the seeds or after having pregerminated the latter.

Preferred FS formulations of compounds of formula I for seed treatment usually comprise from 0.5 to 80% of the active ingredient, from 0.05 to 5% of a wetter, from 0.5 to 15% of a dispersing agent, from 0.1 to 5% of a thickener, from 5 to 20% of an anti-freeze agent, from 0.1 to 2% of an anti-foam agent, from 1 to 20% of a pigment and/or a dye, from 0 to 15% of a sticker/adhesion agent, from 0 to 75% of a filler/vehicle, and from 0.01 to 1% of a preservative.

Suitable pigments or dyes for seed treatment formulations are pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Stickers/adhesion agents are added to improve the adhesion of the active materials on the seeds after treatment. Suitable adhesives are block copolymers EO/PO surfactants but also polyvinylalcohols, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers and copolymers derived from these polymers.

Compositions which are Useful for Seed Treatment are e.g.:

A Soluble concentrates (SL, LS)

D Emulsions (EW, EO, ES)

E Suspensions (SC, OD, FS)

F Water-dispersible granules and water-soluble granules (WG, SG)

G Water-dispersible powders and water-soluble powders (WP, SP, WS)

H Dustable powders (DP, DS)

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methylneodecanamide (MNDA), a pyrethroid not used for insect control such as {(±)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is mostly done by dipping the textile material into emulsions or dispersions of the insecticide or spraying them onto the nets.

The compounds of formula I and its compositions can be used for protecting non-living material, in particular cellulose-based materials such as wooden materials e.g. trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

In the methods according to the invention the pests are controlled by contacting the target parasite/pest, its food supply, habitat, breeding ground or its locus with a pesticidally effective amount of compounds of formula I or with a salt thereof or with a composition, containing a pesticidally effective amount of a compound of formula I or a salt thereof.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of the invention can also be applied preventively to places at which occurrence of the pests is expected.

The compounds of formula I may be also used to protect growing plants from attack or infestation by pests by contacting the plant with a pesticidally effective amount of compounds of formula i. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in bait compositions, the typical content of active ingredient is from 0.001 weight % to 15 weight %, desirably from 0.001 weight % to 5% weight % of active compound.

For use in spray compositions, the content of active ingredient is from 0.001 to 80 weights %, preferably from 0.01 to 50 weight % and most preferably from 0.01 to 15 weight %.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

The products were characterized by coupled High Performance Liquid Chromatography/mass spectrometry (HPLC/MS), by NMR or by their melting points.

HPLC column: RP-18 column (Chromolith Speed ROD from Merck KgaA, Germany).

Elution: acetonitrile+0.1% trifluoroacetic acid (TFA)/water in a ratio of from 5:95 to 35 95:5 in 5 minutes at 40° C.

In the Examples the Following Abbreviations are Used:

m.p.: melting point

THF: tetrahydrofuran

MS: Quadrupol electrospray ionisation, 80 V (positiv modus)

RT: retention time

### EXAMPLE 1

### Compound A-90 of Table B

### 1.1 Furan-2-carboxylic acid N-methyl-hydrazide

To a mixture of 3.0 g (65 mmol) of methyl hydrazine in 35 ml of dichloromethane 0.85 g (6.5 mmol) of furan-2-carboxylic acid chloride in 35 ml of dichloromethane were added at 0° C. within 1 hour. The mixture was allowed to warm to room temperature and stirred for 3 hours. After evaporation of the solvent the resulting oil was sufficiently pure for further reactions (HPLC/MS: RT=0.707 min, m/z=140 [M+H]⁺).

### 1.2 Furan-2-carboxylic acid N-methyl-N′-[2-methyl-3-(2-fluorophenyl)-allylidene]-hydrazide

A mixture of 95 mg (0.66 mmol) of furan-2-carboxylic acid N-methyl-hydrazide and 98 mg (0.6 mmol) of 2-methyl-3-(2-fluorophenyl) acrolein in 5 ml of methanol was heated to reflux overnight. After cooling to room temperature and evaporation of the solvent 168 mg (0.59 mmol, 98%) of the desired product were obtained. The compound was sufficiently pure for subsequent reactions (>90% according to HPLC/MS; RT=3.640 min, m/z=287 [M+H]⁺).

### 1.3 Furan-2-carboxylic acid N′-[3-(2-fluorophenyl)-2-methyl-allyl]-N-methyl-hydrazide

To 114 mg (0.4 mmol) of furan-2-carboxylic acid N-methyl-N′-[2-methyl-3-(2-fluorophenyl)-allylidene]-hydrazide and 38 mg (0.64 mmol) of dimethylamine-borane complex in 1 ml of dichloromethane which were cooled to 0° C. 456 mg (2.4 mmol) of p-toluenesulfonic acid monohydrate in 2 ml of dichloromethand/methanol 3:1 were added. Upon stirring the resulting mixture at 0° C. for 30 min 2.5 ml of Na₂CO₃ solution (10% w/v) were added and the mixture was heated to reflux for 30 min. After cooling to room temperature the organic phase was separated and the aqueous phase was extracted twice with dichloromethane. The combined organic phases were dried over MgSO₄ and concentrated in vacuo. 95 mg (0.33 mmol, 83%) of furan-2-carboxylic acid N′-[3-(2-fluorophenyl)-2-methyl-allyl]-N-methyl-hydrazide were obtained which were >90% pure according to HPLC/MS (RT=3.217 min, m/z=289 [M+H]⁺).

### EXAMPLE 2

### Compound A-1 of Table B

### 2.1 2-Fluorobenzoic acid N′-(3-(3-fluorophenyl)-2-methyl-allylidene)hydrazide

The title compound was obtained by analogy to the method outlined in example 1.2 by reacting 2-fluorobenzoic acid hydrazide with 2-methyl-3-(3-fluorophenyl) acrolein.

### 2.2 2-Fluorobenzoic acid N′-[3-(3-fluorophenyl)-2-methyl-allyl]hydrazide

In a three-necked round-bottom flask (50 ml), fitted with a reflux condenser and a thermometer 1.00 g (3.33 mmol, 1.0 eq) of 2-fluorobenzoic acid N′-(3-(3-fluorophenyl)-2-methyl-allylidene) hydrazide and 314.7 mg (5.34 mmol, 1.6 eq) of dimethylamine-borane complex were equilibrated for 5 min in 10 ml of CH₂Cl₂ at 0° C. Then 3.804 g (20.00 mmol, 6.0 eq) of p-toluenesulfonic acid monohydrate were added as a solution in 6 ml of CH₂Cl₂/MeOH 3:1. After stirring at 0° for 30 minutes 12.5 ml of Na₂CO₃ solution (10% w/v) and 4 ml MeOH were added and the mixture was refluxed for a further 30 minutes.

After cooling to room temperature the organic phase was separated and the aqueous phase was extracted twice with CH₂Cl₂. The combined organic phases were dried over MgSO₄ and concentrated in vacuo. 0.956 g (3.17 mmol, 95%) of 2-fluorobenzoic acid N′-(3-(3-fluorophenyl)-2-methyl-allyl) hydrazide were obtained which were >95% pure according to HPLC/MS (¹H-NMR, d₆-DMSO, 500 MHz; δ=1.9 (s, 3H), 3.5 (d, 2H), 5.5 (pt, 1H), 6.5 (s, 1H), 7.0-7.15 (m, 3H), 7.2-7.3 (m, 2H), 7.3-7.4 (m, 1H), 7.4-7.55 (m, 2H), 9.75 (d, 1H)).

The compounds A2 to A321 given in table B, compounds B1 to B56 given in table C, compounds C1 and C2 given in table D, compounds D1 and D2 given in table E, compounds E1 to E38 given in table F, compound F1 given in table G, compounds G1 to G3 given in table H, compounds H1 to H55 given in table J, compounds J1 to J26 given in table K and compounds K1 to K10 given in table L were obtained by analogy to the methods of examples 1 and 2.

TABLE B

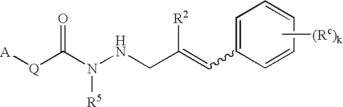

| No. | A | Q | R² | R⁵ | (R^{c})ₖ | RT (HPLC/MS) |
|---|---|---|---|---|---|---|
| A1 | 2-fluorophenyl | — | CH₃ | H | 3-F | 2.679 min; m/z = 303 [M + H]⁺ |
| A2 | 2-pyridyl | — | CH₃ | H | — | 2.634 min; m/z = 268 [M + H]⁺ |
| A3 | 2-pyridyl | — | OCH₃ | H | — | 2.759 min; m/z = 284 [M + H]⁺ |
| A4 | 6-bromo-2-pyridyl | — | OCH₃ | H | — | 3.262 min; m/z = 362 [M + H]⁺ |
| A5 | 2-furyl | — | OCH₃ | H | — | 2.659 min; m/z = 273 [M + H]⁺ |
| A6 | 2-thienyl | — | OCH₃ | H | — | 2.857 min; m/z = 289 [M + H]⁺ |
| A7 | 2,4-difluorophenyl | — | OCH₃ | H | — | 3.127 min; m/z = 319 [M + H]⁺ |
| A8 | 2-fluorophenyl | — | OCH₃ | H | — | 3.009 min; m/z = 301 [M + H]⁺ |
| A9 | phenyl | CH₂ | OCH₃ | H | — | 2.939 min; m/z = 297 [M + H]⁺ |
| A10 | 5-methyl-2-pyrazinyl | — | CH₃ | H | 4-F | 2.731 min; m/z = 323 [M + Na]⁺ |
| A11 | 5-methyl-2-pyrazinyl | — | CH₃ | H | 2-F | 2.707 min; m/z = 301 [M + H]⁺ |
| A12 | 6-fluoro-2-pyridyl | — | CH₃ | H | 4-F | 3.020 min; m/z = 326 [M + Na]⁺ |
| A13 | 6-fluoro-2-pyridyl | — | CH₃ | H | 3-F | 3.051 min; m/z = 304 [M + H]⁺ |
| A14 | 3-hydroxy-2-naphthyl | — | CH₃ | H | — | 3.393 min; m/z = 333 [M + H]⁺ |
| A15 | 3-pyridyl | — | CH₃ | H | — | 2.188 min; m/z = 268 [M + H]⁺ |
| A16 | 4-pyridyl | — | CH₃ | H | — | 2.163 min; m/z = 268 [M + H]⁺ |
| A17 | 4-methylphenyl | — | CH₃ | H | — | 2.935 min; m/z = 281 [M + H]⁺ |
| A18 | 2-nitrophenyl | — | CH₃ | H | — | 3.007 min; m/z = 334 [M + Na]⁺ |
| A19 | 3-chlorophenyl | — | CH₃ | H | — | 3.202 min; m/z = 323 [M + Na]⁺ |
| A20 | 4-trifluoromethylphenyl | — | CH₃ | H | — | 3.415 min; m/z = 357 [M + Na]⁺ |
| A21 | 2-furyl | — | CH₃ | H | — | 2.617 min; m/z = 257 [M + H]⁺ |
| A22 | 3-methoxyphenyl | — | CH₃ | H | — | 2.857 min; m/z = 297 [M + H]⁺ |
| A23 | 4-chlorophenyl | — | CH₃ | H | — | 3.169 min; m/z = 323 [M + Na]⁺ |
| A24 | 4-nitrophenyl | — | CH₃ | H | — | 3.076 min; m/z = 334 [M + Na]⁺ |
| A25 | 2-chlorophenyl | — | CH₃ | H | — | 3.076 min; m/z = 301 [M + H]⁺ |
| A26 | 2,5-dichlorophenyl | — | CH₃ | H | — | 3.435 min; m/z = 357 [M + Na]⁺ |
| A27 | 2-methoxyphenyl | — | CH₃ | H | — | 2.889 min; m/z = 297 [M + H]⁺ |
| A28 | 3,4,5-trimethoxyphenyl | — | CH₃ | H | — | 2.805 min; m/z = 357 [M + H]⁺ |
| A29 | 4-tert-butylphenyl | — | CH₃ | H | — | 3.464 min; m/z = 323 [M + H]⁺ |
| A30 | 3-nitrophenyl | — | CH₃ | H | — | 3.065 min; m/z = 334 [M + Na]⁺ |
| A31 | 3,4-dimethoxyphenyl | — | CH₃ | H | — | 2.622 min; m/z = 327 [M + H]⁺ |
| A32 | 4-methoxyphenyl | — | CH₃ | H | — | 2.741 min; m/z = 297 [M + H]⁺ |
| A33 | 3,4-dimethoxyphenyl | CH₂ | CH₃ | H | — | 2.580 min; m/z = 341 [M + H]⁺ |
| A34 | 3,5-bis- | — | CH₃ | H | — | 3.942 min; m/z = 425 [M + Na]⁺ |
| | (trifluoromethyl)phenyl | | | | | |
| A35 | 2,4-dichlorophenyl | OCH(CH₃) | CH₃ | H | — | 3.811 min; m/z = 401 [M + Na]⁺ |
| A36 | 3-methyl-4-nitrophenyl | — | CH₃ | H | — | 3.222 min; m/z = 348 [M + Na]⁺ |
| A37 | 2-thienyl | — | CH₃ | H | — | 2.770 min; m/z = 273 [M + H]⁺ |
| A38 | 3-methyl-imidazol-2-yl | — | CH₃ | H | — | 2.109 min; m/z = 271 [M + H]⁺ |
| A39 | 2-benzothienyl | — | CH₃ | H | — | 3.377 min; m/z = 323 [M + H]⁺ |
| A40 | 2-phenoxypyrid-3-yl | — | CH₃ | H | — | 3.351 min; m/z = 360 [M + H]⁺ |
| A41 | 2-hydroxyphenyl | — | CH₃ | H | — | 2.957 min; m/z = 283 [M + H]⁺ |
| A42 | 3-trifluoromethyphenyl | | CH₃ | H | — | 3.378 min; m/z = 357 [M + Na]⁺ |
| A43 | 4-hydroxyphenyl | — | CH₃ | H | — | 2.357 min; m/z = 283 [M + H]⁺ |
| A44 | phenyl | — | CH₃ | H | — | 2.754 min; m/z = 267 [M + H]⁺ |
| A45 | 3-hydroxyphenyl | — | CH₃ | H | — | 2.473 min; m/z = 283 [M + H]⁺ |
| A46 | 3-chloro-2-benzothienyl | — | CH₃ | H | — | 3.836 min; m/z = 357 [M + H]⁺ |
| A47 | 4-dimethylaminophenyl | — | CH₃ | H | — | 2.696 min; m/z = 310 [M + H]⁺ |
| A48 | benzo-[1,3]-dioxol-5-yl | — | CH₃ | H | — | 2.729 min; m/z = 311 [M + H]⁺ |
| A49 | 3-ethoxyphenyl | — | CH₃ | H | — | 3.068 min; m/z = 311 [M + H]⁺ |
| A50 | 4-fluorophenyl | — | CH₃ | H | — | 2.888 min; m/z = 285 [M + H]⁺ |
| A51 | 2-fluorophenyl | — | CH₃ | H | — | 2.966 min; m/z = 285 [M + H]⁺ |
| A52 | 3-fluorophenyl | — | CH₃ | H | — | 2.969 min; m/z = 307 [M + Na]⁺ |
| A53 | 2,6-dimethylphenyl | NHCH₂CH₂ | CH₃ | H | — | 2.613 min; m/z = 338 [M + H]⁺ |
| A54 | 4-bromophenyl | — | CH₃ | H | — | 3.202 min; m/z = 367 [M + Na]⁺ |
| A55 | 2,4-dichlorophenyl | — | CH₃ | H | — | 3.429 min; m/z = 357 [M + Na]⁺ |
| A56 | 4-aminophenyl | — | CH₃ | H | — | 2.228 min; m/z = 282 [M + H]⁺ |
| A57 | 6-amino-2-fluoro-phenyl | — | CH₃ | H | — | 2.847 min; m/z = 300 [M + H]⁺ |
| A58 | 4-amino-2-methyl- | — | CH₃ | H | — | 2.130 min; m/z = 298 [M + H]⁺ |
| | pyrimid-5-yl | | | | | |
| A59 | 3-aminophenyl | — | CH₃ | H | — | 2.180 min; m/z = 282 [M + H]⁺ |
| A60 | 4-chlorophenyl | NHCOCH₂ | CH₃ | H | — | 3.144 min; m/z = 358 [M + H]⁺ |
| A61 | 4-hydroxy-3- | — | CH₃ | H | — | 2.451 min; m/z = 313 [M + H]⁺ |
| | methoxyphenyl | | | | | |
| A62 | 2-trifluoromethylphenyl | — | CH₃ | H | — | 3.280 min; m/z = 357 [M + Na]⁺ |
| A63 | 3-ethylisoxazol-5-yl | — | CH₃ | H | — | 3.029 min; m/z = 308 [M + Na]⁺ |
| A64 | 3-methoxyphenyl | CH₂ | CH₃ | H | — | 2.898 min; m/z = 311 [M + H]⁺ |
| A65 | 2-pyridyl | CH₂ | CH₃ | H | — | 1.969 min; m/z = 282 [M + H]⁺ |
| A66 | 5-chloromethylisoxazol- | — | CH₃ | H | — | 3.133 min; m/z = 328 [M + Na]⁺ |
| | 3-yl | | | | | |
| A67 | 3,6-dichloro-2-pyridyl | — | CH₃ | H | — | 3.246 min; m/z = 336 [M + H]⁺ |
| A68 | 2-methoxyphenyl | CH₂ | CH₃ | H | — | 2.932 min; m/z = 311 [M + H]⁺ |
| A69 | 5-nitro-2-pyridyl | — | CH₃ | H | — | 3.149 min; m/z = 335 [M + Na]⁺ |
| A70 | 3-methoxy-2-thienyl | — | CH₃ | H | — | 2.880 min; m/z = 303 [M + H]⁺ |
| A71 | 6-methyl-2-pyridyl | — | CH₃ | H | — | 2.978 min; m/z = 282 [M + H]⁺ |
| A72 | 1,3-bis(1,1,1-trifluoro-2- | — | CH₃ | H | — | 3.375 min; m/z = 429 [M + Na]⁺ |
| | ethyl)-pyrazol-4-yl | | | | | |
| A73 | 4-chloro-2-pyridyl | — | CH₃ | H | — | 3.261 min; m/z = 302 [M + H]⁺ |
| A74 | 4-pyridyl | CH₂ | CH₃ | H | — | 1.816 min; m/z = 282 [M + H]⁺ |
| A75 | 3-pyridyl | CH₂ | CH₃ | H | — | 1.874 min; m/z = 282 [M + H]⁺ |
| A76 | 6-hydroxy-2-methyl-4- | — | CH₃ | H | — | 2.408 min; m/z = 299 [M + H]⁺ |
| | pyrimidyl | | | | | |
| A77 | 3-methyl-5-isoxazolyl | — | CH₃ | H | — | 2.797 min; m/z = 294 [M + Na]⁺ |
| A78 | phenyl | — | CH₃ | CH₃ | 4-F | 3.445 min; m/z = 299 [M + H]⁺ |
| A79 | phenyl | — | Cl | CH₃ | — | 3.369 min; m/z = 301 [M + H]⁺ |
| A80 | phenyl | — | H | CH₃ | — | 3.118 min; m/z = 267 [M + H]⁺ |
| A81 | phenyl | — | CH₃ | CH₃ | — | 3.390 min; m/z = 281 [M + H]⁺ |
| A82 | phenyl | — | C₂H₅ | CH₃ | — | 3.622 min; m/z = 295 [M + H]⁺ |
| A83 | phenyl | CH₂ | CH₃ | CH₃ | 4-F | 3.552 min; m/z = 313 [M + H]⁺ |
| A84 | phenyl | CH₂ | H | CH₃ | — | 3.260 min; m/z = 281 [M + H]⁺ |
| A85 | 1-methyl-1H-pyridin-2- | — | CH₃ | CH₃ | 4-F | 2.658 min; m/z = 330 [M + H]⁺ |
| | on-3-yl | | | | | |
| A86 | 1-methyl-1H-pyridin-2- | — | CH₃ | CH₃ | 2-F | 2.616 min; m/z = 330 [M + H]⁺ |
| | on-3-yl | | | | | |
| A87 | 1-methyl-1H-pyridin-2- | — | H | CH₃ | — | 2.333 min; m/z = 298 [M + H]⁺ |
| | on-3-yl | | | | | |
| A88 | 2-furyl | — | CH₃ | CH₃ | 3-F | 3.186 min; m/z = 289 [M + H]⁺ |
| A89 | 2-furyl | — | CH₃ | CH₃ | 4-F | 3.185 min; m/z = 289 [M + H]⁺ |
| A90 | 2-furyl | — | CH₃ | CH₃ | 2-F | 3.153 min; m/z = 289 [M + H]⁺ |
| A91 | 2-furyl | — | H | CH₃ | — | 2.891 min; m/z = 257 [M + H]⁺ |
| A92 | 2-furyl | — | CH₃ | CH₃ | — | 3.127 min; m/z = 271 [M + H]⁺ |
| A93 | phenyl | — | CH₃ | CH₃ | 3-F | 3.404 min; m/z = 299 [M + H]⁺ |
| A94 | phenyl | — | CH₃ | CH₃ | 2-F | 3.380 min; m/z = 299 [M + H]⁺ |
| A95 | phenyl | — | Br | CH₃ | — | 3.398 min; m/z = 346 [M + H]⁺ |
| A96 | 3-methylphenyl | — | CH₃ | H | — | 2.937 min; m/z = 281 [M + H]⁺ |
| A97 | phenyl | CH₂ | CH₃ | H | — | 2.823 min; m/z = 281 [M + H]⁺ |
| A98 | 1-naphthyl | CH₂ | CH₃ | H | — | 3.193 min; m/z = 331 [M + H]⁺ |
| A99 | 2-methylphenyl | — | CH₃ | H | — | 2.907 min; m/z = 281 [M + H]⁺ |
| A100 | 1-naphthyl | — | CH₃ | H | — | 3.154 min; m/z = 317 [M + H]⁺ |
| A101 | 4-ethoxyphenyl | — | CH₃ | H | — | 2.948 min; m/z = 311 [M + H]⁺ |
| A102 | 4-phenylphenyl | — | CH₃ | H | — | 3.421 min; m/z = 343 [M + H]⁺ |
| A103 | 2-naphthyl | — | CH₃ | H | — | 3.240 min; m/z = 339 [M + Na]⁺ |
| A104 | 5-bromo-2-chlorophenyl | — | CH₃ | H | — | 3.438 min; m/z = 403 [M + Na]⁺ |
| A105 | 4-fluorophenyl | OCH₂ | CH₃ | H | — | 3.202 min; m/z = 337 [M + Na]⁺ |
| A106 | 2-methyl-4-chlorophenyl | OCH₂ | CH₃ | H | — | 3.638 min; m/z = 367 [M + Na]⁺ |
| A107 | 3-bromophenyl | — | CH₃ | H | — | 3.280 min; m/z = 367 [M + Na]⁺ |
| A108 | 2-chlorophenyl | OCH₂ | CH₃ | H | — | 3.430 min; m/z = 353 [M + Na]⁺ |
| A109 | 3-indolyl | CH₂ | CH₃ | H | — | 2.765 min; m/z = 320 [M + H]⁺ |
| A110 | 2-benzimidazolyl | SCH₂ | CH₃ | H | — | 2.519 min; m/z = 353 [M + H]⁺ |
| A111 | 4-methylsulfonyl-2- | — | CH₃ | H | — | 2.969 min; m/z = 412 [M + Na]⁺ |
| | nitrophenyl | | | | | |
| A112 | 6,7-dimethoxy-3-methyl- | — | CH₃ | H | — | 2.381 min; m/z = 397 [M + H]⁺ |
| | 1,2,3,4-tetrahydro- | | | | | |
| | isoquinolin-3-yl | | | | | |
| A113 | 4-chlorophenyl | OCH₂ | CH₃ | H | — | 3.397 min; m/z = 353 [M + Na]⁺ |
| A114 | 6-hydroxy-2-pyridyl | — | CH₃ | H | — | 2.362 min; m/z = 306 [M + Na]⁺ |
| A115 | 2-chloro-6-fluorophenyl | CH₂ | CH₃ | H | — | 3.113 min; m/z = 355 [M + Na]⁺ |
| A116 | 2-pyrazinyl | — | CH₃ | H | — | 2.583 min; m/z = 291 [M + Na]⁺ |
| A117 | 2-fluoro-4-hydroxyphenyl | — | CH₃ | H | — | 2.598 min; m/z = 323 [M + Na]⁺ |
| A118 | 3-fluoro-2-pyridyl | — | CH₃ | H | — | 2.688 min; m/z = 308 [M + Na]⁺ |
| A119 | 3-methoxy-2-pyridyl | — | CH₃ | H | — | 2.371 min; m/z = 320 [M + Na]⁺ |
| A120 | phenyl | CH₂ | CH₃ | CH₃ | 3-F | 3.562 min; m/z = 313 [M + H]⁺ |
| A121 | phenyl | CH₂ | CH₃ | CH₃ | 2-F | 3.543 min; m/z = 313 [M + H]⁺ |
| A122 | phenyl | OCH₂ | H | CH₃ | — | 3.329 min; m/z = 297 [M + H]⁺ |
| A123 | phenyl | SCH₂ | CH₃ | CH₃ | 4-F | 3.780 min; m/z = 367 [M + Na]⁺ |
| A124 | phenyl | OCH₂ | CH₃ | H | — | 3.093 min; m/z = 297 [M + H]⁺ |
| A125 | 4-chloro-2-methylphenyl | OCH(CH₃) | CH₃ | H | — | 3.714 min; m/z = 359 [M + H]⁺ |
| A126 | 2,4-dichlorophenyl | O(CH₂)₃ | CH₃ | H | — | 3.502 min; m/z = 393 [M + H]⁺ |
| A127 | 4-fluoro 2-trifluoromethyl- | — | CH₃ | H | — | 3.332 min; m/z = 375 [M + Na]⁺ |
| | phenyl | | | | | |
| A128 | 2,6,dichlorophenyl | CH₂ | CH₃ | H | — | 3.199 min; m/z = 349 [M + H]⁺ |
| A129 | 6-bromo-2-pyridyl | — | CH₃ | H | — | 3.234 min; m/z = 346 [M + H]⁺ |
| A130 | 4-bromophenyl | CH₂ | CH₃ | H | — | 3.135 min; m/z = 381 [M + Na]⁺ |
| A131 | 6-trifluoromethyl-2- | — | CH₃ | H | — | 3.382 min; m/z = 358 [M + Na]⁺ |
| | pyridyl | | | | | |
| A132 | 5-trifluoromethyl-2- | — | CH₃ | H | — | 3.410 min; m/z = 358 [M + Na]⁺ |
| | pyridyl | | | | | |
| A133 | 4-hydroxyphenyl | CH₂ | CH₃ | H | — | 2.290 min; m/z = 297 [M + H]⁺ |
| A134 | 5-methylisoxazol-3-yl | — | CH₃ | H | — | 2.845 min; m/z = 294 [M + Na]⁺ |
| A135 | 3-fluorophenyl | CH₂ | CH₃ | H | — | 2.882 min; m/z = 299 [M + H]⁺ |
| A136 | 2-fluorophenyl | CH₂ | CH₃ | H | — | 2.847 min; m/z = 299 [M + H]⁺ |
| A137 | 2-thienyl | CH₂ | CH₃ | H | — | 2.766 min; m/z = 287 [M + H]⁺ |
| A138 | 4-methoxyphenyl | CH₂ | CH₃ | H | — | 2.733 min; m/z = 311 [M + H]⁺ |
| A139 | 3-amino-2-pyrazinyl | — | CH₃ | H | — | 2.564 min; m/z = 284 [M + H]⁺ |
| A140 | 3-hydroxy-2-pyridyl | — | CH₃ | H | — | 3.014 min; m/z = 284 [M + H]⁺ |
| A141 | 2,4-difluorophenyl | — | CH₃ | H | — | 3.050 min; m/z = 325 [M + Na]⁺ |
| A142 | 3-chlorophenyl | CH₂ | CH₃ | H | — | 3.078 min; m/z = 315 [M + H]⁺ |
| A143 | 5-isoxazolyl | — | CH₃ | H | — | 2.621 min; m/z = 280 [M + Na]⁺ |
| A144 | 3,4-dichlorophenyl | CH₂ | CH₃ | H | — | 3.300 min; m/z = 371 [M + Na]⁺ |
| A145 | 5-hydroxymethyl-3- | — | CH₃ | H | — | 2.440 min; m/z = 310 [M + Na]⁺ |
| | isoxazolyl | | | | | |
| A146 | 4-fluorophenyl | CH₂ | CH₃ | H | — | 2.855 min; m/z = 299 [M + H]⁺ |
| A147 | 4-chlorophenyl | CH₂ | CH₃ | H | — | 3.064 min; m/z = 315 [M + H]⁺ |
| A148 | 5-bromo-2-furyl | — | CH₃ | H | — | 3.017 min; m/z = 358 [M + Na]⁺ |
| A149 | 2-chloro-4-pyridyl | — | CH₃ | H | — | 2.906 min; m/z = 302 [M + H]⁺ |
| A150 | 1-methyl-3- | — | CH₃ | H | — | 3.366 min; m/z = 361 [M + Na]⁺ |
| | trifluoromethyl-5- | | | | | |
| | pyrazolyl | | | | | |
| A151 | 3-methyl-2-thienyl | — | CH₃ | H | — | 2.853 min; m/z = 287 [M + H]⁺ |
| A152 | 2-methyl-3-furyl | — | CH₃ | H | — | 2.659 min; m/z = 271 [M + H]⁺ |
| A153 | 2-bromo-4,5-dimethyl-3- | — | CH₃ | H | — | 3.441 min; m/z = 380 [M + H]⁺ |
| | thienyl | | | | | |
| A154 | 3-chloro-2-thienyl | — | CH₃ | H | — | 3.157 min; m/z = 307 [M + H]⁺ |
| A155 | 2,6-dichloro-4-pyridyl | — | CH₃ | H | — | 3.425 min; m/z = 358 [M + Na]⁺ |
| A156 | 5,6-dichloro-3-pyridyl | — | CH₃ | H | — | 3.298 min; m/z = 358 [M + Na]⁺ |
| A157 | 6-chloro-3-pyridyl | — | CH₃ | H | — | 2.825 min; m/z = 302 [M + H]⁺ |
| A158 | 5-bromo-3-pyridyl | — | CH₃ | H | — | 2.903 min; m/z = 368 [M + Na]⁺ |
| A159 | 2,5-difluorophenyl | — | CH₃ | H | — | 3.102 min; m/z = 325 [M + Na]⁺ |
| A160 | 3-fluoro-4-methylphenyl | — | CH₃ | H | — | 3.081 min; m/z = 299 [M + H]⁺ |
| A161 | 2-methoxy-3- | — | CH₃ | H | — | 3.098 min; m/z = 343 [M + H]⁺ |
| | methylthiophenyl | | | | | |
| A162 | 6-fluoro-2-pyridyl | — | CH₃ | H | — | 2.973 min; m/z = 286 [M + H]⁺ |
| A163 | 6-chloro-2-pyridyl | — | CH₃ | H | — | 3.173 min; m/z = 302 [M + H]⁺ |
| A164 | 6-methoxy-2-pyridyl | — | CH₃ | H | — | 3.059 min; m/z = 298 [M + H]⁺ |
| A165 | phenyl | CH₂ | CH₃ | CH₃ | — | 3.510 min; m/z = 295 [M + H]⁺ |
| A166 | phenyl | OCH₂ | CH₃ | CH₃ | 3-F | 3.524 min; m/z = 329 [M + H]⁺ |
| A167 | phenyl | CH₂ | Cl | CH₃ | — | 3.516 min; m/z = 315 [M + H]⁺ |
| A168 | 1-methyl-1H-pyridin-2- | — | C₂H₅ | CH₃ | — | 2.787 min; m/z = 326 [M + H]⁺ |
| | on-3-yl | | | | | |
| A169 | 2-furyl | — | Cl | CH₃ | — | 3.117 min; m/z = 291 [M + H]⁺ |
| A170 | 2-furyl | — | C₂H₅ | CH₃ | — | 3.363 min; m/z = 285 [M + H]⁺ |
| A171 | phenyl | SCH₂ | H | CH₃ | — | 3.499 min; m/z = 313 [M + H]⁺ |
| A172 | phenyl | SCH₂ | CH₃ | CH₃ | — | 3.706 min; m/z = 327 [M + H]⁺ |
| A173 | phenyl | CH₂CH₂ | CH₃ | CH₃ | 3-F | 3.694 min; m/z = 327 [M + H]⁺ |
| A174 | phenyl | CH₂CH₂ | CH₃ | CH₃ | 4-F | 3.687 min; m/z = 327 [M + H]⁺ |
| A175 | phenyl | CH₂CH₂ | CH₃ | CH₃ | 2-F | 3.667 min; m/z = 327 [M + H]⁺ |
| A176 | phenyl | CH₂CH₂ | H | CH₃ | — | 3.406 min; m/z = 295 [M + H]⁺ |
| A177 | phenyl | CH₂CH₂ | CH₃ | CH₃ | — | 3.647 min; m/z = 309 [M + H]⁺ |
| A178 | 1-methyl-1H-pyridin-2- | — | CH₃ | CH₃ | 3-F | 2.661 min; m/z = 330 [M + H]⁺ |
| | on-3-yl | | | | | |
| A179 | 1-methyl-1H-pyridin-2- | — | CH₃ | CH₃ | — | 2.581 min; m/z = 312 [M + H]⁺ |
| | on-3-yl | | | | | |
| A180 | 5-isoxazolyl | — | CH₃ | CH₃ | 3-F | 3.063 min; m/z = 290 [M + H]⁺ |
| A181 | 6-phenyl-2-pyridyl | — | CH₃ | H | — | 3.576 min; m/z = 344 [M + H]⁺ |
| A182 | 3-isopropyl-5-isoxazolyl | — | CH₃ | H | — | 3.206 min; m/z = 300 [M + H]⁺ |
| A183 | 3-ethoxy-5-trifluormethyl- | — | CH₃ | H | — | 3.838 min; m/z = 385 [M + H]⁺ |
| | 2-thienyl | | | | | |
| A184 | 2-chlorophenyl | CH₂ | CH₃ | H | — | 2.999 min; m/z = 315 [M + H]⁺ |
| A185 | 2-trifluoromethylphenyl | CH₂ | CH₃ | H | — | 3.189 min; m/z = 349 [M + H]⁺ |
| A186 | 3-trifluoromethylphenyl | CH₂ | CH₃ | H | — | 3.255 min; m/z = 371 [M + Na]⁺ |
| A187 | 2-chloro-4-fluorophenyl | CH₂ | CH₃ | H | — | 3.091 min; m/z = 333 [M + H]⁺ |
| A188 | 5-chloro-2-thienyl | — | CH₃ | H | — | 3.316 min; m/z = 329 [M + Na]⁺ |
| A189 | 3-methoxy-5- | — | CH₃ | H | — | 3.598 min; m/z = 371 [M + H]⁺ |
| | trifluoromethyl-2-thienyl | | | | | |
| A190 | 2,3-dichlorophenyl | CH₂ | CH₃ | H | — | 3.236 min; m/z = 349 [M + H]⁺ |
| A191 | 5-isoxazolyl | — | CH₃ | CH₃ | 4-F | 3.068 min; m/z = 312 [M + Na]⁺ |
| A192 | 5-isoxazolyl | — | CH₃ | CH₃ | 2-F | 3.027 min; m/z = 290 [M + H]⁺ |
| A193 | 2-fluorophenyl | — | nC₅H₁₁ | H | — | 3.893 min; m/z = 341 [M + H]⁺ |
| A194 | 2-fluorophenyl | — | nC₆H₁₃ | H | — | 4.114 min; m/z = 355 [M + H]⁺ |
| A195 | 2-fluorophenyl | — | Br | H | — | 3.185 min; m/z = 350 [M + H]⁺ |
| A196 | 2-fluorophenyl | — | H | H | 4-NO₂ | 2.752 min; m/z = 316 [M + H]⁺ |
| A197 | 2-fluorophenyl | — | H | H | 4-N(CH₃)₂ | 1.778 min; m/z = 314 [M + H]⁺ |
| A198 | 2-fluorophenyl | — | H | H | 2-NO₂ | 2.665 min; m/z = 316 [M + H]⁺ |
| A199 | 2-fluorophenyl | — | CH₃ | H | 2,4-Cl₂ | 3.612 min; m/z = 353 [M + H]⁺ |
| A200 | 2-fluorophenyl | — | H | H | 3-NO₂ | 2.733 min; m/z = 316 [M + H]⁺ |
| A201 | 2-fluorophenyl | — | CH₃ | H | 4-CH₃ | 3.149 min; m/z = 321 [M + Na]⁺ |
| A202 | 2-fluorophenyl | — | H | H | 3,5-Cl₂ | 3.406 min; m/z = 339 [M + H]⁺ |
| A203 | 2-fluorophenyl | — | C₂H₅ | H | — | 3.201 min; m/z = 299 [M + H]⁺ |
| A204 | 2-fluorophenyl | — | H | H | 4-OCH₃ | 2.586 min; m/z = 323 [M + Na]⁺ |
| A205 | 2-fluorophenyl | — | H | H | 4-Br | 3.069 min; m/z = 371 [M + Na]⁺ |
| A206 | 2-fluorophenyl | — | CH₃ | H | 4-NO₂ | 3.025 min; m/z = 330 [M + H]⁺ |
| A207 | 2-fluorophenyl | — | CH₃ | H | 4-tC₄H₉ | 3.719 min; m/z = 363 [M + Na]⁺ |
| A208 | 2-fluorophenyl | — | nC₃H₇ | H | 2,4-Cl₂ | 4.065 min; m/z = 381 [M + H]⁺ |
| A209 | 2-fluorophenyl | — | H | H | 3-OCH₃ | 2.658 min; m/z = 301 [M + H]⁺ |
| A210 | 2-fluorophenyl | — | H | H | 4-OC₂H₅ | 2.829 min; m/z = 337 [M + Na]⁺ |
| A211 | 2-fluorophenyl | — | C₂H₅ | H | 4-tC₄H₉ | 3.963 min; m/z = 377 [M + Na]⁺ |
| A212 | 2-fluorophenyl | — | C₂H₅ | H | 4-OCH₃ | 3.107 min; m/z = 351 [M + Na]⁺ |
| A213 | 2-fluorophenyl | — | CH₃ | H | 4-OCH₃ | 2.854 min; m/z = 337 [M + Na]⁺ |
| A214 | 2-fluorophenyl | — | CH₃ | H | 4-Cl | 3.303 min; m/z = 319 [M + H]⁺ |
| A215 | 2-fluorophenyl | — | H | H | 2-CH₃ | 2.826 min; m/z = 307 [M + Na]⁺ |
| A216 | 2-fluorophenyl | — | H | H | 3-F | 2.777 min; m/z = 289 [M + H]⁺ |
| A217 | 2-fluorophenyl | — | H | H | 4-CN | 2.567 min; m/z = 296 [M + H]⁺ |
| A218 | 2-fluorophenyl | — | H | H | 2-F | 2.754 min; m/z = 289 [M + H]⁺ |
| A219 | 2-fluorophenyl | — | H | H | — | 2.646 min; m/z = 271 [M + H]⁺ |
| A220 | 2-fluorophenyl | — | Cl | H | — | 3.122 min; m/z = 305 [M + H]⁺ |
| A221 | 2-fluorophenyl | — | H | H | 2-OCH₃ | 2.658 min; m/z = 323 [M + Na]⁺ |
| A222 | 2-fluorophenyl | — | H | H | 4-OnC₆H₁₃ | 3.840 min; m/z = 393 [M + Na]⁺ |
| A223 | 2-fluorophenyl | — | 4- | H | 3,5-Cl₂ | 4.123 min; m/z = 433 [M + H]⁺ |
| | | | fluorophenyl | | | |
| A224 | 2-fluorophenyl | — | H | H | 3-CH═CH—CH═CH-4 | 3.114 min; m/z = 343 [M + Na]⁺ |
| A225 | 2-fluorophenyl | — | H | H | 2-CH═CH—CH═CH-3 | 3.077 min; m/z = 343 [M + Na]⁺ |
| A226 | 2-fluorophenyl | — | H | H | 2-C₂H₅ | 3.027 min; m/z = 321 [M + Na]⁺ |
| A227 | 2-fluorophenyl | — | 4- | H | 2,4-Cl₂ | 4.286 min; m/z = 449 [M + H]⁺ |
| | | | chlorophenyl | | | |
| A228 | 2-fluorophenyl | — | phenyl | H | 2-Cl-5-NO₂ | 3.654 min; m/z = 426 [M + H]⁺ |
| A229 | 2-fluorophenyl | — | H | H | 2-Cl | 2.953 min; m/z = 305 [M + H]⁺ |
| A230 | 2-fluorophenyl | — | phenyl | H | 2,4-Cl₂ | 4.055 min; m/z = 415 [M + H]⁺ |
| A231 | 2-fluorophenyl | — | 4- | H | 4-Cl | 3.859 min; m/z = 399 [M + H]⁺ |
| | | | fluorophenyl | | | |
| A232 | phenyl | CH₂ | C₂H₅ | CH₃ | — | 3.740 min; m/z = 309 [M + H]⁺ |
| A233 | phenyl | OCH₂ | CH₃ | CH₃ | 4-F | 3.537 min; m/z = 329 [M + H]⁺ |
| A234 | phenyl | OCH₂ | Cl | CH₃ | — | 3.456 min; m/z = 331 [M + H]⁺ |
| A235 | phenyl | OCH₂ | CH₃ | CH₃ | 2-F | 3.545 min; m/z = 329 [M + H]⁺ |
| A236 | phenyl | OCH₂ | CH₃ | CH₃ | — | 3.551 min; m/z = 311 [M + H]⁺ |
| A237 | phenyl | OCH₂ | C₂H₅ | CH₃ | — | 3.737 min; m/z = 325 [M + H]⁺ |
| A238 | phenyl | SCH₂ | CH₃ | CH₃ | 3-F | 3.801 min; m/z = 345 [M + H]⁺ |
| A239 | 1,2,3,4,5- | CH₂ | CH₃ | H | — | 3.364 min; m/z = 393 [M + Na]⁺ |
| | pentafluorophenyl | | | | | |
| A240 | 2,6-dichloro-3-pyridyl | — | CH₃ | H | — | 3.188 min; m/z = 358 [M + Na]⁺ |
| A241 | 4-trifluoromethyl-2- | — | CH₃ | H | — | 2.977 min; m/z = 336 [M + H]⁺ |
| | pyridyl | | | | | |
| A242 | 2,6-dichloro-4-methyl-3- | — | CH₃ | H | — | 3.285 min; m/z = 372 [M + H]⁺ |
| | pyridyl | | | | | |
| A243 | 2-fluorophenyl | — | phenyl | H | 4-Cl | 3.821 min; m/z = 403 [M + Na]⁺ |
| A244 | 2-fluorophenyl | — | 4- | H | 2-Cl | 3.730 min; m/z = 399 [M + H]⁺ |
| | | | fluorophenyl | | | |
| A245 | 2-fluorophenyl | — | H | H | 4-Cl | 2.992 min; m/z = 327 [M + Na]⁺ |
| A246 | 2-fluorophenyl | — | phenyl | H | 2,6-Cl₂ | 3.762 min; m/z = 415 [M + H]⁺ |
| A247 | 2-fluorophenyl | — | H | H | 4-C₆H₅ | 3.362 min; m/z = 369 [M + Na]⁺ |
| A248 | 2-fluorophenyl | — | H | H | 4-C₂H₅ | 3.094 min; m/z = 321 [M + Na]⁺ |
| A249 | 2-fluorophenyl | — | H | H | 2-Br | 3.004 min; m/z = 350 [M + H]⁺ |
| A250 | 2-fluorophenyl | — | nC₄H₉ | H | 2-Cl | 3.876 min; m/z = 361 [M + H]⁺ |
| A251 | 2-fluorophenyl | — | 4- | H | 2,4-Cl₂ | 4.466 min; m/z = 385 [M + Na]⁺ |
| | | | chlorobenzyl | | | |
| A252 | 2-fluorophenyl | — | 4- | H | 4-F | 3.981 min; m/z = 413 [M + H]⁺ |
| | | | chlorobenzyl | | | |
| A253 | 2-fluorophenyl | — | phenyl | H | 2-Cl | 3.677 min; m/z = 381 [M + H]⁺ |
| A254 | 2-fluorophenyl | — | H | H | 3-Br | 3.064 min; m/z = 372 [M + Na]⁺ |
| A255 | 2-fluorophenyl | — | OC₂H₅ | H | 2-Cl-5-NO₂ | 3.569 min; m/z = 394 [M + H]⁺ |
| A256 | 2-fluorophenyl | — | H | H | 2-Cl-5-NO₂ | 3.027 min; m/z = 350 [M + H]⁺ |
| A257 | 2-fluorophenyl | — | CH₃ | H | 2-Cl-5-NO₂ | 3.242 min; m/z = 364 [M + H]⁺ |
| A258 | 2-fluorophenyl | — | CH₃ | H | 2,5-F₂ | 3.112 min; m/z = 321 [M + H]⁺ |
| A259 | 2-fluorophenyl | — | CH₃ | H | 2,3-F₂ | 3.127 min; m/z = 321 [M + H]⁺ |
| A260 | 2-fluorophenyl | — | CH₃ | H | 3,5-F₂ | 3.206 min; m/z = 321 [M + H]⁺ |
| A261 | 2-fluorophenyl | — | CH₃ | H | 3,4-F₂ | 3.142 min; m/z = 321 [M + H]⁺ |
| A262 | 2-fluorophenyl | — | C₂H₅ | H | 2-F | 3.257 min; m/z = 317 [M + H]⁺ |
| A263 | 2-fluorophenyl | — | C₂H₅ | H | 2,3-F₂ | 3.359 min; m/z = 335 [M + H]⁺ |
| A264 | 2-fluorophenyl | — | C₂H₅ | H | 3,4-F₂ | 3.400 min; m/z = 335 [M + H]⁺ |
| A265 | 2-fluorophenyl | — | C₂H₅ | H | 3,5-F₂ | 3.452 min; m/z = 335 [M + H]⁺ |
| A266 | 2-fluorophenyl | — | CH₃ | H | 2-OCH₃ | 2.873 min; m/z = 337 [M + Na]⁺ |
| A267 | 2-fluorophenyl | — | CH₃ | H | 4-F | 3.007 min; m/z = 303 [M + H]⁺ |
| A268 | 2-fluorophenyl | — | CH₃ | H | 3-CH₃ | 3.144 min; m/z = 299 [M + H]⁺ |
| A269 | 2-fluorophenyl | — | C₂H₅ | H | 4-F | 3.274 min; m/z = 317 [M + H]⁺ |
| A270 | 2-fluorophenyl | — | CH₃ | H | 3-OCH₃ | 2.901 min; m/z = 315 [M + H]⁺ |
| A271 | 2-fluorophenyl | — | CH₃ | H | 2-F | 3.005 min; m/z = 303 [M + H]⁺ |
| A272 | 2-fluorophenyl | — | CH₃ | H | 3-CN-4-F | 2.932 min; m/z = 328 [M + H]⁺ |
| A273 | 2-fluorophenyl | — | CH₃ | H | 2-Cl | 3.192 min; m/z = 319 [M + H]⁺ |
| A274 | 2-fluorophenyl | — | CH₃ | H | 3-OCH₃-4-F | 2.958 min; m/z = 355 [M + Na]⁺ |
| A275 | 2-fluorophenyl | — | H | H | 2-CF₃ | 3.088 min; m/z = 339 [M + H]⁺ |
| A276 | 2-fluorophenyl | — | C₂H₅ | CH₃ | — | 3.623 min; m/z = 313 [M + H]⁺ |
| A277 | CH₂C(O)OCH₂CH₃ | | CH₃ | H | — | 2.639 min; m/z = 277 [M + H]⁺ |
| A278 | CH₃ | | CH₃ | H | — | 2.054 min; m/z = 227 [M + Na]⁺ |
| A279 | CH₂CH₂CH₃ | | CH₃ | H | — | 2.244 min; m/z = 233 [M + H]⁺ |
| A280 | CH₂(CH₂)₂CH₂(OH) | | CH₃ | H | — | 2.135 min; m/z = 277 [M + H]⁺ |
| A281 | CH₂CH(OCH₃)₂ | | CH₃ | H | — | 2.356 min; m/z = 279 [M + H]⁺ |
| A282 | CH₂(CH₂)₂CH₃ | | CH₃ | H | — | 2.657 min; m/z = 247 [M + H]⁺ |
| A283 | CH₂(CH₂)₅CH₃ | | CH₃ | H | — | 3.344 min; m/z = 289 [M + H]⁺ |
| A284 | phenyl | S-CH₂ | CH₃ | CH₃ | 2-F | 3.709 min; m/z = 344 [M + H]⁺ |
| A285 | phenyl | S-CH₂ | CH₂CH₃ | CH₃ | — | 3.896 min; m/z = 341 [M + H]⁺ |
| A286 | phenyl | CH₂CH₂ | CH₂CH₃ | CH₃ | — | 3.851 min; m/z = 322 [M + H]⁺ |
| A287 | 2-furyl | — | Br | CH₃ | — | 3.161 min; m/z = 335 [M + H]⁺ |
| A288 | 5-isoxazolyl | — | Cl | CH₃ | — | 2.970 min; m/z = 292 [M + H]⁺ |
| A289 | 2-fluorophenyl | — | CH₃ | CH₃ | 4-F | 3.454 min; m/z = 316 [M + H]⁺ |
| A290 | 2-fluorophenyl | — | Cl | CH₃ | — | 3.357 min; m/z = 319 [M + H]⁺ |
| A291 | 2-fluorophenyl | — | Br | CH₃ | — | 3.409 min; m/z = 363 [M + H]⁺ |
| A292 | 2-fluorophenyl | — | CH₃ | CH₃ | 2-F | 3.418 min; m/z = 316 [M + H]⁺ |
| A293 | 2-fluorophenyl | — | CH₃ | CH₃ | 3-F | 3.447 min; m/z = 316 [M + H]⁺ |
| A294 | 2-fluorophenyl | — | H | CH₃ | — | 3.147 min; m/z = 284 [M + H]⁺ |
| A295 | 2-fluorophenyl | — | CH₃ | H | 3-OCH₂—O-4 | 2.847 min; m/z = 351 [M + Na]⁺ |
| A296 | 2-fluorophenyl | — | CH₃ | H | 2-CH₃ | 3.170 min; m/z = 321 [M + Na]⁺ |
| A297 | 2-fluorophenyl | — | H | H | 3-OCH₃, 4-OCOCH₃ | 2.616 min; m/z = 381 [M + Na]⁺ |
| A298 | 2-fluorophenyl | — | H | H | 2-CH₃,5-CH₃ | 3.133 min; m/z = 321 |
| | | | | | | [M + Na]⁺ |
| A299 | 2-fluorophenyl | — | H | H | 4-OC(CH₃)₃ | 3.159 min; m/z = 365 [M + Na]⁺ |
| A300 | 2-fluorophenyl | — | H | H | 4-CH₃ | 2.899 min; m/z = 307 [M + Na]⁺ |
| A301 | 2,4-difluorophenyl | — | H | H | — | 2.931 min; m/z = 325 [M + Na]⁺ |
| A302 | 2,4-difluorophenyl | — | CH₂CH₃ | H | — | 3.186 min; m/z = 339 [M + Na]⁺ |
| A303 | 2,4-difluorophenyl | — | CI | H | — | 3.088 min; m/z = 323 [M + H]⁺ |
| A304 | 2,4-difluorophenyl | — | Br | H | — | 3.139 min; m/z = 368 [M + H]⁺ |
| A305 | 2,4-difluorophenyl | — | CH₃ | H | 2-F | 3.007 min; m/z = 321 [M + H]⁺ |
| A306 | 2,4-difluorophenyl | — | CH₃ | H | 2-CH₃ | 3.125 min; m/z = 339 [M + Na]⁺ |
| A307 | 2,4-difluorophenyl | — | CH₃ | H | 2-OCH₃ | 2.889 min; m/z = 355 [M + Na]⁺ |
| A308 | 2,4-difluorophenyl | — | CH₃ | H | 3-F | 3.043 min; m/z = 321 [M + Na]⁺ |
| A309 | 2,4-difluorophenyl | — | CH₃ | H | 4-F | 3.011 min; m/z = 321 [M + H]⁺ |
| A310 | 2,4-difluorophenyl | — | CH₂CH₃ | H | 2,4-difluoro | 3.421 min; m/z = 353 [M + H]⁺ |
| A311 | 2,4-difluorophenyl | — | H | CH₃ | — | 3.263 min; m/z = 303 [M + H]⁺ |
| A312 | 2,4-difluorophenyl | — | CH₂CH₃ | CH₃ | — | 3.674 min; m/z = 331 [M + H]⁺ |
| A313 | 2,4-difluorophenyl | — | Cl | CH₃ | — | 3.422 min; m/z = 338 [M + H]⁺ |
| A314 | 2,4-difluorophenyl | — | Br | CH₃ | — | 3.464 min; m/z = 382 [M + H]⁺ |
| A315 | 2,4-difluorophenyl | — | CH₃ | CH₃ | 2-F | 3.490 min; m/z = 335 [M + H]⁺ |
| A316 | 2,4-difluorophenyl | — | CH₃ | CH₃ | 2-CH₃ | 3.669 min; m/z = 331 [M + H]⁺ |
| A317 | 2,4-difluorophenyl | — | CH₃ | CH₃ | 2-OCH₃ | 3.457 min; m/z = 347 [M + H]⁺ |
| A318 | 2,4-difluorophenyl | — | CH₃ | CH₃ | 3-F | 3.517 min; m/z = 335 [M + H]⁺ |
| A319 | 2,4-difluorophenyl | — | CH₃ | CH₃ | 3,5-difluoro | 3.606 min; m/z = 353 [M + H]⁺ |
| A320 | 2,4-difluorophenyl | — | CH₃ | CH₃ | 4-F | 3.523 min; m/z = 335 [M + H]⁺ |
| A321 | 2,4-difluorophenyl | — | CH₃ | CH₃ | — | 3.480 min; m/z = 317 [M + H]⁺ |

TABLE C

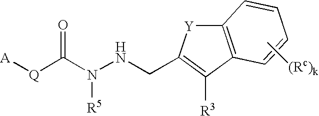

| No. | A | Q | R³ | R⁵ | (R^{c})ₖ | Y | m.p. or RT (HPLC/MS) |
|---|---|---|---|---|---|---|---|
| B1 | 3-pyridyl | — | H | H | — | O | 1.909 min; m/z = 268 [M + H]⁺ |
| B2 | 3-methylphenyl | — | H | H | — | O | 2.978 min; m/z = 281 [M + H]⁺ |
| B3 | 4-pyridyl | — | H | H | — | O | 1.878 min; m/z = 268 [M + H]⁺ |
| B4 | 4-methylphenyl | — | H | H | — | O | 2.961 min; m/z = 281 [M + H]⁺ |
| B5 | phenyl | CH₂ | H | H | — | O | 2.812 min; m/z = 281 [M + H]⁺ |
| B6 | 2-methylphenyl | — | H | H | — | O | 2.863 min; m/z = 281 [M + H]⁺ |
| B7 | 4-phenylphenyl | — | H | H | — | O | 3.477 min; m/z = 343 [M + H]⁺ |
| B8 | 3-chlorophenyl | — | H | H | — | O | 3.131 min; m/z = 323 [M + Na]⁺ |
| B9 | 2-naphthyl | — | H | H | — | O | 3.241 min; m/z = 317 [M + H]⁺ |
| B10 | 4-trifluoromethylphenyl | — | H | H | — | O | 3.322 min; m/z = 357 [M + Na]⁺ |
| B11 | 3-methoxyphenyl | — | H | H | — | O | 2.846 min; m/z = 297 [M + H]⁺ |
| B12 | 4-chlorophenyl | — | H | H | — | O | 3.120 min; m/z = 301 [M + H]⁺ |
| B13 | 4-nitrophenyl | — | H | H | — | O | 2.937 min; m/z = 334 [M + Na]⁺ |
| B14 | 2-chlorophenyl | — | H | H | — | O | 2.879 min; m/z = 301 [M + H]⁺ |
| B15 | 2-methoxyphenyl | — | H | H | — | O | 2.918 min; m/z = 297 [M + H]⁺ |
| B16 | 3-nitrophenyl | — | H | H | — | O | 2.934 min; m/z = 334 [M + Na]⁺ |
| B17 | 3,4-dimethoxyphenyl | — | H | H | — | O | 2.639 min; m/z = 327 [M + H]⁺ |
| B18 | 4-methoxyphenyl | — | H | H | — | O | 2.777 min; m/z = 297 [M + H]⁺ |
| B19 | 2,4-dichlorophenyl | — | H | H | — | O | 3.232 min; m/z = 357 [M + Na]⁺ |
| B20 | 2-chlorophenyl | OCH₂ | H | H | — | O | 3.221 min; m/z = 331 [M + H]⁺ |
| B21 | 1-(9H-xanthen-9-yl) | — | H | H | — | O | 3.443 min; m/z = 371 [M + H]⁺ |
| B22 | 2-thienyl | — | H | H | — | O | 2.723 min; m/z = 273 [M + H]⁺ |
| B23 | 2-benzothienyl | — | H | H | — | O | 3.298 min; m/z = 323 [M + H]⁺ |
| B24 | 2-phenoxy-3-pyridyl | — | H | H | — | O | 3.195 min; m/z = 360 [M + H]⁺ |
| B25 | 2-pyridyl | — | H | H | — | O | 2.585 min; m/z = 268 [M + H]⁺ |
| B26 | 3-trifluoromethylphenyl | — | H | H | — | O | 3.315 min; m/z = 357 [M + NA]⁺ |
| B27 | 4-hydroxyphenyl | — | H | H | — | O | 2.338 min; m/z = 283 [M + H]⁺ |
| B28 | phenyl | — | H | H | — | O | 2.750 min; m/z = 267 [M + H]⁺ |
| B29 | 4-dimethylaminophenyl | — | H | H | — | O | 2.564 min; m/z = 310 [M + H]⁺ |
| B30 | benzo-[1,3]-dioxol-5-yl | — | H | H | — | O | 2.750 min; m/z = 311 [M + H]⁺ |
| B31 | 4-fluorophenyl | — | H | H | — | O | 2.873 min; m/z = 285 [M + H]⁺ |
| B32 | 2-fluorophenyl | — | H | H | — | O | 2.852 min; m/z = 285 [M + H]⁺ |
| B33 | 3-fluorophenyl | — | H | H | — | O | 2.913 min; m/z = 285 [M + H]⁺ |
| B34 | phenyl | — | H | CH₃ | — | O | 3.048 min; m/z = 281 [M + H]⁺ |
| B35 | 2-furyl | — | H | CH₃ | — | O | 2.819 min; m/z = 271 [M + H]⁺ |
| B36 | 2-nitrophenyl | — | H | H | — | O | 2.825 min; m/z = 334 [M + Na]⁺ |
| B37 | 1-naphthyl | CH₂ | H | H | — | O | 3.292 min; m/z = 353 [M + Na]⁺ |
| B38 | 1-naphthyl | — | H | H | — | O | 3.214 min; m/z = 339 [M + Na]⁺ |
| B39 | 2,4-dichlorophenyl | OCH₂ | H | H | — | O | 3.586 min; m/z = 387 [M + NA]⁺ |
| B40 | 4-nitrophenyl | OCH₂ | H | H | — | O | 3.011 min; m/z = 364 [M + Na]⁺ |
| B41 | 2,4-dichlorophenyl | OCH(CH₃) | H | H | — | O | 3.706 min; m/z = 401 [M + Na]⁺ |
| B42 | 3-indolyl | CH₂ | H | H | — | O | 2.847 min; m/z = 320 [M + H]⁺ |
| B43 | 2-benzimidazolyl | SCH₂ | H | H | — | O | 2.322 min; m/z = 353 [M + H]⁺ |
| B44 | 4-methyl-imidazol-5-yl | — | H | H | — | O | 1.907 min; m/z = 293 [M + H]⁺ |
| B45 | 2-hydroxyphenyl | — | H | H | — | O | 3.070 min; m/z = 305 [M + Na]⁺ |
| B46 | 3-hydroxyphenyl | — | H | H | — | O | 2.508 min; m/z = 305 [M + Na]⁺ |
| B47 | phenyl | SCH₂ | H | CH₃ | — | O | 3.405 min; m/z = 327 [M + H]⁺ |
| B48 | phenyl | CH₂CH₂ | H | CH₃ | — | O | 3.379 min; m/z = 309 [M + H]⁺ |
| B49 | 1-methyl-1H-pyridin-2-on-3-yl | — | H | CH₃ | — | O | 2.244 min; m/z = 312 [M + H]⁺ |
| B50 | 2-fluorophenyl | — | H | H | 4-Cl,5-NO₂ | S | 3.301 min; m/z = 380 [M + H]⁺ |
| B51 | 2-fluorophenyl | — | H | H | — | S | 3.075 min; m/z = 301 [M + H]⁺ |
| B52 | 2-fluorophenyl | — | Cl | H | — | N | 3.079 min; m/z = 340 [M + Na]⁺ |
| B53 | 2-fluorophenyl | — | CH₃ | H | — | S | 3.254 min; m/z = 337 [M + Na]⁺ |
| B54 | 2-fluorophenyl | — | H | CH₃ | — | O | 3.049 min; m/z = 299 [M + H]⁺ |
| B55 | 2,4-difluorophenyl | — | H | H | — | O | 2.848 min; m/z = 303 [M + H]⁺ |
| B56 | 2,4-difluorophenyl | — | H | CH₃ | — | O | 3.129 min; m/z = 317 [M + H]⁺ |

The heteroatom of the moiety Q is attached to the radical A

TABLE D

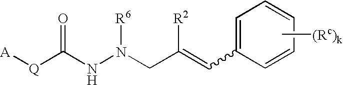

| No. | A | Q | R² | R⁶ | (R^{c})ₖ | RT (HPLC/MS) |
|---|---|---|---|---|---|---|
| C1 | 2-fluorophenyl | — | CH₃ | CH₃ | 3-F | 3.234 min; m/z = 317 [M + H]⁺ |
| C2 | 2-pyridyl | — | CH₃ | CH₃ | — | 2.882 min; m/z = 282 [M + H]⁺ |

TABLE E

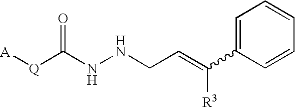

| No. | A | Q | R³ | RT (HPLC/MS) |
|---|---|---|---|---|
| D1 | CH₃ | | Cl | 2.060 min; m/z = 225 [M + H]⁺ |
| D2 | 2-fluorophenyl | — | Cl | 2.993 min; m/z = 327 [M + Na]⁺ |

TABLE F

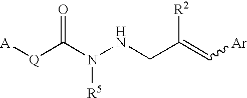

| No. | A | Q | R² | R⁵ | Ar | RT (HPLC/MS) and/or m/z |
|---|---|---|---|---|---|---|
| E1 | 2-pyridyl | — | H | H | 5-chloro-2-thienyl | 2.817 min; m/z = 316 [M + Na]⁺ |
| E2 | 2-pyridyl | — | H | H | 4-bromo-2-thienyl | 2.825 min; m/z = 360 [M + NA]⁺ |
| E3 | 2-pyridyl | — | CH(CH₃)₂ | H | 2-furyl | m/z = 308 [M + Na]⁺ |
| E4 | 2-pyridyl | — | CH₃ | H | 2-furyl | 2.471 min; m/z = 280 [M + Na]⁺ |
| E5 | 2-pyridyl | — | CH₃ | H | 3-pyridyl | 1.359 min; m/z = 269 [M + H]⁺ |
| E6 | 2-pyridyl | — | CH(CH₃)₂ | H | 3-pyridyl | m/z = 297 [M + H]⁺ |
| E7 | 2-fluorophenyl | — | H | H | 5-chloro-2-thienyl | 3.030 min; m/z = 333 [M + Na]⁺ |
| E8 | 2-fluorophenyl | — | H | H | 4-bromo-2-thienyl | 3.056 min; m/z = 377 [M + Na]⁺ |
| E9 | 2-fluorophenyl | — | CH₃ | H | 3-pyridyl | 1.526 min; m/z = 286 [M + H]⁺ |
| E10 | 2-fluorophenyl | — | H | H | 3-methyl-2-thienyl | 2.746 min; m/z = 313 [M + Na]⁺ |
| E11 | 2-fluorophenyl | — | CH(CH₃)₂ | H | 3-pyridyl | m/z = 314 [M + H]⁺ |
| E12 | CH₃ | | H | H | 5-chloro-2-thienyl | 1:992 min; m/z = 253 [M + Na]⁺ |
| E13 | CH₃ | | H | H | 4-bromo-2-thienyl | 2.022 min; m/z = 299 [M + Na]⁺ |
| E14 | CH₃ | | CH(CH₃)₂ | H | 2-furyl | 2.003 min; m/z = 223 [M + H]⁺ |
| | | | | | | 2.209 min; m/z = 245 [M + Na]⁺ |
| E15 | CH₃ | | CH₃ | H | 2-furyl | 1,589 min; m/z = 217 [M + Na]⁺ |
| E16 | CH₃ | | CH₃ | H | 3-pyridyl | m/z = 206 [M + H]⁺ |
| E17 | CH₃ | | H | H | 3-methyl-2-thienyl | 1,744 min; m/z = 233 [M + Na]⁺ |
| E18 | CH₃ | | CH(CH₃)₂ | H | 3-pyridyl | m/z = 234 [M + H]⁺ |
| E19 | CH₃ | | phenyl | H | 4-pyridyl | 1.422 min; m/z = 269 [M + H]⁺ |
| E20 | CH₃ | | CH₂CH₂CH₃ | H | 3-pyridyl | 1.306 min; m/z = 234 [M + H]⁺ |
| E21 | CH₃ | | CH₂CH₃ | H | 3-pyridyl | 1.051 min; m/z = 220 [M + H]⁺ |
| E22 | CH₃ | | phenyl | H | 2-furyl | 2.418 min; m/z = 279 [M + Na]⁺ |
| E23 | 2-pyridyl | — | phenyl | H | 4-pyridyl | 1.755 min; m/z = 331 [M + H]⁺ |
| E24 | 2-pyridyl | — | CH₂CH₂CH₃ | H | 3-pyridyl | 1.656 min; m/z = 297 [M + H]⁺ |
| E25 | 2-pyridyl | — | CH₂CH₃ | H | 3-pyridyl | 1.466 min; m/z = 283 [M + H]⁺ |
| E26 | 2-pyridyl | — | H | H | 5-nitro-furan-2-yl | 2.103 min; m/z = 289 [M + H]⁺ |
| E27 | 2-pyridyl | — | phenyl | H | 2-furyl | 2.983 min; m/z = 320 [M + H]⁺ |
| E28 | 2-fluorophenyl | — | phenyl | H | 4-pyridyl | 1.943 min; m/z = 348 [M + H]⁺ |
| E29 | 2-fluorophenyl | — | H | H | 2-furyl | 2.271 min; m/z = 283 [M + Na]⁺ |
| E30 | 2-fluorophenyl | — | CH₂CH₂CH₃ | H | 3-pyridyl | 1.835 min; m/z = 314 [M + H]⁺ |
| E31 | 2-fluorophenyl | — | CH₂CH₃ | H | 3-pyridyl | 1.630 min; m/z = 300 [M + H]⁺ |
| E32 | 2-fluorophenyl | — | phenyl | H | 2-furyl | 3.217 min; m/z = 359 [M + Na]⁺ |
| E33 | 2-fluorophenyl | — | CH₃ | H | 2-furyl | 2.713 min; m/z = 297 [M + Na]⁺ |
| E34 | 2-fluorophenyl | — | CH(CH₃)₂ | H | 2-furyl | m/z = 325 [M + Na]⁺ |
| E35 | CH₃ | | H | H | 2-furyl | 1.432 min; m/z = 203 [M + Na]⁺ |
| E36 | 2-pyridyl | — | H | H | 2-furyl | 2.100 min; m/z = 266 [M + Na]⁺ |
| E37 | 2-fluorophenyl | — | H | H | 5-nitro-furan-2-yl | 2.479 min; m/z = 328 [M + Na]⁺ |
| E38 | 2-pyridyl | — | H | H | 3-methyl-2-thienyl | 2.442 min; m/z = 296 [M + Na]⁺ |

TABLE G

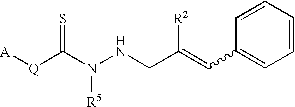

| No. | A | Q | R² | R⁵ | RT (HPLC/MS) |
|---|---|---|---|---|---|
| F1 | 2- | — | CH₃ | CH₃ | 3.952 min; m/z = 315 [M + H]⁺ |
| | fluorophenyl | | | | |

TABLE H

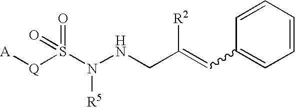

| No. | A | Q | R² | R⁵ | RT (HPLC/MS) |
|---|---|---|---|---|---|
| G1 | 4-methyl- | — | CH₃ | H | 3.543 min; m/z = 339 [M + Na]⁺ |
| | phenyl | | | | |
| G2 | 4-methoxy- | — | CH₃ | H | 3.377 min; m/z = 355 [M + Na]⁺ |
| | phenyl | | | | |

| | | | | |
|---|---|---|---|---|
| G3 | CH₃ | CH₃ | H | 2.680 min; m/z = 263 [M + HNa]⁺ |

TABLE J

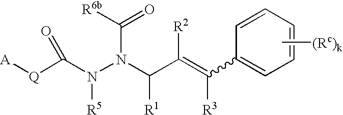

| No. | A | Q | R¹ | R² | R³ | R⁵ | R^{6b} | (R^{c})ₖ | RT (HPLC/MS) |
|---|---|---|---|---|---|---|---|---|---|
| H1 | 2-fluorophenyl | — | H | CH₃ | H | CH₃ | phenyl | — | 3.372 min; m/z = 402 [M + H]⁺ |
| H2 | 2-fluorophenyl | — | H | CH₃ | H | CH₃ | CH₂CH₂CH₃ | — | 3.156 min; m/z = 354 [M + H]⁺ |
| H3 | 2-fluorophenyl | — | H | CH₃ | H | CH₃ | OCH₂CH₂CH₃ | — | 3.521 min; m/z = 385 [M + H]⁺ |
| H4 | 2-fluorophenyl | — | H | CH₃ | H | CH₃ | phenyl | 3-F | 3.389 min; m/z = 420 [M + H]⁺ |
| H5 | 2-fluorophenyl | — | H | CH₃ | H | CH₃ | CH₂CH₂CH₃ | 3-F | 3.185 min; m/z = 372 [M + H]⁺ |
| H6 | 2-fluorophenyl | — | H | CH₃ | H | CH₃ | 5-isoxazolyl | — | 3.371 min; m/z = 393 [M + H]⁺ |
| H7 | 2-fluorophenyl | — | H | CH₃ | H | CH₃ | 2-furyl | — | 3.422 min; m/z = 392 [M + H]⁺ |
| H8 | 2-fluorophenyl | — | H | CH₃ | H | CH₃ | methoxycarbonyl | — | 3.419 min; m/z = 384 [M + H]⁺ |
| H9 | 2-fluorophenyl | — | H | CH₃ | H | CH₃ | cyclopropyl | — | 3.392 min; m/z = 366 [M + H]⁺ |
| H10 | 2-fluorophenyl | — | H | CH₃ | H | CH₃ | phenoxymethyl | — | 3.703 min; m/z = 433 [M + H]⁺ |
| H11 | 2-fluorophenyl | — | H | CH₃ | H | CH₃ | 2-methyl sulfanyl- | — | 3.502 min; m/z = 401 [M + Na]⁺ |
| | | | | | | | ethyl | | |
| H12 | 2-fluorophenyl | — | H | CH₃ | H | CH₃ | CH═CHCH₃ | — | 3.411 min; m/z = 366 [M + H]⁺ |
| H13 | 2-fluorophenyl | — | H | CH₃ | H | CH₃ | 2-thienyl | — | 3.595 min; m/z = 409 [M + H]⁺ |
| H14 | 2-fluorophenyl | — | H | CH₃ | H | CH₃ | 2,2,2-trifluorethoxy | — | 3.773 min; m/z = 424 [M + H]⁺ |
| H15 | 2-fluorophenyl | — | H | CH₃ | H | CH₃ | cyclopropyl | 3-F | 3.662 min; m/z = 384 [M + H]⁺ |
| H16 | 2-fluorophenyl | — | | CH₃ | | CH₃ | phenoxymethyl | 3-F | 3.971 min; m/z = 450 [M + H]⁺ |
| H17 | 2-fluorophenyl | — | H | CH₃ | H | CH₃ | 2-methyl sulfanyl- | 3-F | 3.774 min; m/z = 419 [M + H]⁺ |
| | | | | | | | ethyl | | |
| H18 | 2-fluorophenyl | — | H | CH₃ | H | CH₃ | CH═CHCH₃ | 3-F | 3.680 min; m/z = 384 [M + H]⁺ |
| H19 | 2-fluorophenyl | — | H | CH₃ | H | CH₃ | 2-thienyl | 3-F | 3.864 min; m/z = 427 [M + H]⁺ |
| H20 | 2-fluorophenyl | — | H | CH₃ | H | CH₃ | 2,2,2-trifluorethoxy | 3-F | 4.031 min; m/z = 442 [M + H]⁺ |
| H21 | 2-fluorophenyl | — | H | CH₃ | H | CH₃ | benzyl | — | 3.710 min; m/z = 417 [M + H]⁺ |
| H22 | 2-fluorophenyl | — | H | CH₃ | H | CH₃ | 1-naphthyl | — | 3.909 min; m/z = 452 [M + H]⁺ |
| H23 | 2-fluorophenyl | — | H | CH₃ | H | CH₃ | phenylsulfanyl- | — | 3.846 min; m/z = 449 [M + H]⁺ |
| | | | | | | | methyl | | |
| H24 | 2-fluorophenyl | — | H | CH₃ | H | CH₃ | 3-isoxazolyl | — | 3.469 min; m/z = 393 [M + Na]⁺ |
| H25 | 2-fluorophenyl | — | H | CH₃ | H | CH₃ | phenoxy | — | 3.862 min; m/z = 418 [M + H]⁺ |
| H26 | 2-fluorophenyl | — | H | CH₃ | H | CH₃ | 5-isoxazolyl | 3-F | 3.632 min; m/z = 411 [M + H]⁺ |
| H27 | 2-fluorophenyl | — | H | CH₃ | H | CH₃ | benzyl | 3-F | 3.980 min; mfz = 434 [M + H]⁺ |
| H28 | 2-fluorophenyl | — | H | CH₃ | H | CH₃ | 2-furyl | 3-F | 3.684 min; m/z = 410 [M + Na]⁺ |
| H29 | 2-fluorophenyl | — | H | CH₃ | H | CH₃ | 1-naphthyl | 3-F | 4.170 min; m/z = 471 [M + H]⁺ |
| H30 | 2-fluorophenyl | — | H | CH₃ | H | CH₃ | methoxycarbonyl | 3-F | 3.683 min; m/z = 402 [M + H]⁺ |
| H31 | 2-fluorophenyl | — | H | CH₃ | H | CH₃ | 3-isoxazolyl | 3-F | 3.732 min; m/z = 411 [M + Na]⁺ |
| H32 | 2-fluorophenyl | — | H | CH₃ | H | CH₃ | benzylamino | — | 3.743 min; m/z = 431 [M + H]⁺ |
| H33 | 2-fluorophenyl | — | H | CH₃ | H | CH₃ | CH₂CH═CH₂ | — | 3.434 min; m/z = 381 [M + H]⁺ |
| H34 | 2-fluorophenyl | — | H | CH₃ | H | CH₃ | phenylamino | — | 3.811 min; m/z = 417 [M + H]⁺ |
| H35 | 2-fluorophenyl | — | H | CH₃ | H | CH₃ | cyclohexylamino | — | 3.870 min; m/z = 423 [M + H]⁺ |
| H36 | 2-fluorophenyl | — | H | CH₃ | H | CH₃ | 1-naphthylamino | — | 3.864 min; m/z = 468 [M + H]⁺ |
| H37 | 2-fluorophenyl | — | H | CH₃ | H | CH₃ | ethylamino | — | 3.233 min; m/z = 369 [M + H]⁺ |
| H38 | 2-fluorophenyl | — | H | CH₃ | H | CH₃ | benzoylamino | — | 3.512 min; m/z = 445 [M + H]⁺ |
| H39 | 2-fluorophenyl | — | H | CH₃ | H | H | phenylsulfanyl- | 3-F | 3.820 min; m/z = 453 [M + Na]⁺ |
| | | | | | | | methyl | | |
| H40 | 2-fluorophenyl | — | H | CH₃ | H | H | 2,2,2-trifluorethoxy | 3-F | 3.717 min; m/z = 428 [M + H]⁺ |
| H41 | 2-fluorophenyl | — | H | CH₃ | H | H | phenoxy | 3-F | 3.790 min; m/z = 453 [M + Na]⁺ |
| H42 | 2-fluorophenyl | — | H | CH₃ | H | H | OCH2CH2CH3 | 3-F | 3.667 min; m/z = 388 [M + H]⁺ |
| H43 | 2-fluorophenyl | — | H | CH₃ | H | H | 5-isoxazolyl | 3-F | 3.309 min; m/z = 397 [M + Na]⁺ |
| H44 | 2-fluorophenyl | — | H | CH₃ | H | H | benzyl | 3-F | 3.663 min; m/z = 420 [M + H]⁺ |
| H45 | 2-fluorophenyl | — | H | CH₃ | H | H | 2-furyl | 3-F | 3.323 min; m/z = 396 [M + Na]⁺ |
| H46 | 2-fluorophenyl | — | H | CH₃ | H | H | 1-naphthyl | 3-F | 3.816 min; m/z = 457 [M + H]⁺ |
| H47 | 2-fluorophenyl | — | H | CH₃ | H | H | methoxycarbonyl | 3-F | 3.329 min; m/z = 388 [M + Na]⁺ |
| H48 | 2-fluorophenyl | — | H | CH₃ | H | H | cyclopropyl | 3-F | 3.309 min; m/z = 370 [M + H]⁺ |
| H49 | 2-fluorophenyl | — | H | CH₃ | H | H | phenoxymethyl | 3-F | 3.693 min; m/z = 436 [M + H]⁺ |
| H50 | 2-fluorophenyl | — | H | CH₃ | H | H | phenyl | 3-F | 3.510 min; m/z = 406 [M + Na]⁺ |
| H51 | 2-fluorophenyl | — | H | CH₃ | H | H | 2-methyl sulfanyl- | 3-F | 3.453 min; m/z = 405 [M + Na]⁺ |
| | | | | | | | ethyl | | |
| H52 | 2-fluorophenyl | — | H | CH₃ | H | H | CH═CHCH₃ | 3-F | 3.329 min; m/z = 370 [M + H]⁺ |
| H53 | 2-fluorophenyl | — | H | CH₃ | H | H | 2-thienyl | 3-F | 3.516 min; m/z = 413 [M + Na]⁺ |
| H54 | 2-fluorophenyl | — | H | CH₃ | H | H | 3-isoxazolyl | 3-F | 3.335 min; m/z = 397 [M + Na]⁺ |
| H55 | 2-fluorophenyl | — | H | CH₃ | H | H | CH₂CH₃ | 3-F | 3.278 min; m/z = 358 [M + Na]⁺ |

TABLE K

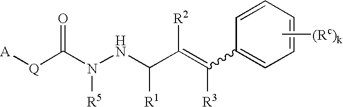

| No. | A | Q | R¹ | R² | R³ | R⁵ | (R^{c})ₖ | RT (HPLC/MS) |
|---|---|---|---|---|---|---|---|---|
| J1 | CH₃ | | H | F | F | H | 3-OCH₃ | 2.199 min; m/z = 256 [M + Na]⁺ |
| J2 | 2-fluorophenyl | — | H | F | F | H | 3-OCH₃ | 2.872 min; m/z = 336 [M + Na]⁺ |
| J3 | 2-furyl | — | H | F | F | H | 3-OCH₃ | 2.562 min; m/z = 308 [M + Na]⁺ |
| J4 | 2,4-difluorophenyl | — | H | F | F | H | 3-OCH₃ | 2.981 min; m/z = 354 [M + Na]⁺ |
| J5 | 2-fluorophenyl | — | H | F | F | CH₃ | 3-OCH₃ | 3.081 min; m/z = 350 [M + H]⁺ |
| J6 | 2,4-difluorophenyl | — | H | F | F | CH₃ | 3-OCH₃ | 3.101 min; m/z = 368 [M + H]⁺ |
| J7 | 2-fluorophenyl | — | 3-pyridyl | H | H | H | 4-F | 2.321 min; m/z = 365 [M + Na]⁺ |
| J8 | 2-pyridyl | — | CH₃ | H | H | H | 4-F | 2.585 min; m/z = 285 [M + Na]⁺ |
| J9 | CH₃ | | CH₃ | H | H | H | 4-F | 1.976 min; m/z = 222 [M + Na]⁺ |
| J10 | 2-furyl | — | cyclopropyl | H | H | H | 4-F | 2.871 min; m/z = 300 [M + Na]⁺ |
| J11 | 2-furyl | — | CH₃ | H | H | H | 4-F | 2.741 min; m/z = 274 [M + H]⁺ |
| J12 | 2-furyl | — | 3-pyridyl | H | H | H | 4-F | 2.166 min; m/z = 337 [M + Na]⁺ |
| J13 | 2-fluorophenyl | — | CH₃ | H | H | H | 4-F | 2.829 min; m/z = 302 [M + Na]⁺ |
| J14 | 2-fluorophenyl | — | phenyl | H | H | H | 4-F | 3.609 min; m/z = 364 [M + Na]⁺ |
| J15 | CH₃ | | 3-pyridyl | H | H | H | 4-F | 1.754 min; m/z = 285 [M + H]⁺ |
| J16 | 2-pyridyl | — | cyclopropyl | H | H | H | 4-F | 2.877 min; m/z = 311 [M + Na]⁺ |
| J17 | 2,4-difluorophenyl | — | CH₃ | H | H | H | 4-F | 2.938 min; m/z = 320 [M + Na]⁺ |
| J18 | 2,4-difluorophenyl | — | cyclopropyl | H | H | H | 4-F | 3.188 min; m/z = 346 [M + Na]⁺ |
| J19 | 2,4-difluorophenyl | — | phenyl | H | H | H | 4-F | 3.667 min; m/z = 382 [M + Na]⁺ |
| J20 | 2-furyl | — | phenyl | H | H | H | 4-F | 3.416 min; m/z = 336 [M + Na]⁺ |
| J21 | 2-furyl | — | CH₃ | H | H | CH₃ | 4-F | 3.186 min; m/z = 288 [M + Na]⁺ |
| J22 | 2-furyl | — | 3-pyridyl | H | H | CH₃ | 4-F | 2.339 min; m/z = 351 [M + Na]⁺ |
| J23 | 2,4-difluorophenyl | — | cyclopropyl | H | H | CH₃ | 4-F | 3.817 min; m/z = 360 [M + H]⁺ |
| J24 | 2,4-difluorophenyl | — | 3-pyridyl | H | H | CH₃ | 4-F | 2.425 min; m/z = 397 [M + H]⁺ |
| J25 | 2,4-difluorophenyl | — | phenyl | H | H | CH₃ | 4-F | 3.927 min; m/z = 396 [M + Na]⁺ |
| J26 | 2,4-difluorophenyl | — | 3-pyridyl | H | H | H | 4-F | 2.403 min; m/z = 383 [M + H]⁺ |

TABLE L

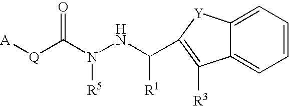

| No. | A | Q | R¹ | R³ | R⁵ | Y | RT (HPLC/MS) |
|---|---|---|---|---|---|---|---|
| K1 | 2-fluorophenyl | — | CH₃ | H | H | O | 3.047 min; m/z = 298 [M + Na]⁺ |
| K2 | 2-fluorophenyl | — | CH₃ | H | CH₃ | OCH₂ | 3.208 min; m/z = 326 [M + H]⁺ |
| K3 | 2-fluorophenyl | — | CH₃ | H | CH₃ | O | 3.137 min; m/z = 312 [M + H]⁺ |
| K4 | 2-fluorophenyl | — | CH₃ | H | H | OCH₂ | 2.914 min; m/z = 312 [M + Na]⁺ |
| K5 | 2-pyridyl | — | CH₃ | H | H | O | 2.710 min; m/z = 281 [M + Na]⁺ |
| K6 | CH₃ | | CH₃ | H | H | O | 2.075 min; m/z = 218 [M + Na]⁺ |
| K7 | 2-furyl | — | CH₃ | H | H | O | 2.727 min; m/z = 270 [M + Na]⁺ |
| K8 | 2-furyl | — | CH₃ | H | CH₃ | O | 3.057 min; m/z = 284 [M + H]⁺ |
| K9 | 2,4-difluorophenyl | — | CH₃ | H | CH₃ | O | 3.307 min; m/z = 330 [M + H]⁺ |
| K10 | 2,4-difluorophenyl | — | CH₃ | H | H | O | 3.065 min; m/z = 316 [M + Na]⁺ |

The action of the compounds of the formula I against pests was demonstrated by the following experiments:

I Activity Against Insects

I.1 Cotton Aphid (aphis gossypil)

The active compounds were formulated in 50:50 acetone:water and 100 ppm Kinetics surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, compounds A10, A11, A16, A19, A21, A44, A47, A63, A68, B11, B20 and B25 at 300 ppm showed over 75% mortality in comparison with untreated controls.

I.2 Green Peach Aphid (Myzus persicae)

The active compounds were formulated in 50:50 acetone:water and 100 ppm Kinetic® surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, compounds compounds A13, A51, A70, A74, A75, A79, A80, A81, A83, B1, B22 and B34 at 300 ppm showed over 75% mortality in comparison with untreated controls.

I.3 Orchid thrips (dichromothrips corbetti)

Dichromothrips corbetti adults used for bioassay were obtained from a colony maintained continuously under laboratory conditions. For testing purposes, the test compound was diluted to a concentration of 500 ppm (wt compound: vol diluent) in a 1:1 mixture of acetone:water, plus 0.01% Kinetic® surfactant.

Thrips potency of each compound was evaluated by using a floral-immersion technique. Plastic petri dishes were used as test arenas. All petals of individual, intact orchid flowers were dipped into treatment solution for approximately 3 seconds and allowed to dry for 2 hours. Treated flowers were placed into individual petri dishes along with 10-15 adult thrips. The petri dishes were then covered with lids. All test arenas were held under continuous light and a temperature of about 28° C. for duration of the assay. After 4 days, the numbers of live thrips were counted on each flower, and along inner walls of each petri dish. The level of thrips mortality was extrapolated from pre-treatment thrips numbers.

In this test, compounds A1, A2, A12, A141, A149, A 155, A173, A218, A223, A260, A301, A303, A305, A319, C1, E3 and E42 at 300 ppm showed over 75% mortality in comparison with untreated controls.

I.4 Bean Aphid (Aphis fabae)

Nasturtium plants grown in Metro mix in the ₁st leaf-pair stage (variety ‘Mixed Jewel’) were infested with approximately 2-30 laboratory-reared aphids by placing infested cut plants on top of the test plants. The cut plants were removed after 24 hr. Each plant was dipped into the test solution to provide complete coverage of the foliage, stem, protruding seed surface and surrounding cube surface and allowed to dry in the fume hood. The treated plants were kept at about 25° C. with continuous fluorescent light. Aphid mortality is determined after 3 days.

In this test, compounds A93, A141, A305, E1, E3 and K6 at 300 ppm showed 75% mortality in comparison with untreated controls.

1-42. (canceled)

43. A method for combating pests, selected from insects, arachnids and nematodes, which comprises contacting said pests, their habitat, breeding ground, food supply, plant, seed, soil, area, material or environment in which the animal pests are growing or may grow, or the materials, plants, seeds, soils, surfaces or spaces to be protected from an attack of or infestation by said pest, with a pesticidally effective amount of a hydrazide compound of general formula I 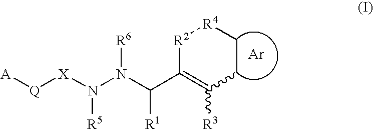
wherein
----- is absent or a covalent bond;
A is a cyclic radical selected from phenyl and a 5- or 6-membered heterocyclic radical with I to 4 heteroatoms as ring members which are selected, independently of one another, from O, N and S, the 5- or 6-membered heterocyclic radical may have a carbonyl group as ring member and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄-alkylidene, S—C₁-C₄-alkylidene and NR⁹—C₁-C₄-alkylidene, wherein the alkylidene group in the last four mentioned radicals is unsubstituted or carries 1, 2, 3 or 4 substituents selected from OH, ═O, halogen, C₁-C₄-haloalkyl and C₁-C₄-alkoxy; or
A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above, and wherein two radicals R^{c} which are bound to adjacent carbon atoms may form a O—CH₂—O moiety, where in said moiety 1 or 2 hydrogen atoms may be replaced by a radical R^{b} as defined above;
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, phenyl and a 5- or 6-membered heterocyclic radical with 1 to 4 heteroatoms which are selected, independently of one another, from O, N and S, it being possible for the aforementioned two last radicals to be unsubstituted or substituted by 1, 2, 3, 4 or 5 substituents R^{a} as defined above;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
R⁴ together with R² is a bivalent radical, which is selected from O, S, CR¹⁰═N, N═N, O—CR¹¹R¹², S—CR¹¹R¹², N(R¹³)—CR¹¹R¹², O—C(O), O—C(S), S—(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkoxycarbonyl, C₂-C₁₀-alkenylcarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO—, benzoyl (C₆H₅—CO—), C₃-C₁₀-cycloalkylcarbonyl, R^{6a}—CO—, wherein R⁶a is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl, benzoyl, naphthyl or benzyl, wherein the phenyl ring in the aforementioned four radicals may be substituted by 1, 2, 3, 4 or 5 substituents R^{b}; and
R¹⁰is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};
or a salt thereof.

44. The method as claimed in claim 43, wherein the compounds of general formula I are selected from compounds or a salt thereof, wherein Ar in formula I is phenyl, which is unsubstituted or substituted by 1, 2, 3 or 4 radicals R^{c}.

45. The method as claimed in claim 43, wherein the compounds of general formula I are selected from compounds or a salt thereof, wherein X in formula I is C═O.

46. The method as claimed in claim 43, wherein the compounds of general formula I are selected from compounds or a salt thereof, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be substituted by 1, 2 or 3 substituents R^{a}.

47. The method as claimed in claim 43, wherein the compounds of general formula I are selected from compounds or a salt thereof, wherein A in formula I is a cyclic radical selected from phenyl, thienyl, and pyridyl, where the cyclic radical may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

48. The method as claimed in claim 43, wherein the compounds of general formula I are selected from compounds or a salt thereof, wherein A in formula I is 2-thienyl which may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

49. The method as claimed in claim 43, wherein the compounds of general formula I are selected from compounds or a salt thereof, wherein Q in formula I is selected from a single bond, —CH₂—, O—CH₂—, —S—CH₂—, —O—CH(CH₃)— and —S—CH(CH₃)—.

50. The method as claimed in claim 43, wherein the compounds of general formula I are selected from compounds or a salt thereof, wherein Q and A together are C₁-C₁₀-alkyl which may be substituted by 1 or 2 substituents selected from the group consisting of ═O, OH and and C₁-C₂-alkoxy.

51. The method as claimed in claim 43, wherein the compounds of general formula I are selected from compounds or a salt thereof, wherein R¹ is selected from the group consisting of hydrogen, C₁-C₁₀-alkyl and C₁-C₁₀-haloalkyl.

52. The method as claimed in claim 43, wherein the compounds of general formula I are selected from compounds or a salt thereof, wherein R² in formula I is selected from hydrogen, halogen and C₁-C₄-alkyl.

53. The method as claimed in claim 43, wherein the compounds of general formula I are selected from compounds or a salt thereof, wherein R³ in formula I is selected from hydrogen, halogen and C₁-C₄-alkyl.

54. The method as claimed in claim 43, wherein the compounds of general formula I are selected from compounds or a salt thereof, wherein R³ in formula I is hydrogen.

55. The method as claimed in claim 43, wherein the compounds of general formula I are selected from compounds or a salt thereof, wherein R⁴ is hydrogen or a radical R^{c}.

56. The method as claimed in claim 55, wherein the compounds of general formula I are selected from compounds or a salt thereof, wherein Ar is phenyl, which is unsubstituted or substituted by 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

57. The method as claimed in claim 43, wherein the compounds of general formula I are selected from compounds or a salt thereof, wherein R⁴ together with R² is —O—.

58. The method as claimed in claim 57, wherein the compounds of general formula I are selected from compounds or a salt thereof, wherein Ar in formula I is phenyl, which is unsubstituted or which may carry 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

59. The method as claimed in claim 43, wherein the compounds of general formula I are selected from compounds or a salt thereof, wherein R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl.

60. The method as claimed in claim 43, wherein the compounds of general formula I are selected from compounds or a salt thereof, wherein R⁶ is hydrogen.

61. The method as claimed in claim 43, wherein the pests are insects.

62. A method for protecting crops from attack or infestation by pests, selected from insects, arachnids and nematodes, the method comprising contacting a crop with a pesticidally effective amount of at least one compound of formula I 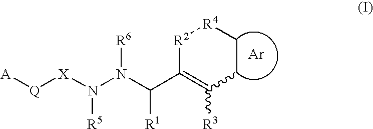
wherein
----- is absent or a covalent bond;
A is a-cyclic radical selected from phenyl and a 5- or 6-membered heterocyclic radical with 1 to 4 heteroatoms as ring members which are selected, independently of one another, from O, N and S, the 5- or 6-membered heterocyclic radical may have a carbonyl group as ring member and where the cyclic radical may have 1, 2, 3, 4 or 5 substituents R^{a} which are selected, independently of one another, from halogen, cyano, nitro, C₁-C₁₀-alkyl, C₁C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁l-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄-alkylidene, S—C₁-C₄-alkylidene and NR⁹-C₁-C₄-alkylidene, wherein the alkylidene group in the last four mentioned radicals is unsubstituted or carries 1, 2, 3 or 4 substituents selected from OH, ═O, halogen, C₁-C₄-haloalkyl and C₁-C₄-alkoxy; or
A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above, and wherein two radicals R^{c} which are bound to adjacent carbon atoms may form a O—CH₂—O moiety, where in said moiety 1 or 2 hydrogen atoms may be replaced by a radical R^{b} as defined above;
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, phenyl and a 5- or 6-membered heterocyclic radical with 1 to 4 heteroatoms which are selected, independently of one another, from O, N and S, it being possible for the aforementioned two last radicals to be unsubstituted or substituted by 1, 2, 3, 4 or 5 substituents R^{a} as defined above;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
R⁴ together with R² is a bivalent radical, which is selected from O, S, CR¹⁰═N, N═N, O—CR¹¹R¹², S—CR¹¹R¹², N(R¹³)—CR¹¹R¹², O—C(O), O—C(S), S—C(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkoxycarbonyl, C₂-C₁₀-alkenylcarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO—, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl, benzoyl, naphthyl or benzyl, wherein the phenyl ring in the aforementioned four radicals may be substituted by 1, 2, 3, 4 or 5 substituents R^{b}; and
R¹⁰ is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};
or a salt thereof.

63. A method for protecting non-living materials from attack or infestation by pests, selected from insects, arachnids and nematodes, the method comprising contacting the non-living material with a pesticidally effective amount of at least one compound of formula I 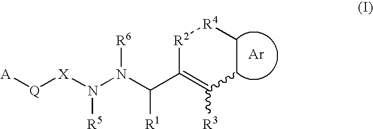
wherein
----- is absent or a covalent bond;
A is a cyclic radical selected from phenyl and a 5- or 6-membered heterocyclic radical with 1 to 4 heteroatoms as ring members which are selected, independently of one another, from O, N and S, the 5- or 6-membered heterocyclic radical may have a carbonyl group as ring member and where the cyclic radical may have 1, 2, 3, 4 or 5 substituents R^{a} which are selected, independently of one another, from halogen, cyano, nitro, C₁-C₁₀-alkyl, C₁C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄-alkylidene, S—C₁-C₄-alkylidene and NR⁹—C₁-C₄-alkylidene, wherein the alkylidene group in the last four mentioned radicals is unsubstituted or carries 1, 2, 3 or 4 substituents selected from OH, ═O, halogen, C₁-C₄-haloalkyl and C₁-C₄-alkoxy; or
A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above, and wherein two radicals R^{c} which are bound to adjacent carbon atoms may form a O—CH₂—O moiety, where in said moiety 1 or 2 hydrogen atoms may be replaced by a radical R^{b} as defined above;
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, phenyl and a 5- or 6-membered heterocyclic radical with 1 to 4 heteroatoms which are selected, independently of one another, from O, N and S, it being possible for the aforementioned two last radicals to be unsubstituted or substituted by 1, 2, 3, 4 or 5 substituents R^{a} as defined above;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy- C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
R⁴ together with R² is a bivalent radical, which is selected from O, S, CR¹⁰═N, N═N, O—CR¹¹R¹², S—CR¹¹R¹², N(R¹³)—CR¹¹R¹², O—C(O), O—C(S), S—C(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkoxycarbonyl, C₂-C₁₀-alkenylcarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO—, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl R⁷R⁸N—CO—, phenyl, benzoyl, naphthyl or benzyl, wherein the phenyl ring in the aforementioned four radicals may be substituted by 1, 2, 3, 4 or 5 substituents R^{b}; and
R¹⁰ is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C ₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};
or a salt thereof.

64. A hydrazide compound of the general formula I 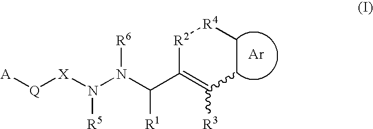
wherein
----- is absent or a covalent bond;
A is a cyclic radical selected from phenyl and a 5- or 6-membered heterocyclic radical with 1 to 4 heteroatoms as ring members which are selected, independently of one another, from O, N and S, the 5- or 6-membered heterocyclic radical may have a carbonyl group as ring member and where the cyclic radical may have 1, 2, 3, 4 or 5 substituents R^{a} which are selected, independently of one another, from halogen, cyano, nitro, C₁-C₁₀-alkyl, C₁C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄-alkylidene, S—C₁-C₄-alkylidene and NR⁹—C₁-C₄-alkylidene, wherein the alkylidene group in the last four mentioned radicals is unsubstituted or carries 1, 2, 3 or 4 substituents selected from OH, ═O, halogen, C₁-C₄-haloalkyl and C₁-C₄-alkoxy; or
A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above, and wherein two radicals R^{c} which are bound to adjacent carbon atoms may form a O—CH₂—O moiety, where in said moiety 1 or 2 hydrogen atoms may be replaced by a radical R^{b} as defined above;
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, phenyl and a 5- or 6-membered heterocyclic radical with 1 to 4 heteroatoms which are selected, independently of one another, from O, N and S, it being possible for the aforementioned two last radicals to be unsubstituted or substituted by 1, 2, 3, 4 or 5 substituents R^{a} as defined above;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
R⁴ together with R² is a bivalent radical, which is selected from O, S, CR¹⁰═N, N═N, O—CR¹¹R¹², S—CR¹¹R¹², N(R¹³)—CR¹¹R¹², O—C(O), O—C(S), S—C(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkoxycarbonyl, C₂-C₁₀-alkenylcarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO—, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl, benzoyl, naphthyl or benzyl, wherein the phenyl ring in the aforementioned four radicals may be substituted by 1, 2, 3, 4 or 5 substituents R^{b}; and
R¹⁰ is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};
or a salt thereof,
except for compounds of formula I, wherein
A and Ar are unsubstituted phenyl, Q is a single bond, X is C═O and R¹, R², R³, R⁴, R⁵ and R⁶ are hydrogen; or
A and Ar are unsubstituted phenyl, Q is a single bond, X is C═O, R¹ is CH₂—CH═CH₂ or C₆H₅ and R², R³, R⁴, R⁵ and R⁶ are hydrogen; or
Ar is unsubstituted phenyl, A together with Q is CH₃, X is C═O, R¹ is hydrogen, CH₃ or C₆H₅ and R², R³, R⁴, R⁵ and R⁶ are hydrogen; or
Ar is unsubstituted phenyl, A is 4-methylphenyl, Q is a single bond, X is SO₂, R¹ is (CH₂)₃CH₃ and R² is methyl, R³, R⁴, R⁵ and R⁶ are hydrogen; or
Ar is unsubstituted phenyl, A is 4-methylphenyl, Q is a single bond, X is SO₂; R¹ is C₆H₅ or CH₃ and R², R³, R⁴, R⁵ and R⁶ are hydrogen; or
A is 5-methyl-3-phenylisoxazol-4-yl, Ar is pyridin-2-yl, Q is a single bond, X is C═O, R¹ is methyl and R², R³, R⁴, R⁵ and R⁶ are hydrogen; or
Ar is phenyl, X is C═S, R¹ is methyl, R² together with R⁴ is CH═N, R³, R⁵ and R⁶ are hydrogen and A together with Q form a radical which is selected from the group consisting of NH(C₆H₅), N(CH₃)₂, N(CH₃)(cyclohexyl), piperidin-1-yl, 4-methylpiperidin-1-yl, 4-phenylpiperidin-1-yl, 4-ethoxycarbonylpiperazin-1-yl and 2,6-dimethylmorpholin-4-yl.

65. The compound as claimed in claim 64, wherein
A is a cyclic radical selected from phenyl and a 5- or 6-membered heterocyclic radical with 1 to 4 heteroatoms which are selected, independently of one another, from O, N and S, and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkyl-thio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b};
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be unsubstituted or substituted by 1, 2, 3, 4 or 5 substituents R^{b};
R¹ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₂-C₁₀-haloalkynyl;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO— and benzyl wherein benzyl may be substituted by 1, 2, 3, 4 or 5 substituents R^{b} as defined above; and
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl or benzyl wherein phenyl and benzyl may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl.

66. The compound as claimed in claim 64, wherein Ar in formula I is phenyl, which is unsubstituted or substituted by 1, 2, 3 or 4 radicals R^{c}.

67. The compound as claimed in claim 64, wherein X in formula I is C═O.

68. The compound as claimed in claim 64, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be substituted by 1, 2 or 3 substituents R^{a}.

69. The compound as claimed in claim 64, wherein A in formula I is a cyclic radical selected from phenyl, thienyl, and pyridyl, where the cyclic radical may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

70. The compound as claimed in claim 64, wherein A in formula I is 2-thienyl which may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

71. The compound as claimed in claim 64, wherein Q in formula I is selected from a single bond, —CH₂—, O—CH₂—, —S—CH₂—, 13 O—CH(CH₃)— and —S—CH(CH₃)—.

72. The compound as claimed in claim 64, wherein Q and A together are C₁-C₁₀-alkyl which may be substituted by 1 or 2 substituents selected from the group consisting of ═O, OH and C₁-C₂-alkoxy.

73. The compound of the general formula I as defined in claim 64 or a salt thereof, wherein R¹ is selected from the group consisting of hydrogen, C₁-C₁₀-alkyl and C₁-C₁₀-haloalkyl.

74. The compound as claimed in claim 64, wherein R² in formula I is selected from hydrogen, halogen and C₁-C₄-alkyl.

75. The compound as claimed in claim 64, wherein R³ in formula I is selected from hydrogen, halogen and C₁-C₄-alkyl.

76. The compound as claimed in claim 64, wherein R³ in formula I is hydrogen.

77. The compound as claimed in claim 64, wherein R⁴ is hydrogen or a radical R^{c}.

78. The compound as claimed in claim 77, wherein Ar is phenyl, which is unsubstituted or substituted by 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

79. The compound as claimed in claim 64, wherein R⁴ together with R² is —O—.

80. The compound as claimed in claim 79, wherein Ar in formula I is phenyl, which is unsubstituted or which may carry 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

81. The compound as claimed in claim 64, wherein R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl.

82. The compound as claimed in claim 64, wherein R⁶ is hydrogen.

83. A hydrazide compound of the general formula I 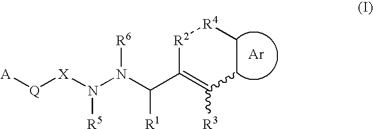
wherein
----- is absent or a covalent bond;
A is a cyclic radical selected from phenyl and a 5- or 6-membered heterocyclic radical with 1 to 4 heteroatoms as ring members which are selected, independently of one another, from O, N and S, the 5- or 6-membered heterocyclic radical may have a carbonyl group as ring member and where the cyclic radical may have 1, 2, 3, 4 or 5 substituents R^{a} which are selected, independently of one another, from halogen, cyano, nitro, C₁-C₁₀-alkyl, C₁C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein in each case two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Q is selected from the group consisting of a single bond, C₁-C₄ alkylidene, O—C₁-C₄-alkylidene, S—C₁-C₄-alkylidene and NR⁹—C₁-C₄-alkylidene, wherein the alkylidene group in the last four mentioned radicals is unsubstituted or carries 1, 2, 3 or 4 substituents selected from OH, ═O, halogen, C₁-C₄-haloalkyl and C₁-C₄-alkoxy; or
A-Q may together be C₁-C₁₀-alkyl, which may be substituted by 1 or 2 substituents selected from the group consisting of ═O, OH, C₁-C₄-alkoxy, C₁-C₄-alkylthio, halogen or C₁-C₄-alkylcarbonyloxy;
X is C═O, C═S or SO₂;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, C₁-C₁₀-alkylcarbonyloxy, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above, and wherein two radicals R^{c} which are bound to adjacent carbon atoms may form a O—CH₂—O moiety, where in said moiety 1 or 2 hydrogen atoms may be replaced by a radical R^{b} as defined above;
R¹ is hydrogen;
R² is a monovalent radical selected from H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R³ is selected from the group consisting of H, halogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl and phenyl which may be substituted by 1, 2, 3, 4 or 5 substituents R^{b} as defined above;
R⁴ is hydrogen or has one of the meanings given for R^{c} or
R⁴ together with R² is a bivalent radical, which is selected from O, S, CR¹⁰═N, N═N, O—CR¹¹R¹², S—CR¹¹R¹², N(R¹³)—CR¹¹R¹², O—C(O), O—C(S), S—C(O); N(R¹³)—C(O) or N(R¹³)—C(S);
R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, benzyl and phenyl wherein benzyl and phenyl may be substituted by 1, 2, 3, 4 or 5 substituents R^{d} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, where the alkyl moiety of alkylcarbonyl may be unsubstituted or substituted by 1, 2 or 3 substituents selected from the group consisting of C₁-C₁₀-alkylthio, phenylthio, phenyl and phenoxy, C₁-C₁₀-haloalkoxycarbonyl, C₂-C₁₀-alkenylcarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO—, benzoyl (C₆H₅—CO—), C₃-C₁₀-cycloalkylcarbonyl, R^{6a}—CO—, wherein R^{6a} is C₁-C₄-alkoxycarbonyl, phenoxy, naphthyl or a 5- or 6-membered heterocyclic radical with 1 to 4 heteroatoms which are selected, independently of one another, from O, N and S, it being possible for phenoxy, naphthyl and the 5- or 6-membered heterocyclic radical to be unsubstituted or substituted by 1, 2, 3, 4 or 5 substituents R^{a} as defined above, and benzyl wherein benzyl may be substituted by 1, 2, 3, 4 or 5 substituents R^{b} as defined above;
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl, benzoyl, naphthyl or benzyl wherein the phenyl ring in the aforementioned four radicals may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl;
R⁹ is hydrogen, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl, benzoyl, naphthyl or benzyl, wherein the phenyl ring in the aforementioned four radicals may be substituted by 1, 2, 3, 4 or 5 substituents R^{b}; and
R¹⁰ is hydrogen or has one of the meanings given for R^{c};
R¹¹, R¹² are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl and C₃-C₁₀-haloalkynyl, C₁-C₁₀-alkoxy, C₁-C₁₀-haloalkoxy, C₁-C₁₀-alkylthio, C₁-C₁₀-haloalkylthio, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl, one of the radicals R¹¹ or R¹² may also be C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl or R⁷R⁸N—CO—.
R¹³ is hydrogen, cyano, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-haloalkylsulfonyl, C₁-C₁₀-alkylcarbonyl, C₁-C₁₀-haloalkylcarbonyl, R⁷R⁸N—CO—, phenyl or benzyl, wherein phenyl and benzyl may be substituted by 1, 2, 3, 4 or 5 substituents R^{b};
or a salt thereof.

84. The compound as claimed in claim 83, wherein
A is a cyclic radical selected from phenyl and a 5- or 6-membered heterocyclic radical with 1 to 4 heteroatoms which are selected, independently of one another, from O, N and S, and where the cyclic radical may have 1, 2, 3, 4 or 5 substituents R^{a} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be substituted by 1, 2, 3, 4 or 5 substituents R^{b} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-haloalkenyloxy, C₂-C₁₀-alkynyloxy, C₃-C₁₀-haloalkynyloxy, C₁-C₁₀-alkylthio, C₁-C₁₀-haloalkyl-thio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl and R⁷R⁸N—CO—, wherein two radicals R^{a} which are bound to adjacent carbon atoms may form a CH═CH—CH═CH moiety or a O—CH₂—O moiety, where in these two moieties 1 or 2 hydrogen atoms may be replaced by a radical R^{b} as defined above;
Ar is an aromatic radical selected from phenyl, naphthyl, pyridyl, pyrimidyl, furyl and thienyl, where the aromatic radical may carry 1, 2, 3, 4 or 5 substituents R^{c} which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, C₁-C₁₀-alkoxy, C₁-C₁₀-haloalkoxy, C₂-C₁₀-alkenyloxy, C₂-C₁₀-alkynyloxy, C₃-C₁₀-haloalkynyloxy, C₁-C₁₀-alkylthio, C₁-C₁₀-haloalkylthio, C₁-C₁₀-alkylsulfinyl, C₁-C₁₀-haloalkylsulfinyl, C₁-C₁₀-alkylsulfonyl, C₁-C₁₀-haloalkylsulfonyl, hydroxy, NR⁷R⁸, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, R⁷R⁸N—CO—, phenyl, benzyl and phenoxy, wherein phenyl, benzyl and phenoxy may be unsubstituted or substituted by 1, 2, 3, 4 or 5 substituents R^{b} as defined above;
R⁶ is selected from the group consisting of H, C₁-C₁₀-alkyl, which is optionally substituted by CN or NO₂, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₂-C₁₀-haloalkynyl, C₁-C₁₀-alkylsulfinyl, C₁-C₁₀-haloalkylsulfinyl, C₁-C₁₀-alkylsulfonyl, C₁-C₁₀-haloalkylsulfonyl, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl, C₁-C₁₀-haloalkylcarbonyl, hydroxy-C₁-C₁₀-alkyl, C₁-C₁₀-alkoxy-C₁-C₁₀-alkyl, halo-C₁-C₁₀-alkoxy-C₁-C₁₀-alkyl, C₁-C₁₀-alkoxycarbonyl-C₁-C₁₀-alkyl, halo-C₁-C₁₀-alkoxycarbonyl-C₁-C₁₀-alkyl R⁷R⁸N—CO— and benzyl wherein benzyl may be substituted by 1, 2, 3, 4 or 5 substituents R^{b} as defined above; and
R⁷, R⁸ are selected independently of one another from H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₂-C₁₀-alkynyl, C₃-C₁₀-haloalkynyl, phenyl or benzyl wherein phenyl and benzyl may be substituted by 1, 2, 3, 4 or 5 substituents which are selected, independently of one another, from halogen, cyano, nitro, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl, C₁-C₁₀-alkoxy, C₁-C₁₀-haloalkoxy, C₁-C₁₀-alkylthio, C₁-C₁₀-haloalkylthio, C₁-C₁₀-alkylsulfonyl, C₁-C₁₀-haloalkylsulfonyl, hydroxy, C₁-C₁₀-alkoxycarbonyl, C₁-C₁₀-haloalkoxycarbonyl, C₂-C₁₀-alkenyloxycarbonyl, C₂-C₁₀-haloalkenyloxycarbonyl, C₁-C₁₀-alkylcarbonyl and C₁-C₁₀-haloalkylcarbonyl.

85. The compound as claimed in claim 83, wherein Ar in formula I is phenyl, which is unsubstituted or substituted by 1, 2, 3 or 4 radicals R^{c}.

86. The compound as claimed in claim 83, wherein X in formula I is C═O.

87. The compound as claimed in claim 83, wherein A in formula I is a cyclic radical selected from phenyl, thienyl, furyl, pyrrolyl, pyrazolyl, imidazolyl, oxazolyl, isoxazolyl, thiazolyl, isothiazoloyl, pyridyl, pyrimidinyl, pyrazinyl, and pyridazinyl and where the cyclic radical may be substituted by 1, 2 or 3 substituents R^{a}.

88. The compound as claimed in claim 83, wherein A in formula I is a cyclic radical selected from phenyl, thienyl, and pyridyl, where the cyclic radical may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

89. The compound as claimed in claim 83, wherein A in formula I is 2-thienyl which may be substituted by 1, 2 or 3 substituents R^{a} which are selected, independently of one another, from halogen, C₁-C₄-alkyl, C₁-C₄-alkoxy, C₁-C₄-haloalkoxy and C₁-C₄-haloalkyl.

90. The compound as claimed in claim 83, wherein Q in formula I is selected from a single bond, —CH₂—, O—CH₂—, —S—CH₂—, —O—CH(CH₃)— and —S—CH(CH₃)—.

91. The compound as claimed in claim 83, wherein Q and A together are C₁-C₁₀-alkyl which may be substituted by 1 or 2 substituents selected from the group consisting of ═O, OH and C₁-C₂-alkoxy.

92. The compound as claimed in claim 83, wherein R² in formula I is selected from hydrogen, halogen and C₁-C₄-alkyl.

93. The compound as claimed in claim 83, wherein R³ in formula I is selected from hydrogen, halogen and C₁-C₄-alkyl.

94. The compound as claimed in claim 83, wherein R³ in formula I is hydrogen.

95. The compound as claimed in claim 83, wherein R⁴is hydrogen or a radical R^{c}.

96. The compound as claimed in claim 95, wherein. Ar is phenyl, which is unsubstituted or substituted by 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

97. The compound as claimed in claim 83, wherein R⁴ together with R² is —O—.

98. The compound as claimed in claim 97, wherein Ar in formula I is phenyl, which is unsubstituted or which may carry 1, 2 or 3 radicals R^{c} which are selected, independently of one another, from halogen, CN, C₁-C₄-alkyl, C₁-C₄-alkoxy, C₁-C₄-haloalkoxy and C₁-C₄-haloalkyl.

99. The compound as claimed in claim 83, wherein R⁵ is selected from the group consisting of H, C₁-C₁₀-alkyl, C₁-C₁₀-haloalkyl, C₃-C₁₀-cycloalkyl, C₃-C₁₀-halocycloalkyl, C₂-C₁₀-alkenyl, C₂-C₁₀-haloalkenyl.

100. The compound as claimed in claim 83, wherein R⁶ is hydrogen.

101. A composition for combating pests, selected from insects, arachnids and nematodes, which comprises a pesticidally effective amount of at least one compound of the general formula I as defined in claim 64 and at least one inert carrier and/or at least one surfactant.

102. A composition for combating pests, selected from insects, arachnids and nematodes, which comprises a pesticidally effective amount of at least one compound of the general formula I as defined in claim 83 and at least one inert carrier and/or at least one surfactant.
